(19) United States
(12) Patent Application Publication
(10) Pub. No.: US 2024/0374520 A1
(43) Pub. Date: Nov. 14, 2024

(54) PROTEOLIPID VESICLES FORMULATED WITH FUSION ASSOCIATED SMALL TRANSMEMBRANE PROTEINS

(71) Applicant: ENTOS PHARMACEUTICALS INC., Edmonton (CA)

(72) Inventors: John David Lewis, Encinitas, CA (US); Arun Raturi, Edmonton (CA)

(21) Appl. No.: 18/613,605

(22) Filed: Mar. 22, 2024

Related U.S. Application Data
(63) Continuation of application No. 18/029,823, filed on Mar. 31, 2023.

(30) Foreign Application Priority Data
Oct. 1, 2020 (CA) 3094859

(51) Int. Cl. A61K 9/127; A61K 31/7105; A61K 31/711; A61K 47/64; A61K 48/00

(52) U.S. Cl. CPC A61K 9/1271; A61K 31/7105; A61K 31/711; A61K 47/64; A61K 48/0033

(57) ABSTRACT

Disclosed is a proteolipid vesicle (PLV) for delivering a therapeutic cargo, such as nucleic acids, polypeptides and molecules, to a cell, the proteolipid vesicle having a lipid nanoparticle comprising one or more ionizable lipids and one or more of a fusion associated small transmembrane (FAST) family of proteins and chimerics thereof, where the molar ratio of ionizable lipid to therapeutic cargo is between 2.5:1 and 20:1. Incorporation of a chimeric FAST protein into a PLV platform enhances intracellular delivery and expression of mRNA and pDNA both in vitro and in vivo. These PLVs also display a favorable immune profile and are significantly less toxic than conventional LNPs.

### FIELD OF THE INVENTION

The invention generally relates to lipid nanoparticle delivery platforms. More specifically, the invention relates to a proteolipid vesicle and compositions comprising thereof, having both ionizable lipids and fusion-associated transmembrane proteins that is able to encapsulate and deliver a therapeutic cargo into cells.

### BACKGROUND

The potential for gene therapy to treat a myriad of diseases ranging from monogenic disease to cancer has resulted in over 2600 gene therapy clinical trials, culminating in only ten nucleic acid drugs receiving regulatory approval by the USA Food and Drug Administration (FDA) (1). The plasma membrane is a highly effective physical barrier to exogenous and charged macromolecules like negatively charged nucleic acids. Viral vectors have dominated gene therapy trials worldwide due to their high efficiency of gene expression and have demonstrated success clinically. The approval of alipogene tiparvovec (Glybera) to treat lipoprotein lipase deficiency in 2012 catalyzed an industry shift towards the use of adeno-associated virus (AAV) vectors (2, 3). Though safer than many traditional viral vectors, AAV use is limited by the existence of neutralizing antibodies with no prior AAV vector exposure (4-7). Host immunogenic responses against the AAV vector can also inhibit gene transfer following repeat dosing unless multiple AAV serotypes are employed (8, 9). Clinical trials have demonstrated the success of systemic AAV gene therapy for the treatment of diseases such as hemophilia ( ) and mucopolysaccharidoses (NCT03612869). A recent clinical trial exploring the use of AAV to deliver monoclonal IgG1 antibody against the HIV-1 gp120 protein demonstrates this, as an anti-AAV immune response resulted in low gene transfer and expression (10). To overcome these limitations, the focus in recent years has largely shifted to the development and optimization of non-viral delivery vectors.

Non-viral delivery vectors such as lipid nanoparticles (LNPs) are traditionally used for RNA-based gene therapy approaches (siRNA, miRNA, mRNA) and have cost, manufacturing, and immunogenicity advantages over viral vectors (11-17). The recent FDA approval of patisiran (Onpattro) has set the stage for more systemic non-viral nucleic acid therapies in the near-future (18). LNPs are formulated with cationic or ionizable lipids that neutralize the anionic charge of nucleic acids and facilitate the endosomal escape of encapsulated nucleic acids through charge-mediated lipid bilayer disruption (19-22). Onpattro utilizes the ionizable lipid DLin-MC3-DMA (MC3), which can be utilized for the delivery of mRNA; an approach extensively studied for developing vaccines (22, 23). MC3 becomes positively charged in the acidic endosomal compartment, facilitating endosomal escape. While ionizable lipids have substantially improved tolerability compared to cationic lipids, their mechanism of action potentiates apoptotic cell death, which translates to tolerability challenges after local delivery and dose-limiting liver toxicity following systemic delivery (23-25).

Though largely considered non-immunogenic, interactions between LNPs and the immune system can have detrimental systemic effects and lead to secretion of proinflammatory cytokines like tumor necrosis factor alpha (TNF-α), interferon-gamma (IFN-γ), and interleukin-6 (IL-6) (26, 27).

LNPs are nanostructures that are composed of a combination of different classes of lipids such as a cationic or ionizable lipid (CIL), structural lipids (phospholipid and sterol lipid) and PEG-conjugated lipid (PEG-lipid). These lipids self-assemble into LNPs under controlled microfluidic mixing with an aqueous phase containing the nucleic acids. The most critical component of an LNP, the CIL is responsible for electrostatically binding with the nucleic acids and encapsulating them. These CILs are also responsible for facilitating the endosomal escape of the nucleic acids. PEG-lipids prevent aggregation, degradation, and opsonization of the LNPs, while the structural lipids promote the stability and integrity of the nanoparticle.

As opposed to positively charged cationic lipids, the charge of ionizable lipids is dependent upon the pH of the surrounding environment. Ionizable lipids are composed of three sections: the amine head group, the linker group and the hydrophobic tails. Lipids with a small head group and tails composed of unsaturated hydrocarbons tend to adopt a conical structure, whereas lipids with a large head group and saturated tails tend to adopt a cylindrical structure.

Some lipids used in LNP formulations are immunogenic, this can be problematic for LNPs used in gene therapy, but advantageous for LNP vaccines and suggests that strategic use of different types of lipids in LNP formulations depending on clinical usage could greatly improve the safety and efficacy of the final product (28). Cationic lipids have been shown to activate Toll-like receptor 4, which in turn promotes a strong pro-inflammatory response with induction of Th1 type cytokines IL-2, IFN γ and TNF a (21). Compared to neutral or anionic LNPs, intravenously injected cationic LNPs have also been reported to induce an IFN-I response and elevated levels of interferon responsive gene transcripts in leukocytes. Cationic lipids have been added to protein-liposome vaccines to act as adjuvants and stimulate a bigger Th1 immune response (29), while avoiding overstimulation of a Th2 immune response (production of IL-5 and IL-13) implicated with vaccine immunopathology (30). The type of cationic liposome also greatly effects the immune activation by liposome-DNA complexes, for example non-CpG containing Lipofectamine2000 liposomes induced 5× more cytokine production than either DOTMA/DOPE or DOTMA/CHOL liposomes (31). Lipofectamine2000 liposomes containing non-CpG motif DNA also induced IFN-β and IL-6 production by macrophages from TLR9 deficient mice (31). Some anionic liposome-protein antigen mixes showed comparable adjuvant activity to that of cationic lipids in cancer vaccine development. The anionic lipid DOPA admixed with ovalbumin induced antigen-specific CD8(+) cytotoxic T lymphocyte responses and significantly delayed the growth of OVA-expressing B16-OVA tumors in mice (29).

The levels of cytokine production may also be LNP dosage dependent (or dependent on the molar ratio of cationic to neutral lipids in the LNP formulation) which be advantageous for low systemic dose vaccine activity, but dangerous with the higher systemic doses generally used in gene therapy.

Furthermore, LNPs can stimulate complement activation-related pseudoallergy (CARPA), a hypersensitivity reaction resulting in death in severe circumstances (32-34). The use of ionizable lipids has addressed some of the limitations surrounding LNP use, however, there are still safety concerns that has restricted their success clinically. Despite the unprecedented research investment, gene therapy is still considered experimental by the FDA with the primary limitation being the lack of a safe and effective vehicle platform for intracellular nucleic acid delivery and with a wider biodistribution other than the liver.

However, significant safety concerns have been raised over the use of viral vectors (3, 41). Preclinical data has demonstrated the potential for insertional mutagenesis from viral vectors conferring a cancer risk that is further perpetuated by their relative lack of target selectivity (42, 43). The most significant barrier for viral vectors is stimulation of an immune response, which promotes harmful side effects and blocks gene transfer from the virus. For example, the efficacy of adeno-associated virus (AAV) is limited by host humoral immune responses and viral delivery platforms are also costly to produce and have inherent cargo-size constraints.

Intracellular nucleic acid release is imperative for their function, however, using cationic and ionizable lipids can be counterproductive as they stimulate significant toxic and immunogenic responses, curtailing their use clinically.

Alternative nucleic acid delivery strategies that do not rely on toxic components need to be developed to ensure widespread success of gene, small molecule, nucleic acid, polypeptide delivery therapies and nucleic acid vaccines.

With the risk of systemic toxicity, many ionizable lipid formulations have been repurposed for intramuscular injection. Bahl et al. (44) developed a messenger RNA (mRNA) based influenza vaccine formulated with the ionizable lipid DLin-MC3-DMA (MC3). Safety concerns about LNPs and other non-viral vectors continue to hinder their success clinically (45).

### SUMMARY OF THE INVENTION

It is an object of the present invention to provide compositions and/or proteolipid vesicles for delivering a therapeutic cargo to a cell.

According to an aspect of the present invention there is provided a proteolipid vesicle for delivering a therapeutic cargo, such as nucleic acids, polypeptides and molecules, to a cell, the proteolipid vesicle having a lipid nanoparticle comprising one or more ionizable lipids and one or more of a fusion associated small transmembrane (FAST) family of proteins and chimerics thereof, wherein the molar ratio of ionizable lipid to nucleic acid is between 2.5:1 and 20:1.

FAST proteins are the only examples of membrane fusion proteins encoded by nonenveloped viruses, and at ˜100-200 residues in length are the smallest known viral fusogens. These non-glycosylated proteins are not components of the virion but are expressed inside virus-infected cells and trafficked to the plasma membrane where they mediate cell-cell membrane fusion, generating multinucleated syncytia to promote cell-cell virus transmission (37). FAST proteins function at physiological pH and do not require specific cell receptors, allowing them to fuse almost all cell types (38).

Structure-function relationships between different FAST proteins have indicated that overlapping structural motifs of FAST can be exchanged with other FAST proteins to generate a functional fusion protein (39, 40), referred to herein as a chimeric FAST protein. Chimeric FAST proteins display superior fusion activity. When a chimeric FAST protein is incorporated into a proteolipid nucleic acid delivery vehicle (PLV) it enables a minimal molar ratio of ionizable lipid to be used for the sole purpose of neutralizing the anionic charge of the nucleic acid, rather than facilitating endosomal escape. These FAST-PLVs display a favorable toxicity profile while maintaining efficient systemic gene expression by capitalizing on the elegant fusion inducing properties of the Orthoreovirus FAST protein.

Incorporation of a chimeric FAST protein into a PLV platform enhances intracellular delivery and expression of mRNA and pDNA both in vitro and in vivo. These PLVs also display a favorable immune profile and are significantly less toxic than conventional LNPs.

Disclosed is an approach to achieve systemic nucleic acid delivery by combining the fusion-inducing activities of FAST proteins with the improved safety and scalability of lipid-based non-viral delivery vectors. Given the small size of FAST-PLVs, as well as their efficacy, low immunogenicity, high tolerability, and the ability to reach extrahepatic tissues, FAST-PLVs should have substantial clinical utility, enabling the development of low-cost genetic medicines, therapeutics, and vaccines in the near future.

According to an aspect of the present invention, there is provided a proteolipid vesicle for delivering a therapeutic cargo to a cell comprising: a lipid nanoparticle comprising one or more ionizable lipids; and one or more of a fusion associated small transmembrane (FAST) family of proteins and chimerics thereof. The molar ratio of ionizable lipid to therapeutic cargo is between 2.5:1 and 20:1.

In one embodiment, the one or more ionizable lipids is Dlin-KC2-DMA (KC2), DODMA, DODAP, DOBAQ, DOTMA, 18:1 EPC, DOTAP, DDAB, 18:0 EPC, 18:0 DAP or 18:0 TAP. Preferably, the one or more ionizable lipids is DODAP and/or DODMA.

In another embodiment, the FAST protein is p10, p13, p14, p15, p16, p22, or chimerics thereof.

Preferably, the FAST protein is a p14p15 chimera, p10/p14 chimera or a p10/p15 chimera.

More preferably, the p14p15 chimera comprises the ectodomain and transmembrane of p14 and the endodomain of p15; the ectodomain of p14, the transmembrane domain and endodomain of p15; or the ectodomain and endodomain of p14 and the transmembrane of p15.

In a further embodiment, the proteolipid vesicle contains the therapeutic cargo. The therapeutic cargo being a nucleic acid, polypeptides or molecule, where the nucleic acid is pDNA, mRNA, siRNA, miRNA, self-amplifying mRNA, genetic adjuvants, promoters or molecular gene editing tools; the polypeptide is a peptide, epitope, or antigenic molecule; and, the molecule is a small drug molecule, biological molecule, structural macromolecule, or therapeutic molecule.

In a still further embodiment, the molar ratio of ionizable lipid to therapeutic cargo is 5:1, 7.5:1, 10:1 or 15:1.

In one embodiment, the lipid nanoparticle comprises DOTAP, DODAP, DOPE and DMG-PEG, preferably in a mole percentage of 24:42:30:4.

In a second embodiment, the lipid nanoparticle comprises DOTAP, DODMA, DOPE and DMG-PEG, preferably in a mole percentage of 24:42:30:4.

In a third embodiment, the lipid nanoparticle comprises DOTAP, DODAP, DODMA, DOPE and DMG-PEG, preferably in a mole percentage of 24:21:21:30:4.

In a fourth embodiment, the lipid nanoparticle comprises DOTAP, DODAP, DOPE and DMG-PEG, in a mole percentage of 6:60:30:4 or 3:63:30:4.

In a fifth embodiment, the lipid nanoparticle comprises DODAP, DOPE and DMG-PEG, preferably in a mole percentage of 66:30:4.

In a sixth embodiment, the lipid nanoparticle comprises DODAP, cholesterol, DOPE and DMG-PEG, preferably in a mole percentage of 49.5:24.75:23.75:2, 49.5:38.5:10:2 or 61.7:26.3:19:3.

In a further embodiment, the proteolipid vesicle further comprising bombesin attached to the C-terminal of the FAST protein or chimeric thereof.

According to another aspect of the present invention, there is provide a composition for delivering a therapeutic cargo to a cell comprising: the proteolipid vesicle as described above; and a therapeutic cargo encapsulated by the proteolipid vehicle.

In an embodiment, the therapeutic cargo is a nucleic acid, polypeptide or molecule, or a combination thereof. The therapeutic cargo being a nucleic acid, polypeptides or molecule, where the nucleic acid is pDNA, mRNA, siRNA, miRNA, self-amplifying mRNA, genetic adjuvants, promoters or molecular gene editing tools; the polypeptide is a peptide, epitope, or antigenic molecule; and, the molecule is a small drug molecule, biological molecule, structural macromolecule, or therapeutic molecule.

In one embodiment, the lipid nanoparticle comprises DOTAP:DODAP:DOPE:DMG-PEG in a mole percentage of 24:42:30:4 and the molar ratio of ionizable lipid to pDNA is between 5:1 and 10:1.

In a second embodiment, the lipid nanoparticle comprises DOTAP:DODAP:DOPE:DMG-PEG in a mole percentage of 24:42:30:4 and the molar ratio of ionizable lipid to mRNA is between 5:1 and 10:1.

In a third embodiment, the lipid nanoparticle comprises DOTAP:DODMA:DOPE:DMG-PEG in a mole percentage of 24:42:30:4 and the molar ratio of ionizable lipid to pDNA is between 4:1 to 7.5:1.

In a fourth embodiment, the lipid nanoparticle comprises DOTAP:DODMA:DOPE:DMG-PEG in a mole percentage of 24:42:30:4 and the molar ratio of ionizable lipid to mRNA is between 2.5:1 to 7.5:1.

In a fifth embodiment, the lipid nanoparticle comprises DOTAP:DODAP:DODMA:DOPE:DMG-PEG in a mole percentage of 24:21:21:30:4 and the molar ratio of ionizable lipid to pDNA is between 3:1 to 7.5:1.

In a sixth embodiment, the lipid nanoparticle comprises DOTAP:DODAP:DODMA:DOPE:DMG-PEG in a mole percentage of 24:21:21:30:4 and the molar ratio of ionizable lipid to mRNA is between 2.5:1 to 7.5:1.

In a seventh embodiment, the lipid nanoparticle comprises DOTAP:DODAP:DOPE:DMG-PEG in a mole percentage of 3:63:30:4 and the molar ratio of ionizable lipid to pDNA is between 7.5:1 to 15:1.

In an eighth embodiment, the lipid nanoparticle comprises DOTAP:DODAP:DOPE:DMG-PEG in a mole percentage of 3:63:30:4 and the molar ratio of ionizable lipid to pDNA is between 5:1 to 12:1.

In a ninth embodiment, the lipid nanoparticle comprises DOTAP:DODAP:DOPE:DMG-PEG in a mole percentage of 6:60:30:4 and the molar ratio of ionizable lipid to pDNA is between 5:1 to 15:1.

In a tenth embodiment, the lipid nanoparticle comprises DOTAP:DODAP:DOPE:DMG-PEG in a mole percentage of 6:60:30:4 and the molar ratio of ionizable lipid to mRNA is between 5:1 to 15:1.

In an eleventh embodiment, the lipid nanoparticle comprises DODAP:DOPE:DMG-PEG in a mole percentage of 66:30:4 and the molar ratio of ionizable lipid to pDNA is between 5:1 to 20:1.

In a twelfth embodiment, the lipid nanoparticle comprises DODAP:DOPE:DMG-PEG in a mole percentage of 66:30:4 and the molar ratio of ionizable lipid to mRNA is between 5:1 to 20:1.

In a thirteenth embodiment, the lipid nanoparticle comprises DODAP:cholesterol:DOPE:DMG-PEG in a mole percentage of 49.5:24.75:23.75:2 and the molar ratio of ionizable lipid to pDNA is between 5:1 to 15:1.

In a fourteenth embodiment, the lipid nanoparticle comprises DODAP:cholesterol:DOPE:DMG-PEG in a mole percentage of 49.5:24.75:23.75:2 and the molar ratio of ionizable lipid to mRNA is between 2.5:1 to 15:1.

In a fifteenth embodiment, the lipid nanoparticle comprises DODAP:cholesterol:DOPE:DMG-PEG in a mole percentage of 49.5:38.5:10:2 and the molar ratio of ionizable lipid to pDNA is between 5:1 to 15:1.

In a sixteenth embodiment, the lipid nanoparticle comprises DODAP:cholesterol:DOPE:DMG-PEG in a mole percentage of 49.5:38.5:10:2 and the molar ratio of ionizable lipid to mRNA is between 2.5:1 to 15:1.

In a seventeenth embodiment, the lipid nanoparticle comprises DODAP:cholesterol:DOPE:DMG-PEG in a mole percentage of 61.7:26.3:19:3 and the molar ratio of ionizable lipid to pDNA is between 5:1 to 15:1.

In an eighteenth embodiment, the lipid nanoparticle comprises DODAP:cholesterol:DOPE:DMG-PEG in a mole percentage of 61.7:26.3:19:3 and the molar ratio of ionizable lipid to mRNA is between 2.5:1 to 15:1.

In a further embodiment, the therapeutic cargo is c14orf132 siRNA.

According to an aspect of the present invention, there is provided use of the composition as described above to deliver a therapeutic cargo to a host cell.

In an embodiment, the therapeutic cargo is a nucleic acid, polypeptide or molecule. The therapeutic cargo being a nucleic acid, polypeptides or molecule, where the nucleic acid is pDNA, mRNA, siRNA, miRNA, self-amplifying mRNA, genetic adjuvants, promoters or molecular gene editing tools; the polypeptide is a peptide, epitope, or antigenic molecule; and, the molecule is a small drug molecule, biological molecule, structural macromolecule, or therapeutic molecule.

In another embodiment, the host cell is a cancer cell, immortalized cell, primary cell or muscle cell. Preferably, the host cell is an immortalized or primary cell.

According to a further aspect of the present invention, there is provided use of the composition of as described above containing c14orf132 siRNA for treatment of a metastatic cancer.

According to a still further aspect of the present invention, there is provided a method of delivering a therapeutic cargo to a host cell, comprising: administering the composition as described above to a cell.

In an embodiment, the therapeutic cargo is a nucleic acid, polypeptide or molecule. The therapeutic cargo being a nucleic acid, polypeptides or molecule, where the nucleic acid is pDNA, mRNA, siRNA, miRNA, self-amplifying mRNA, genetic adjuvants, promoters or molecular gene editing tools; the polypeptide is a peptide, epitope, or antigenic molecule; and, the molecule is a small drug molecule, biological molecule, structural macromolecule, or therapeutic molecule.

In another embodiment, the host cell is a cancer cell, immortalized cell, primary cell or muscle cell. Preferably, the host cell is an immortalized or primary cell.

### BRIEF DESCRIPTION OF THE DRAWINGS

The embodiments of the present disclosure will be described with reference to the following drawings wherein:

FIG. 1 shows the engineering of p14endo15 proteolipid vehicles (p14endo15-PLVs). (a) Arrangement of the ectodomain, transmembrane (TM) domain, and endodomain fusion modules of the parental p14 and p15 FAST proteins and various chimeric constructs. Color scheme depicts the wildtype source of each fusion module (p14, green; p15, blue). The locations of the N-terminal myristylation site and adjacent fusion peptide motif in the ectodomain, and the endodomain polybasic motif (++) and adjacent amphipathic helix are indicated. Chimeras were named with the FAST protein contributing two domains as the backbone followed by the inserted domain name abbreviation and FAST protein identity. Numbers indicate the number of residues in each protein. Syncytium formation of the various constructs was scored on a 4+ scale with ‘-’ indicating no syncytium formation. (b) Representative images of Giemsa stained QM5 cells transfected with pcDNA3 expressing either p14, p15, or p14endo15 captured at 13 hours post-transfection. Arrows indicate syncytial nuclei. (c) Quantification of syncytium formation expressed as a percentage of syncytial nuclei over total nuclei. Data are represented as mean±standard deviation. One-way ANOVA and Tukey's multiple comparisons test. ****P<0.0001;

FIG. 2 shows the in vitro validation of p14endo15-PLVs. (a) Transmission electron microscope images of optimized lipid formulation 41N with p14endo15 encapsulating pDNA. (b-c) Atomic force microscopy used to assess the (B) 2D and (C) 3D structure of optimized lipid formulation 41N with p14endo15 encapsulating pDNA. (d) Optimized lipid formulation 41N with (right) and without (left) FAST protein (p14endo15) was used to encapsulate pDNA-GFP and 0.9 nM was incubated with IMR-90, primary rat hepatocytes, primary rat astrocytes, HUVECs, and HEP3B cells for 96 hours before fluorescence images were taken, and flow cytometry was conducted. Mean fluorescence intensity (MFI) is presented for the GFP⁺ fraction for all other cell types except for rat astrocytes, where it is presented for the total cell population due to low transfection efficiency in the −FAST group. (e) Ability of FAST protein to reduce the amount of ionizable lipid and improve expression from pDNA-FLuc in vitro. ARPE-19 cells were incubated with pDNA-FLuc encapsulated within optimized lipid formulation 41N with and without FAST protein at different ionizable lipid:pDNA molar ratios for 96 hours before luminescence was determined. (f) Cell viability of HUVEC treated with varying amounts of pDNA encapsulated in Lipofectamine 2000, MC3-LNPs, and FAST-PLVs. (g) Expression of pDNA-FLuc in IMR-90 cells delivered by MC3-LNPs or FAST-PLVs. (h) In vitro expression of mRNA-FLuc encapsulated within optimized lipid formulation 41N at multiple ionizable lipid:mRNA molar ratios made with and without FAST protein 48 hours after addition to WI38 cells. Data are represented as the mean±standard deviation. (i) Optimized lipid formulation 41N with and without FAST protein was used to encapsulated mRNA-mCherry at the optimal 3:1 molar ratio and incubated with HUVEC for 48 hours before fluorescence images were taken, and flow cytometry was conducted. Median fluorescent intensity is presented for the total cell population due to low transfection efficiency in the FAST group. (j) Ability of FAST-PLVs to deliver two different mRNA molecules to the same cell. HEP3B cells were incubated with FAST-PLVs encapsulating mRNA-eGFP and mRNA-mCherry for 48 hours before fluorescence images were taken, and flow cytometry was conducted. (k) Ability of FAST-PLVs to delivery both pDNA-GFP and mRNA-mCherry to the same cell. HEP3B cells were incubated with FAST-PLVs encapsulating pDNA-GFP and mRNA-mCherry for 72 hours before fluorescence images were taken, and flow cytometry was conducted;

FIG. 3 shows the safety and efficacy validation of p14endo15-PLVs encapsulating pDNA-FLuc in mice. (a) Post-mortem liver images and hematoxylin and eosin staining of liver sections from mice injected with multiple doses of FAST-PLVs or MC3-LNPs encapsulating pDNA-FLuc (magnification=10×). (b)-(h) Blood samples were collected from mice 24 hours after injection with PBS, MC3-LNPs, or FAST-PLVs encapsulating pDNA-FLuc and Meso Scale Discovery V-PLEX was used to determine serum concentrations of pro-inflammatory cytokines: (b) TNF-α, (c) IL-6, (d), IFN-γ, (e) IL-1β, (f) CXCL1, (g) IL-10, (h) IL-5. Data are represented as mean±standard deviation, n=3 biologically independent mice per group, one-way ANOVA and Tukey's multiple comparisons test, *P<0.05, **P<0.01, ***P<0.001, ****P<0.0001, ns, not significant. (i) Whole body bioluminescent imaging of mice 24 hours after injection with pDNA-FLuc encapsulated within MC3-LNPs or FAST-PLVs. Mice injected intravenously with pDNA-FLuc encapsulated within MC3-LNPs received 0.5 mg/kg pDNA-FLuc, mice injected with FAST-PLVs encapsulating pDNA-FLuc received either 5 mg/kg or 20 mg/kg pDNA-FLuc. (j) Quantification of whole-body luminescence from mice in panel I (n=3 biologically independent mice per group). (k) Ex vivo organ bioluminescence from mice injected intravenously with pDNA-FLuc encapsulated within MC3-LNPs (0.5 mg/kg) or FAST-PLVs (5 mg/kg or 60 mg/kg). (l) Whole-body bioluminescence of mice injected intravenously with FAST-PLVs encapsulating 20 mg/kg pDNA-FLuc and monitored for one year. (m) Immunohistochemistry staining for FLuc 63 days after intravenous injection with FAST-PLVs encapsulating 20 mg/kg pDNA-FLuc (magnification=10×). TNF-α, tumor necrosis factor alpha; IFN-γ, interferon-gamma; CXCL1, chemokine (C-X-C motif) ligand 1;

FIG. 4 shows the safety and efficacy validation of p14endo15-PLVs encapsulating mRNA-FLuc in mice. (a) Incorporation of FAST protein into 41N lipid formulation improves the in vivo expression of mRNA-FLuc following intramuscular injection. Quantification of the bioluminescent signal from panel A. Data are represented as mean±standard deviation, n=3 biologically independent mice per group. Unpaired t-test, **P<0.01 (b) Whole-body bioluminescent of mice 4 hours after intravenous injection of FAST-PLVs encapsulating 2 mg/kg mRNA-FLuc. (c) Ex vivo organ bioluminescence of mice 4 hours after intravenous injection with FAST-PLVs encapsulating 2 mg/kg mRNA-FLuc. (d) Serum EPO concentrations following intravenous or intramuscular injection with FAST-PLVs encapsulating mRNA-EPO. n=3 biologically independent mice per group. (e) Mice repeatedly dosed intramuscularly with 0.3 mg/kg mRNA-FLuc encapsulated within FAST-PLVs, once a month for six months. (f) Quantification of whole-body bioluminescence from mice in panel E for 100 hours following injection. (g) Area under the curve calculated on time course shown in panels E and F. (h) Mice repeatedly dosed intravenously with 1.2 mg/kg mRNA-FLuc encapsulated in FAST-PLVs, once a month for six months. (i) Quantification of whole-body bioluminescence from mice in panel H for 100 hours following injection. (j) Area under the curve calculated on the time course shown in panels H and I. (k) Serum collected from mice dosed repeatedly intramuscularly or intravenously was assessed for anti-FAST antibody levels via ELISA. (l) Serum collected from repeatedly intramuscularly and intravenously dosed mice was assessed for anti-FLuc antibody levels via ELISA. (m) Serum from repeatedly dosed mice was incubated with FAST-PLVs encapsulating pDNA-GFP prior to addition to 3T3 cells. Flow cytometry was conducted 96 hours after addition and mean fluorescence intensity of the GFP⁺ population is presented. Pooled serum is a combination of equivalent volumes of serum from 10 separate animals with varying degrees of hemolysis to control for matrix interference;

FIG. 5 shows the safety validation of p14endo15-PLVs encapsulating pDNA-GFP in non-human primates. (a) Adult green monkeys (Chlorocebus sabaeus) were intravenously infused with FAST-PLVs encapsulating 1 mg/kg pDNA-GFP at a rate of 2 mL/min. Two days after infusion, the amount of pDNA was quantified in 30 tissues using a quantitative PCR approach with primers specific to the pDNA backbone. (b) Representative images of tissues stained with hematoxylin and eosin one day after intravenous infusion with FAST-PLVs encapsulating 1 or 6 mg/kg pDNA-GFP (magnification=10×). (c)-(d) Blood samples were collected from test subjects injected with FAST-PLVs encapsulating two doses of pDNA at indicated time points was used to determine the serum concentrations of the clinical chemistry parameters (c) ALT and (d) AST. Shaded area indicates normal parameter range. (e)-(g) Blood samples were collected from test subjects injected with FAST-PLVs encapsulating two doses of pDNA at indicated time points and Meso Scale Discovery V-PLEX was used to determine serum concentrations of pro-inflammatory cytokines: (e) IFN-γ, (f) IL-6, (g) IL-1β. (h)-(i), Blood samples were collected from test subjects injected with FAST-PLVs encapsulating two doses of pDNA at indicated time points and serum concentrations of the CARPA mediators, (h) C4d, and (i) SC5b-9, were determined via ELISA assay. ALT, alanine aminotransferase; AST, aspartate aminotransferase; IFN-γ, interferon-gamma;

FIG. 6 shows delivery of pDNA encoding follistatin using FAST-PLVs. (a) FAST-PLVs were used to encapsulate pDNA-CMV-FST (+) or pDNA-CMV-GFP (−) and added to C2C12 mouse myoblasts. Western blot was conducted to examine expression of FST as well as phosphorylation of Akt and mTOR. Numbers above phosphorylated bands represent fold increase relative to pDNA-CMV-GFP treated C2C12 lysates. (b) Media concentration of FST following FAST-PLV addition to C2C12 mouse myoblasts determined via ELISA. (c) Serum levels of FST following intravenous injection of C57Bl/6 mice with FAST-PLVs encapsulating 10 mg/kg pDNA-CMV-FST or pDNA-TTR-FST determined via ELISA (n=3 biologically independent mice per group). (d) Representative animals from each group 15 weeks following intravenous injection with PBS or FAST-PLVs encapsulating 10 mg/kg pDNA-CMV-FST or pDNA-TTR-FST. Note, hair loss in PBS mouse is likely unrelated to treatment as all cage mates presented with the same condition. (e) Body weight of mice 15 weeks following intravenous injection with PBS or FAST-PLVs encapsulating 10 mg/kg pDNA-CMV-FST or pDNA-TTR-FST. One-way ANOVA and Dunnett's multiple comparisons test, *P<0.05. (f) Body weight measurements from panel (E) were normalized to the initial body weight taken immediately prior to injection. One-way ANOVA and Dunnett's multiple comparisons test, *P<0.05. (g) Hindlimb grip strength of mice 15 weeks following intravenous injection with PBS or FAST-PLVs encapsulating 10 mg/kg pDNA-CMV-FST or pDNA-TTR-FST. One-way ANOVA and Dunnett's multiple comparisons test, **P<0.01. (h) Grip strength measurements from panel (G) were normalized to the initial grip strength reading taken immediately prior to injection. One-way ANOVA and Dunnett's multiple comparisons test, **P<0.01. (i) Hindlimb grip strength of mice injected intramuscularly into the left GAS with PBS or 5 mg/kg pDNA-CMV-FST 46 weeks after injection. Unpaired t-test, *P<0.05. (j) Gross dissection of mice 46 weeks after intramuscular injection with 5 mg/kg pDNA-CMV-FST into the left GAS (indicated with arrow) with representative WGA-488 stained GAS sections. (k) Ipsilateral and contralateral GAS weight 46 weeks after intramuscular injection with pDNA-CMV-FST or PBS. Unpaired t-test, **P<0.01. (1) Ipsilateral and contralateral GAS from pDNA-CMV-FST intramuscularly injected mice was stained with WGA-488 and cross-sectional muscle fiber area was determined using MyoVision Software. Unpaired t-test, ****P<0.0001. (m) Hindlimb grip strength of mice injected intravenously with 10 mg/kg pDNA-CMV-FST 46 weeks after injection. Unpaired t-test, **P<0.01. (n) Gross dissection of mice 46 weeks after intravenous injection with PBS or 10 mg/kg pDNA-CMV-FST with representative images of GAS WGA-488 staining. (o) GAS weight 46 weeks after intravenous injection with pDNA-CMV-FST or PBS. Unpaired t-test, *P<0.05. (p) Cross-sectional muscle fiber area of WGA-488 stained GAS from intravenously injected pDNA-CMV-FST or PBS mice was determined using MyoVision Software. Unpaired t-test, ****P<0.0001. FST, follistatin; GFP, green fluorescent protein; GAS, gastrocnemius muscle; WGA-488, wheat germ agglutinin Alexa Fluor 488; CMV, cytomegalovirus; TTR, transthyretin;

FIG. 7 shows synthesis and characterization of FAST-bombesin PLV. (a)-(h) Atomic Force Microscopy (AFM) of standard LNPs (a,c,e,g) and FAST PLVs (b,d,f,h). (i) Bombesin, a 14 amino acid ligand selectively binds gastrin-releasing peptide receptors (GRPR). (k)(a-d) FAST-bombesin protein retains fusion activity (syncytia assay);

FIG. 8 shows ligand-directed targeting of FAST-PLV. (a) Western blot of GRPR protein levels in PC3 and BPH cells. Beta tubulin loading standard. Cellular fluorescent microscopy showing enhanced delivery of FITC-Dextran to cells expressing GRPR. (b) Median fluorescent intensity of intracellular FITC labeled dextran delivered by standard LNPs, FAST-PLVs, and FAST-bombesin PLVs in BPH and PC3 cells. (c) Median fluorescent intensity of FAST-bombesin PLV delivered FITC labeled dextran in PC3 cells concomitantly treated with free bombesin peptide or GRPR siRNA;

FIG. 9 shows selective targeting of prostate cancer via GRSR receptors. Positron emission tomography (PET) imaging of PC-3 tumor uptake of 64Cu-labeled FAST-PLV formulations, (a, left panel) without fused bombesin and (right panel) with fused bombesin. (b), The standardized uptake value (SUV) showing the signal decay over 90 minutes;

FIG. 10 shows optimization of a cabazitaxel FAST-PLV. (a) POPC leads to PLVs with a smaller size and lower PDI than those formulated with DOPE. FAST protein did not significantly alter size, PDI, or zeta potential. (b) Incorporation of FAST protein into the DOPE based lipid formulation significantly enhanced the chemotoxicity;

FIG. 11 shows primary Tumor Growth and C14orf142 Expression. (a) Tumor growth (mm3), (b) body weight (g), (c) tumor weight (g), (d) tumor C14orf142 expression by RT-qPCR;

FIG. 12 shows 786-0 RCC organ metastasis for lung, brain, and liver. Human alu qPCR results showing the effect on the relevant metastatic burden after treatment with the negative control scramble carrying FAST-PLVs versus the C14orf142 siRNA carrying FAST-PLVs. n=10 animals. *p<0.05 vs Scramble, two tailed t-test;

FIG. 13 shows in vivo evaluation of siRNA (20875) delivery to the liver by FAST-PLV. siRNA 20875 targets a liver-expressed secreted protein called Gene X. The 20875 siRNA FAST-PLV formulation was intravenously injected at 3 mg/kg. The liver, lung, brain, and serum were collected from the mice 48 h post-injection. Significant reduction in circulating serum levels of Mouse Gene X after treatment with siRNA 20875 FAST-PLVs were determined. (a-b) Western blot of mouse liver, lungs and brain proteins, anti-Gene X antibodies were used to detect levels of expressed Gene X. (c) ELISA of mouse Gene X in serum. (d-e) Western blot of mouse serum proteins, anti-Gene X antibodies were used to detect levels of expressed Gene X;

FIG. 14 shows amino acid sequences for the (a) reptilian reovirus (python) p14 FAST protein, including the representative sequences for the ectodomain, transmembrane domain, amphipathic helix, and endodomain; and (b) the baboon reovirus p15 FAST protein including the representative sequences for the ectodomain, transmembrane domain, amphipathic helix (AH), and endodomain;

FIG. 15 shows schematics of authentic FAST proteins and endo-, transmembrane and ectodomain chimeric constructs with their relative fusion activities, (myr) myristic acid, (HP) hydrophobic patch, (ecto) ectodomain, (TM) transmembrane domain, (endo) endodomain, (••) palmitoylated cysteine residues, (+++) polybasic region. Fusion activities were qualitatively scored by observing the extent of syncytia formation in Giemsa-stained monolayers;

FIG. 16 shows the optimization of a lipid formulation for p14endo15-PLVs. (a) Tolerability comparison of 3 ionizable (DODAP, DLin-MC3-DMA, DODMA) and 2 cationic (DOTMA, DOTAP) lipids determined via Alamar Blue, 72 hours after their addition to WI-38 cells. (b) Effect of different ionizable lipid:pDNA molar ratios on the in vitro expression of pDNA-FLuc in ARPE-19 cells 96 hours after addition. (c)-(e) Comparison of the toxicity and efficacy of four lipid formulations (41N, 37N, 33T, 28M), formulated with FAST protein delivering 1.5 nM pDNA-FLuc to VERO cells. (c) LDH assay used to determine cytotoxicity 24 hours after pDNA-FLuc addition. (d) Luminescence determined 72 hours after pDNA-FLuc addition. (e) Expression as a function of cytotoxicity. Data are represented as the mean±standard deviation. One-way ANOVA, Tukey's multiple comparisons, ****P<0.0001. (f)-(l) Blood samples were collected from mice 24 hours after intravenous injection with each PLV lipid formulation encapsulating 8 mg/kg pDNA-FLuc and Meso Scale Discovery V-PLEX was used to determine serum concentrations of pro-inflammatory cytokines: (f) TNF-α, (g) IL-6, (h) IFN-γ, (i) IL-1β, (j) CXCL1, (k) IL-10, (l) IL-5. One-way ANOVA and Tukey's multiple comparisons test, *P<0.05, **P<0.01, ***P<0.001, ****P<0.0001. (m) Whole-body bioluminescence of mice injected intramuscularly FAST-PLVs encapsulating 1.25 mg/kg pDNA-FLuc, manufactured with lipid formulation 41N or lipid formulation 37N. (n) Quantification of the bioluminescent signal in panel M (n=2 biologically independent mice per group);

FIG. 17 shows the immunogenicity of p14endo15-PLVs following repeat intramuscular administration. Whole-body luminescence of mice injected intramuscularly with 10 μg mRNA-FLuc encapsulated within p14endo15-PLVs once a month for five months;

FIG. 18 shows the immunogenicity of p14endo15-PLVs following repeat intravenous administration. Whole-body luminescence of mice injected intravenously with 40 μg mRNA-FLuc encapsulated within p14endo15-PLVs once a month for five months;

FIG. 19 shows Formulation 37 is slightly more tolerable than 28 in vitro at delivering pDNA, and Formulation 41 most tolerated in human umbilical vein endothelial cells, generating highest mean fluorescent intensity and comparable transfection efficiency to MC3 when delivering pDNA-GFP;

FIG. 20 shows a separate experiment in HUVEC, formulation 41 demonstrated superior pDNA-GFP transfection when compared to a combination formulation composed of a 1:1 mixture of 37 and 33;

FIG. 21 shows SW80 cells transfected with Formulation 28 encapsulating mRNA-mCherry with different amounts of PEG. 4% PEG gave favourable sizing of ˜115 nm, with an encapsulation efficiency of 86.39%;

FIG. 22 shows In RPE cells, Formulation 28 demonstrated superior transfection of pDNA-FLuc. However, Formulation 41 consistently demonstrated an enhancement caused by p14e15. All formulations demonstrate some enhancement caused by p14e15 at lower charge ratios, indicating that fusion enhancing proteins may be effective at decreasing ionizable lipid amounts. Based on tolerability and p14e15 enhancement, Formulation 41 was examined extensively in vitro;

FIG. 23 shows IMR-90 cells were irradiated with 10Gy and left for 1 week, following which they were transfected with pDNA-Luc (top) or pDNA-GFP (bottom) encapsulated within Formulation 41. (top) 72 hours after transfection, luminescence was determined in irradiated and normal IMR-90 cells, demonstrating high expression in irradiated cells. (bottom) 72 hours after transfection, irradiated cells were stained with SA-3-Gal to identify senescent cells and imaging cytometry was conducted to determine GFP expression in SA-β-Gal+ and SA-β-Gal-cells. Note: other formulations lead to high degrees of toxicity in irradiated cells;

FIG. 24 shows the ability of FAST-PLVs (made with formulation 41N and p14endo15) to deliver pDNA encoding firefly luciferase by multiple administration routes—intramuscular and intravenous Injection, as well as oral administration;

FIG. 25 shows the durability of formulations of the present invention after subcutaneous administration. A single dose of formulations of the present invention encapsulating pDNA encoding CAG-luciferase. At day 55, no expression is observed outside of the injection site by ex vivo luminescence imaging; and

FIG. 26 shows the durability of PLV 41 after systemic administration. A single dose of formulations of the present invention encapsulating pDNA encoding CMV-luciferase in backbones with bacterial sequences (pcDNA3) or without (Nanoplasmid).

### DETAILED DESCRIPTION

The following description is of preferred embodiments by way of example only and without limitation to the combination of features necessary for carrying the invention into effect.

A proteolipid vehicle (PLV)-based therapeutic cargo delivery vehicle formulated with fusion associated small transmembrane (FAST) protein or a chimeric thereof that allows for the utilization of ionizable lipids at a minimal molar ratio for the sole purpose of neutralizing the anionic charge of therapeutic cargo, rather than using the ionizable lipids for facilitating endosomal escape. The incorporation of the described FAST protein or a chimeric thereof into the described PLV platform enhances intracellular delivery and expression of mRNA and pDNA both in vitro and in vivo. The PLVs of the present invention also display a favorable immune profile and are significantly less toxic than conventional LNPs.

The proteolipid vesicles of the present invention that are capable of delivering a therapeutic cargo to a cell comprise a lipid nanoparticle comprising one or more ionizable lipids; and one or more of a fusion associated small transmembrane (FAST) family of proteins and chimerics thereof. The molar ratio of ionizable lipid to therapeutic cargo is between 2.5:1 and 20:1.

Ionizable lipids are known in the art, and include, but are not limited to: Dlin-KC2-DMA (KC2), DODMA, DODAP, DOBAQ, DOTMA, 18:1 EPC, DOTAP, DDAB, 18:0 EPC, 18:0 DAP or 18:0 TAP. The proteolipid vesicles of the present invention preferably include DODAP and/or DODMA.

The FAST proteins of the present invention include native FAST proteins found in the family Reoviridae, including those from the genera Aquareovirus and Orthoreovirus. Aquareoviruses (AqRV) are primary responsible for infecting fish, whereas Orthoreoviruses (ORV) infect a number of vertebrate hosts including baboons (BRV, Baboon orthoreovirus), humans (MRV, Mammalian orthoreovirus), bats (NBV, Nelson Bay orthoreovirus; BrRV, Broome orthoreovirus), reptiles (RRV, Reptilian orthoreovirus) and domesticated land- and waterfowl (ARV, Avian orthoreovirus). In particular, the FAST proteins ARV p10, BrRv p13, RRV p14, BRV p15, AqV p16 and AqV p22 can be used in the PLV of the present invention.

FAST proteins are the only examples of membrane fusion proteins encoded by nonenveloped viruses, and at ˜100-200 residues in length are the smallest known viral fusogens. These non-glycosylated proteins are not components of the virion but are expressed inside virus-infected cells and trafficked to the plasma membrane where they mediate cell-cell membrane fusion, generating multinucleated syncytia to promote cell-cell virus transmission (37). FAST proteins function at physiological pH and do not require specific cell receptors, allowing them to fuse almost all cell types (38). FAST proteins share three common domains. A single transmembrane domain serves as a reverse signal-anchor sequence to direct a bitropic Nₒᵤₜ/Cᵢₙ type I topology in the membrane. This topology localizes a very small N-terminal ectodomain (20-40 residues) external to the plasma membrane and positions considerably longer (˜40-140 residues) C-terminal endodomains in the cytoplasm.

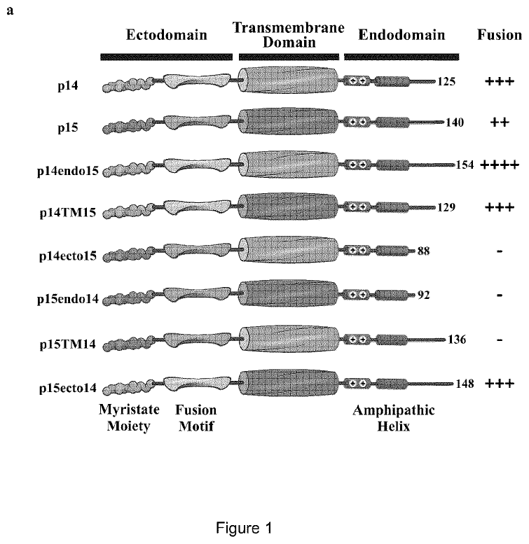

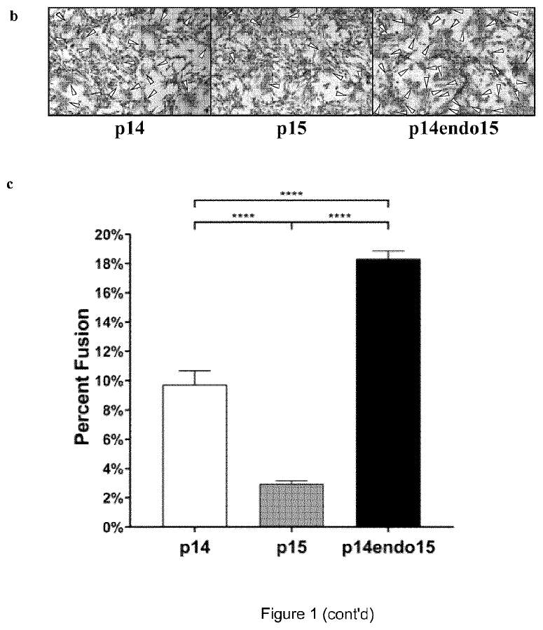

Chimeric FAST proteins can be synthesized that combine the domains from different FAST proteins, such the p10, p14 and p15 peptides, to form a functional polypeptide. For example, as shown in FIG. 1, a number of different chimeric FAST proteins have been synthesized. Of the different combinations, a chimeric comprising the ectodomain from the p14 FAST protein, or a functional portion thereof, the transmembrane domain from the p14 FAST protein, and the endodomain from the p15 FAST protein, or a functional portion thereof, referred to herein as “p14endo15”; a chimeric comprising the ectodomain from the p14 FAST protein, or a functional portion thereof, the transmembrane domain from the p15 FAST protein, and the endodomain from the p14 FAST protein, or a functional portion thereof, referred to herein as “p14TM15”; and a chimeric comprising the ectodomain from the p14 FAST protein, or a functional portion thereof, the transmembrane domain from the p15 FAST protein, and the endodomain from the p15 FAST protein, or a functional portion thereof, referred to herein as “p15ecto14” are particularly useful in the present invention.

Incorporation of a chimeric FAST protein into a PLV platform enhances intracellular delivery of the therapeutic cargo and expression of mRNA and pDNA both in vitro and in vivo. These PLVs also display a favorable immune profile and are significantly less toxic than conventional LNPs.

Although the PLV-FAST platform is particularly useful for the delivery of nucleic acid based therapeutic cargos, such as pDNA, mRNA, siRNA, miRNA, self-amplifying mRNA (SAM), genetic adjuvants, promoters, and molecular gene editing tools, the platform does also allow for the safe and effective delivery of polypeptides, such as peptides, epitopes, antigens, and molecules, such as small drug molecules, biological molecules, structural macromolecules, therapeutic macromolecules.

Genetic adjuvants are known in the art and include cargos in any of the above forms that can modulate immune responses when given along with a vaccine. Similarly, molecular editing tools are known in the art and include those tools that can be utilized to edit host genomes (ie: CRISPR/Cas9 technology). Biological molecules are similarly known in the art and include any substance produced/made by cells or living organisms. As would be known to a person skilled in the art, structural macromolecules are those macromolecules (lipids, proteins, carbohydrates and/or nucleic acids) that aid in structural integrity of the cell or organism. Similarly, therapeutic macromolecules are known in the art to include those molecules that can be used as therapeutics for disease and disorders.

Utilizing the membrane fusion inducing activity of FAST proteins, a highly tolerable therapeutic cargo delivery platform capable of systemic pDNA and mRNA delivery, for example, was developed. To achieve this, a library of chimeric FAST proteins was synthesized and screened. Proteo-lipid vehicles formulated with FAST proteins represent an effective and redosable therapeutic cargo delivery platform that enables broad biodistribution with high tolerability compared to conventional non-viral approaches. The discovery of a novel and highly active chimeric FAST fusogen, p14endo15, has enabled the re-imagining of the conventional lipid nanoparticle formulation to remove cholesterol (if desirable), utilize alternative ionizable lipids, and to select an optimal ratio of ionizable, helper, and PEGylated lipids to achieve these characteristics (FIG. 1). Without wishing to be bound by theory, the superior fusion activity of p14endo15 is likely mediated by the efficient p14 ectodomain fusion peptide and myristate moiety facilitating lipid mixing with the target cell membrane, followed by the p15 endodomain fusion-inducing lipid packing sensor (FLiPs) motif partitioning into the PLV membrane to promote pore formation and liposome-cell fusion activity (90, 91). Incorporation of p14endo15 into the PLV platform results in enhanced therapeutic cargo expression in vitro and in vivo.

Systemic in vivo administration of pDNA FAST-PLVs resulted in durable, dose-dependent expression of target proteins in a wide array of organs with no detectable tissue or immune toxicity, even at doses orders of magnitude higher than the maximum tolerated dose of conventional (clinically approved) LNP formulations. Conventional LNPs accumulate preferentially in the liver when administered systemically, mediated by ApoE binding to the LNP surface. This behavior has driven the clinical development of liver targeting siRNA-based LNP drugs (18, 92, 93), while limiting their broader application. Extrahepatic nucleic acid delivery is particularly important for indications such as cancer, which requires broad biodistribution to achieve sufficient uptake (3). Like conventional LNPs, most AAV serotypes tend to preferentially target the liver, except in the case of AAV9 that can target neurons, albeit with a lower efficiency of gene transfer (63, 94). This creates problems for other conditions, as gene transfer may require local AAV delivery that is not possible for all conditions (95). Additionally, cells with a high turnover rate will quickly dilute the transgene and due to immunogenic responses, the vector cannot be utilized again (96-99). This creates problems for development of genetic medicines targeting disseminated cancer. Based on the NHP biodistribution data that demonstrates quite extensive extrahepatic delivery of pDNA, FAST-PLVs should have substantial clinical utility in the treatment of advanced cancer.

The ability of FAST-PLVs administered locally or systemically to deliver pDNA encoding FST-344, and for gene delivery to effect quantifiable changes in muscle tissue similar to previous reports with AAV, demonstrates the potential clinical utility of this non-viral platform (103-106). Again, the low immunogenicity of FAST-PLVs is beneficial for this type of gene therapy. Where AAV essentially requires lifelong gene expression with a single dose, FAST-PLV administration can be adjusted to fit each patient need. This also enables treatment to be stopped and started as needed. The data discussed below also indicates that therapeutic gene expression following systemic FAST-PLV administration can be targeted to specific tissue types by altering the pDNA promoter, which can be utilized in the future to prevent off-target effects.

The successful in vivo delivery of pDNA, as the therapeutic cargo, using FAST-PLVs described herein represents a promising step forward for development of non-viral gene therapy approaches as DNA delivery has typically been restricted to viral platforms (3, 41). Additionally, FAST-PLVs were able to deliver mRNA to a wide array of extrahepatic organs. Typically, non-viral delivery vectors, such as LNPs, are only suitable for RNA-based gene therapy approaches due to challenges encapsulating large molecules like DNA (41). Because of its large size, pDNA requires higher molar ratios of cationic components to neutralize its anionic charge and facilitate delivery relative to particles encapsulating mRNA and siRNA. Increasing the amount of cationic components results in a corresponding increase in toxicity, preventing successful systemic delivery. FAST-PLVs represent one of the first non-viral nucleic acid delivery vehicles that can encapsulate pDNA and mRNA and is able to deliver both nucleic acids to the same cell.

Initial gene therapy trials using adenovirus vectors reported serious and life-threatening adverse events, instigating the shift to AAV vectors (2, 107, 108). While promising results have been obtained with AAV vectors, their small cargo capacity and anti-AAV immune responses limits their use (6, 108-110). The large cargo capacity of FAST-PLVs indicate a potential for development of CRISPR/Cas9 gene editing technologies (110, 111). Successful gene editing requires delivery of both Cas9 protein and a guide RNA (gRNA) to the target cell. Cas9 transgenes are approximately 4.2 kb, which puts them at the upper end of AAV packing capacity (112). To overcome this limitation, multiple AAV vectors are often required to deliver the Cas9 and gRNA separately (113), or a combination of LNPs and AAVs can be utilized (114). Additionally, modifications can be made to the Cas9 structure that enable the gRNA and Cas9 sequences to be included in the same AAV genome (109). On the other hand, utilizing FAST-PLVs would enable both Cas9 and gRNA sequences to be included on a single pDNA. Alternatively, Cas9 and gRNA could be co-encapsulated into the same PLV, which would enable the use of either pDNA or mRNA, or a combination of both.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein set forth, and as follows in the scope of the appended claims.

### Example 1: Engineering of a Novel FAST Protein Hybrid with Enhanced Fusion Activity

The FAST protein family comprises six structurally similar members named according to their molecular mass in Daltons (i.e., p10, p13, p14, p15, p16, and p22) and share no conserved sequence identity, including p10 (Avian orthoreovirus), p14 (Reptilian orthoreovirus), and p15 (Baboon orthoreovirus) (36). All six known FAST proteins show a bipartite membrane topology with the single transmembrane (TM) domain connecting a minimal N-terminal ectodomain of ˜19-40 residues to a longer C-terminal cytoplasmic endodomain (46). Structural motifs contained within these domains and the rate and extent of syncytium formation vary considerably between family members (47). FAST protein ectodomains typically have a myristate moiety on the penultimate N-terminal glycine and all contain diverse membrane-destabilizing fusion peptide motifs in their ectodomain (e.g., p14 proline-hinged loop, p15 type II polyproline helix) (48, 49). FAST protein endodomains all contain a juxtamembrane polybasic motif involved in protein trafficking (50), and a membrane-proximal membrane curvature sensor to drive pore formation (51). Along with specific TM domain features (40, 52), these motifs function in conjunction to remodel membranes and promote membrane fusion.

FAST proteins are modular because different elements or domains can perform redundant functions, for example the hydrophobic patch from all FAST proteins, the p15 proline-rich region, and the palmitoylated cysteine residues in the p10 endodomain are diverse domains that all destabilize membranes. As well, some elements, like the hydrophobic patch, retain the same fusion induction functionality while being located on different domains; for p14 it is on the ectodomain, while in p15 the hydrophobic patch is located on the endodomain (36, 46, 51).

To engineer a novel FAST protein hybrid with enhanced fusion activity, the modular nature of FAST proteins was used to determine whether a specific combination of motifs could be assembled into a recombinant FAST protein with enhanced fusion activity, starting with the high activity p14 and p15 FAST proteins (47). A chimeric p14 and p15 FAST protein library was generated where the ectodomain, TMD, or endodomain of p14 were substituted with the corresponding domain of p15, and the fusion activity of each construct was ranked using a syncytia formation assay (FIG. 1a). Chimeras with p15 TMD and p15 endodomain, either separately or together (p14TM15, p15ecto14), retained fusion activity. Meanwhile, no chimera with the p15 ectodomain out of context from the p15 backbone retained fusion activity (p14ecto15, p15endo14, p15TM14), which suggest that p15 ectodomain requires the trans located hydrophobic patch motif for fusion activity. Three of six chimeric proteins were fusion-dead while two maintained fusion activity comparable with the parental proteins. One candidate, p14endo15, had significantly higher activity than either p14 or p15 parent (FIG. 1b, c). This chimera comprises the p14 ectodomain, which contains a myristoylated proline-hinged loop fusion peptide motif with robust membrane-destabilizing activity, connected via the p14 TM domain to the p15 endodomain, which contains an efficient amphipathic helix-kink-helix membrane curvature sensor. Based on these results, p14endo15 was selected for the optimization of novel PLV formulations for delivery of a therapeutic cargo.

### Example 2: Optimization of Lipid and FAST Protein Formulations for Nucleic Acid Delivery

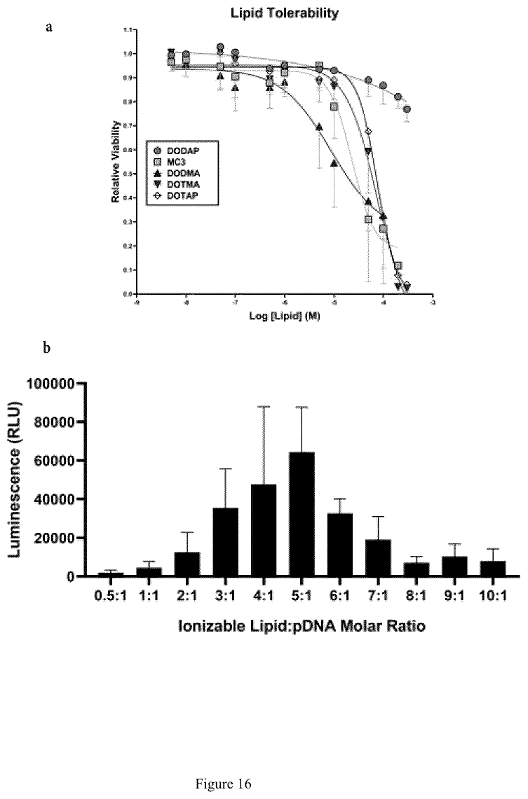

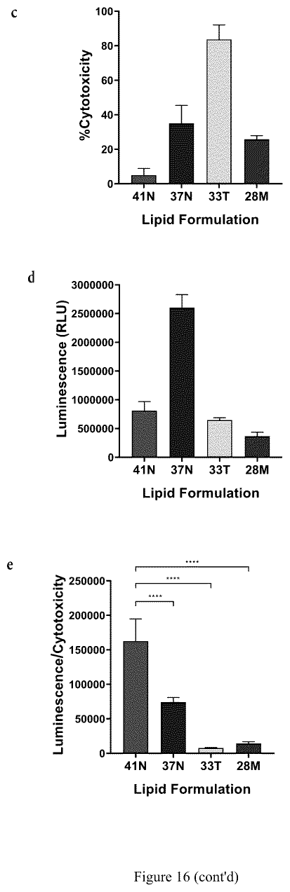

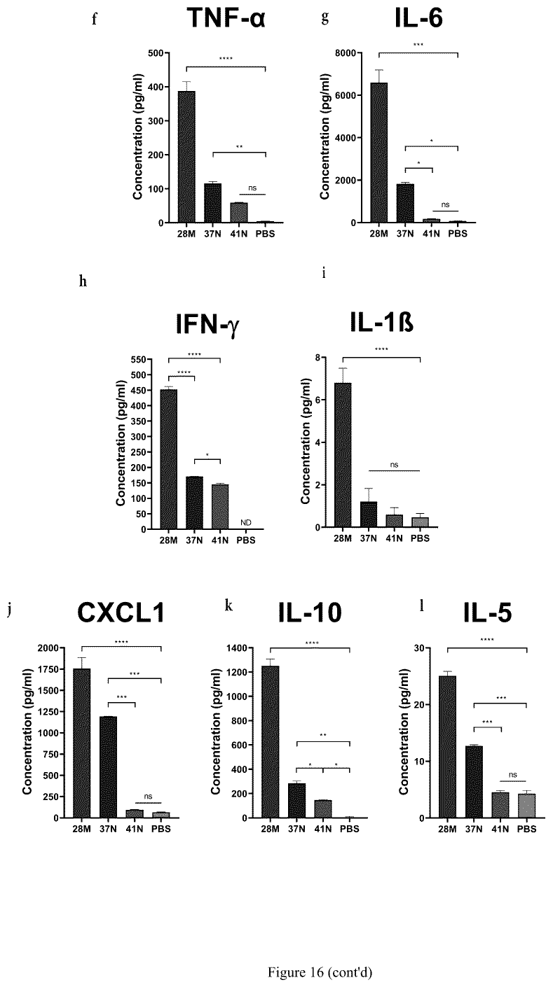

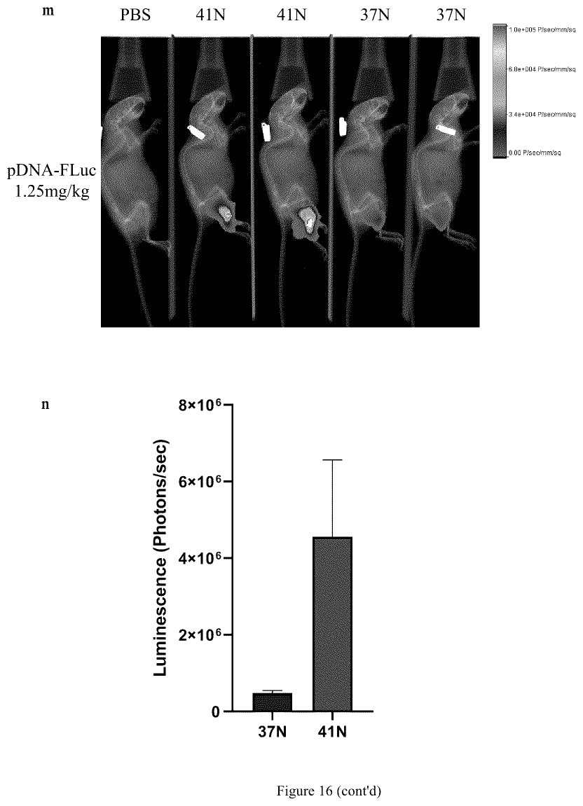

Conventional LNPs rely on endosomal escape for intracellular delivery, which results in limited nucleic acid release into the cytosol and is one of the major limitations of LNPs formulated for gene therapy (13, 20, 41). To deliver whether incorporating p14endo15 into a lipid formulation would significantly improve the delivery of nucleic acids into the cytosol by promoting PLV-cell membrane fusion, while changing the formulation requirements to promote improved tolerability compared to conventional LNPs due to the need for lipids to only promote nucleic acid encapsulation through charge neutralization, a panel of cationic and ionizable lipids was evaluated for their toxicity on human fibroblasts (WI-38), including cationic lipids 1,2-di-O-octadecenyl-3-trimethylammonium propane (DOTMA) and 1,2-dioleoyl-3-trimethylammonium-propane (DOTAP), as well as the ionizable lipids 1,2-dioleoyl-3-dimethylammonium-propane (DODAP), DLin-MC3-DMA (MC3), and 1,2-dioleyloxy-3-dimethylaminopropane (DODMA). Of these, DODAP had the most favorable tolerability, with DOTAP showing slightly higher toxicity (FIG. 16a). These lipids were utilized in a PLV formulation to promote nucleic acid encapsulation through charge neutralization. The optimal molar ratio of DODAP to pDNA was assessed by measuring the delivery and subsequent expression of DNA-encoded firefly luciferase (pDNA-FLuc) in retinal pigmented epithelial (ARPE-19) cells. A ratio of 5:1 ionizable lipid DODAP to pDNA resulted in maximal expression (FIG. 16b).

The lipid FAST protein formulation was optimized to determine the lowest molar ratio of cationic lipid that allows for efficient encapsulation of the anionic nucleic acid cargo coupled with the highest tolerability in vitro. A pDNA payload was then used to create a panel of more than 40 lipid formulations combining cationic lipid (DOTAP), ionizable lipid (DODAP and/or DODMA), cholesterol, helper lipid (2-dioleoyl-sn-glycero-3-phosphoethanolamine; DOPE), and PEGylated lipid (1,2-dimyristoyl-sn-glycero-3-methoxypolyethylene glycol-2000; DMG-PEG) at different ratios in an effort to balance intracellular delivery and activity with tolerability. Initially the most promising of these based on encapsulation and sizing (designated 28M, 33T, 37N and 41N) were produced using a microfluidic platform at the following lipid molar ratios (cationic/ionizable/helper/PEGylated): 28M (24:42:30:4), 33T (24:21:21:30:4), 37N (6:60:30:4) and 41N (0:66:30:4) (53-55). To assess the contribution of FAST protein to their delivery efficiency, pDNA-FLuc was encapsulated in each formulation with or without p14endo15 FAST protein and the resulting size, polydispersion index (PDI), and zeta potential was determined (Table 1).

TABLE 1
Physical characteristics of lipid nanoparticle formulations
with and without p14endo15 chimeric FAST protein.

| | Mean Size | Polydispersity | Zeta |
|---|---|---|---|
| Formulation | (nm) | Index | Potential |
| 28m | 62.2 ± 6.9 | 0.1525 ± 0.0058 | +0.560 ± 0.920 |
| 28m + p14endo15 | 52.7 ± 8.3 | 0.1865 ± 0.0064 | +1.738 ± 0.167 |
| 33T | 51.9 ± 3.0 | 0.2211 ± 0.0389 | +0.429 ± 0.166 |
| 33T + p14endo15 | 59.3 ± 5.0 | 0.1606 ± 0.0079 | +0.645 ± 0.746 |
| 37N | 75.8 ± 16.6 | 0.1730 ± 0.0044 | +0.886 ± 0.588 |
| 37N + p14endo15 | 52.4 ± 6.4 | 0.1740 ± 0.0159 | +6.442 ± 2.905 |
| 41N | 47.3 ± 6.2 | 0.2000 ± 0.0183 | −10.550 ± 0.573 |
| 41N + p14endo15 | 61.7 ± 11.7 | 0.1570 ± 0.0072 | −7.174 ± 1.631 |

All PLV formulations were found to be <80 nm in diameter with a PDI<0.3, indicating monodispersity. In subsequent experiments, further combinations of the various lipids were shown to be effective in vitro and in vivo, including formulations 38 (3:63:30:4), 410 (49.5:24.75:23.75:2), 410-1 (49.5:38.5:10:2), 41Cmp (61.7:26.3:19:3) (Table 2). All additional PLV formulations were found to be <80 nm in diameter with a PDI<0.3, indicating monodispersity.

TABLE 2
Composition of PLV Formulations

| | | | | Ionizable | Ionizable |
|---|---|---|---|---|---|
| | | | | Lipid:pDN | Lipid:mR |
| | | | | A Molar | NA Molar |
| Formulation | Composition | Mole % | Cargo | Ratio | Ratio |
| 28 | DOTAP: | 24:42:30:4 | pDNA, mRNA, siRNA, miRNA, | 5:1 to | 5:1 to |
| | DODAP: | | self-amplifying mRNA (SAM), | 10:1 | 10:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 29 | DOTAP: | 24:42:30:4 | pDNA, mRNA, siRNA, miRNA, | 4:1 to | 2.5:1 to |
| | DODMA: | | self-amplifying mRNA (SAM), | 7.5:1 | 7:5:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 33 | DOTAP: | 24:21:21:30:4 | pDNA, mRNA, siRNA, miRNA, | 3:1 to | 2.5:1 to |
| | DODAP: | | self-amplifying mRNA (SAM), | 7.5:1 | 7:5:1 |
| | DODMA: | | genetic adjuvants, promoters, | | |
| | DOPE: | | molecular gene editing tools, | | |
| | DMG-PEG | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 37 | DOTAP: | 6:60:30:4 | pDNA, mRNA, siRNA, miRNA, | 5:1 to | 5:1 to |
| | DODAP: | | self-amplifying mRNA (SAM), | 15:1 | 15:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 38 | DOTAP: | 3:63:30:4 | pDNA, mRNA, siRNA, miRNA, | 7.5:1- | 5:1- |
| | DODAP: | | self-amplifying mRNA (SAM), | 15:1 | 12:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 41 | DODAP: | 66:30:04 | pDNA, mRNA, siRNA, miRNA, | 5:1 to | 5:1 to |
| | DOPE: | | self-amplifying mRNA (SAM), | 20:1 | 20:1 |
| | DMG-PEG | | genetic adjuvants, promoters, | | |
| | | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 41C | DODAP: | 49.5:24.75:23.75:2 | pDNA, mRNA, siRNA, miRNA, | 5:1- | 2.5:1- |
| | Cholesterol: | | self-amplifying mRNA (SAM), | 15:1 | 15:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 41C-1 | DODAP: | 49.5:38.5:10:2 | pDNA, mRNA, siRNA, miRNA, | 5:1- | 2.5:1- |
| | Cholsterol: | | self-amplifying mRNA (SAM), | 15:1 | 15:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |
| 41Cmp | DODAP: | 61.7:26.3:19:3 | pDNA, mRNA, siRNA, miRNA, | 5:1- | 2.5:1- |
| | Cholesterol: | | self-amplifying mRNA (SAM), | 15:1 | 15:1 |
| | DOPE: | | genetic adjuvants, promoters, | | |
| | DMG-PEG | | molecular gene editing tools, | | |
| | | | peptides, polypeptides, epitopes, | | |
| | | | antigenic molecules, small drug | | |
| | | | molecules, biological molecules, | | |
| | | | structural macromolecules, | | |
| | | | therapeutic macromolecules | | |

The potency and cytotoxicity of these formulations were assessed in kidney epithelial (Vero) cells. Overall, formulation 41N showed the most favorable tolerability (FIG. 16c) and was slightly less potent than formulation 37N (FIG. 16d). While formulation 37N showed roughly 3-times higher potency, it was >6-times more toxic than 41N. Formulation 33T was far more toxic than the other formulations and omitted from further analysis, while 28M was similar in tolerability to 37N with significantly lower potency. A direct comparison of the potency vs. tolerability (weighted equally) indicated that formulation 41N scored higher than 37N, which in turn scored significantly higher than 28M and 33T (FIG. 16e). The tolerability results obtained with FAST-PLV formulations in Vero cells were confirmed in vivo by intravenous injection of mice with an 8 mg/kg dose of pDNA-FLuc, using pro-inflammatory cytokine responses (TNF-α, IL-6, IFN-γ, IL-1β, CXCL1, IL-10, IL-5) as an indicator of immunotoxicity (56). Mice injected with FAST-PLV formulation 41N induced the lowest levels of pro-inflammatory cytokines, comparable to those in the PBS-injected control mice (FIG. 16f-i). Formulation 37N induced modest, but consistently higher, increases in several cytokines compared to 41N, while formulation 28M induced significant elevations in all cytokine levels (FIG. 16f-i). These in vivo tolerability results mirror those observed in Vero cells. A direct comparison of the efficacy of 41N and 37N PLVs containing pDNA-FLuc was then performed in immune-competent animals through the intramuscular route. In contrast to the in vitro potency, it was found that 41N PLVs significantly outperformed 37N, by a factor of almost 10 (FIG. 16m, n). Without wishing to bound by theory, this is likely attributable to lower localized toxicity and immune stimulation which facilitated an improvement in pDNA delivery. Based on all these results, PLV formulation 41N was selected for further development and optimization.

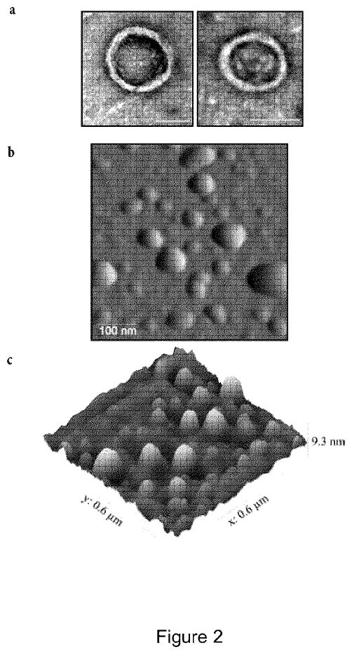

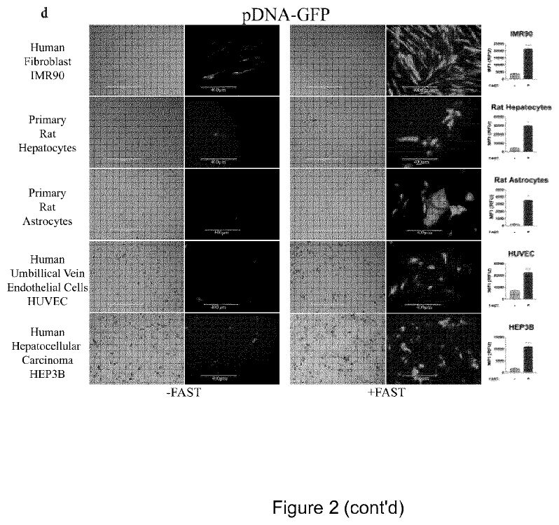

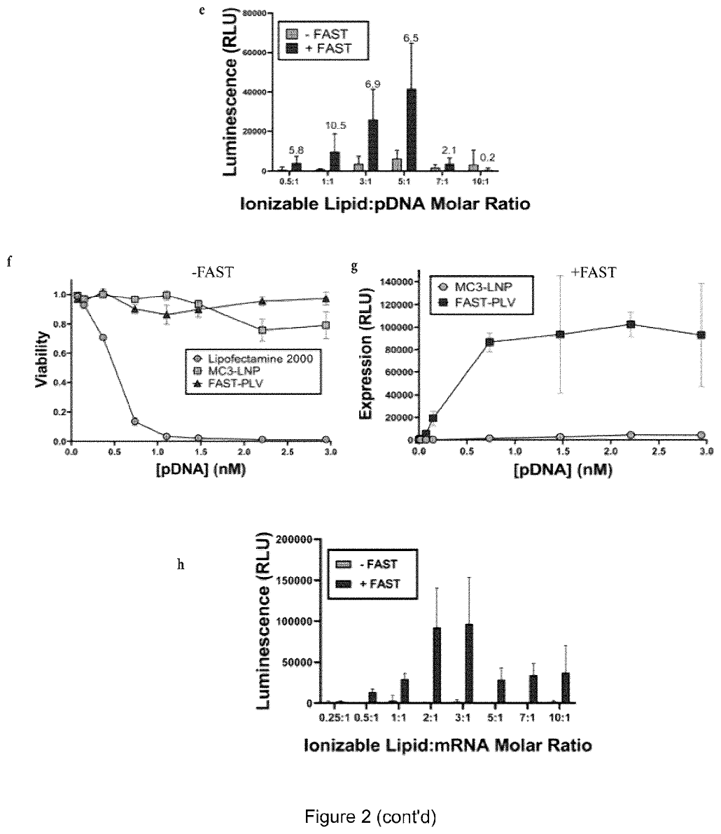

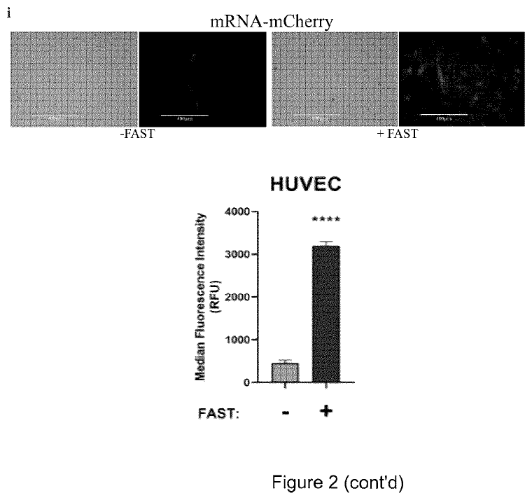

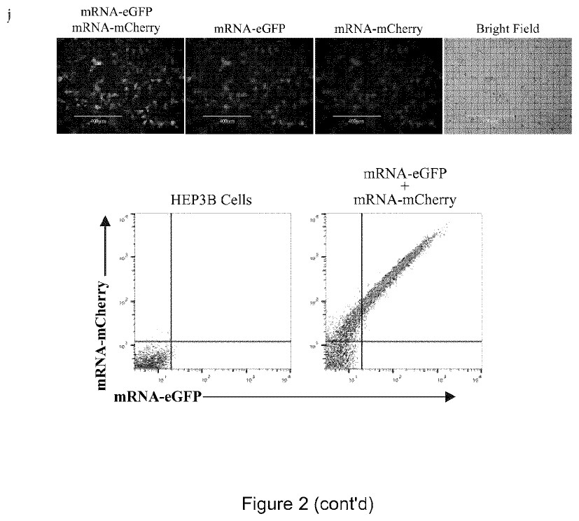

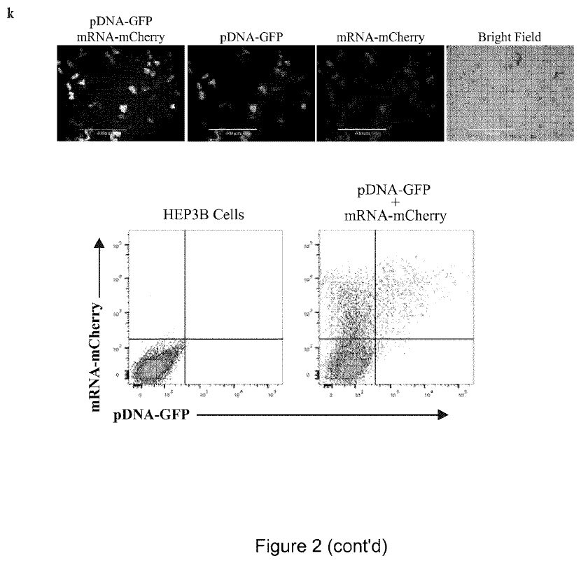

The size and structure of the 41N pDNA FAST-PLV was assessed by transmission electron microscopy (TEM) and atomic force microscopy (AFM). TEM revealed uniform spherical structures made of an outer lipid bilayer and a negatively stained inner core (FIG. 2a). AFM showed uniform spherical structures with an approximate size distribution of 66.3±15.3 nm (FIGS. 2b, c), in agreement with the dynamic light scattering (DLS) measurements (Table 16). The potency of 41N pDNA-GFP PLV formulated with and without FAST protein and with varying ratios of ionizable lipid to pDNA-GFP was evaluated in a panel of cultured and primary cell lines. In all cell lines tested, FAST-containing formulations showed significantly improved potency as measured by GFP expression (FIG. 2d), which was maximal at a 5:1 ratio of ionizable lipid to pDNA (FIG. 2e). Based on this, the optimized FAST-PLV formulation was finalized using the 41N lipid formulation (DODAP, DOPE, and DMG-PEG lipids in a molar ratio of 66:30:4) and a 5:1 molar ratio of DODAP to pDNA.

To establish a suitable reference point, the potency and tolerability of FAST-PLVs to the cationic lipid formulation Lipofectamine 2000 and a conventional LNP formulation composed of DLin-MC3-DMA (MC3-LNPs) was compared in human umbilical vein endothelial cells (HUVEC) and human fibroblasts (IMR-90) (23). FAST-PLVs containing pDNA-FLuc were significantly less toxic than Lipofectamine 2000, with comparable tolerability to MC3-LNPs (FIG. 2f). However, FAST-PLVs demonstrated significantly higher expression of pDNA-FLuc than MC3-LNPs (FIG. 2g).

The delivery of mRNA in the 41N PLV formulation with and without FAST protein was then assessed using a range of molar ratios of DODAP to mRNA. The highest expression of mRNA-FLuc in WI-38 cells was found at a DODAP:mRNA ratio of 3:1, and the incorporation of FAST protein significantly enhanced potency in every case (FIG. 2h). Similar results were also seen with the delivery of mRNA-mCherry into human primary endothelial cells (HUVEC) using 41N PLVs (FIG. 2i). The ability of FAST-PLVs to deliver multiple mRNAs to the same cell was demonstrated by co-expression of mRNA-mCherry and mRNA-eGFP in cells treated with PLVs containing a 1:1 molar ratio of each mRNA at a combined 12 nM dose (FIG. 2j). Furthermore, FAST-PLVs formulated with a mixture of pDNA-GFP and mRNA-mCherry payload at a 1:6 molar ratio demonstrated expression of both reporters in cells at a combined 7 nM dose (FIG. 2k). Thus, the optimized 41N FAST-PLV formulation is suitable for encapsulation and intracellular delivery of pDNA and/or mRNA to cultured and primary cells with high potency and low toxicity compared to conventional LNPs.

### Example 3: Determination of Systemic, In Vivo Delivery of pDNA and mRNA by p14endo15-PLV

As p14endo15-PLV was able to effectively deliver pDNA, mRNA, as well as combinations of two different mRNA cargos, or pDNA plus mRNA, for expression in vitro, the delivery and expression efficiency of p14endo15-PLV encapsulating mRNA-FLuc in vivo was examined. Safe and effective systemic delivery of DNA is a significant challenge, with many platforms exhibiting low tolerability in vivo leading to host immune stimulation and liver toxicity (41, 57, 58). The in vivo biodistribution, potency and tolerability of FAST-PLVs was evaluated compared to the conventional MC3-LNP lipid formulation over a dose range of 0.5 mg/kg to 80 mg/kg DNA. Systemic intravenous administration of MC3-LNPs encapsulating pDNA-FLuc at doses higher than 1 mg/kg resulted in significant mortality of mice within 48 hours of injection while FAST-PLVs were well tolerated at doses>60 mg/kg (Tables 3-5).

TABLE 3
The tolerability and survivability of mice injected with
a range of dose concentrations of p14endo15-PLVs encapsulating
pDNA versus MC3-LNP encapsulating pDNA.

| | p14endo15-PLV | | MC3-LAP | |
|---|---|---|---|---|
| Dose | Total Mice | Surviving | Total Mice | Surviving |
| (mg/kg) | Injected | Mice | Injected | Mice |
| 0.5 | ND | | 3 | 3 |
| 1 | 3 | 3 | 3 | 1 |
| 5 | 3 | 3 | 3 | 0 |
| 8 | 3 | 3 | 3 | 0 |

| | | | |
|---|---|---|---|
| 20 | 3 | 3 | ND |
| 60 | 3 | 3 | ND |
| 80 | 3 | 2 | ND |

TABLE 4
Maximum tolerated pDNA-FLuc dose when encapsulated
within Formulation 41 (p14e15-PLV)

| p14e15-PLV Dose: | | Surviving Mice | Total Mice Injected |
|---|---|---|---|
| 5 | mg/kg | 3 | 3 |
| 6.5 | mg/kg | 2 | 2 |
| 20 | mg/kg | 6 | 6 |
| 60 | mg/kg | 1 | 1 |
| 80 | mg/kg | 1 | 1 |

TABLE 5
Maximum tolerated pDNA-FLuc dose
when encapsulated within MC3

| MC3 Dose: | | Surviving Mice | Total Mice Injected |
|---|---|---|---|
| 0.5 | mg/kg | 3 | 3 |
| 1 | mg/kg | 2 | 3 |
| 1.5 | mg/kg | 0 | 1 |
| 5 | mg/kg | 0 | 1 |
| 8 | mg/kg | 0 | 2 |

All mice injected with Formulation 41 survive, whereas mice receiving doses greater than 1 mg/kg MC3 were subject to severe morbidity and mortality.

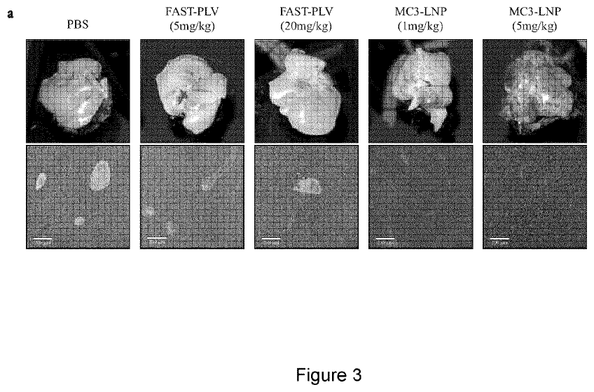

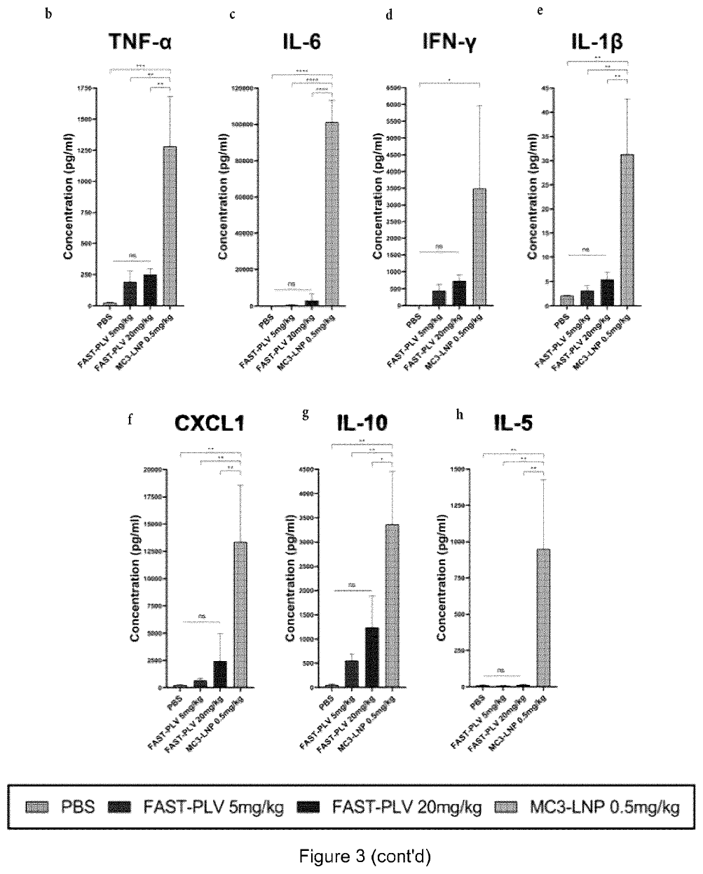

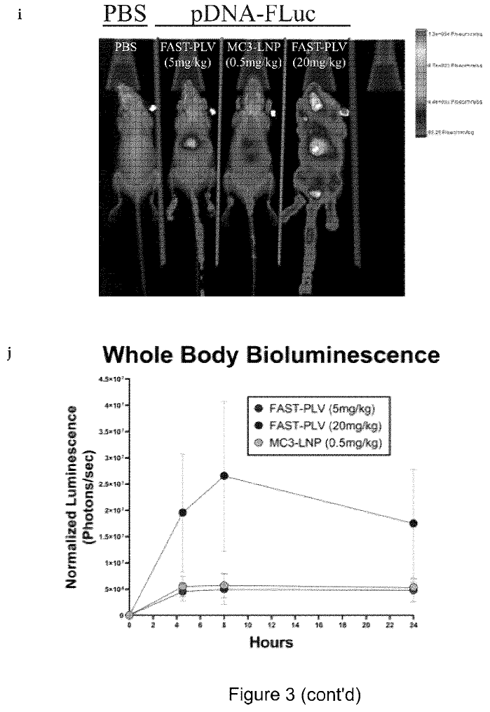

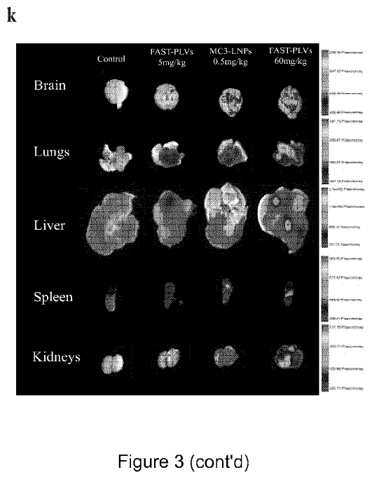

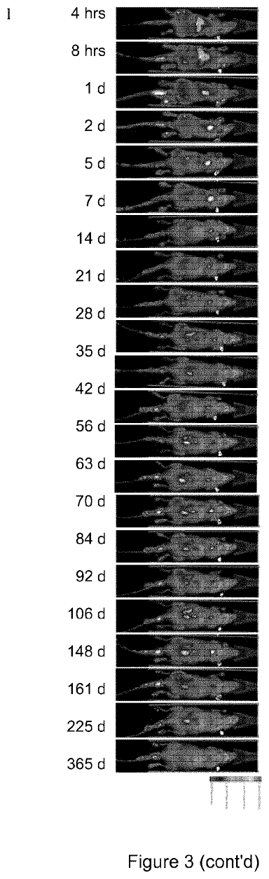

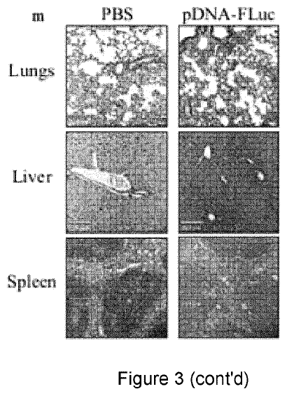

Post-mortem analysis of MC3-LNP treated mice at 1 and 5 mg/kg revealed significant liver toxicity that was readily apparent upon gross visual examination (FIG. 3a), with histological analysis indicating liver damage characterized by hemorrhage (FIG. 3b). In contrast, mice treated with FAST-PLVs at doses up to 20 mg/kg dose showed no signs of liver pathology by gross visual or histological examination (FIGS. 3a, b). Cytokine responses in mice were also examined as an immune indicator of toxicity following intravenous injection of FAST-PLVs administered at 5 mg/kg and 20 mg/kg pDNA doses or MC3-LNPs administered at a 0.5 mg/kg dose pDNA, a typical dose for systemic delivery of mRNA (23). Mice receiving either dose of FAST-PLVs encapsulating pDNA-FLuc showed no significant increase in any of the examined cytokines while all cytokines, particularly IL-6, TNF-α, and IL-5, were significantly elevated in mice treated with the MC3-LNPs (FIGS. 3c-i).

Whole-body luminescence imaging was used to evaluate the in vivo delivery capability of FAST-PLVs. Results showed a dose-dependent increase in FLuc expression between the 5 and 20 mg/kg FAST-PLV doses, with the 5 mg/kg dose of FAST-PLVs generating comparable signals to the 0.5 mg/kg dose of MC3-LNPs (FIG. 3j, k), while ex vivo bioluminescence showed 5 mg/kg FAST-PLVs generated comparable FLuc signals in multiple organs to 0.5 mg/kg MC3-LNPs (FIG. 3l). The durability of in vivo pDNA expression was also assessed in immune-competent mice by whole-body luminescence imaging over 365 days after systemic administration of 20 mg/kg pDNA-FLuc FAST-PLVs. A strong and widespread whole-body luminescence signal was observed in the first two days that decreased to a steady state maintained for greater than twenty weeks, with expression continuing through one year after administration (FIG. 3m). Immunohistochemistry of the lungs, liver, and spleen of animals 63 days after injection demonstrated widespread expression of FLuc in each tissue examined (FIG. 3m).

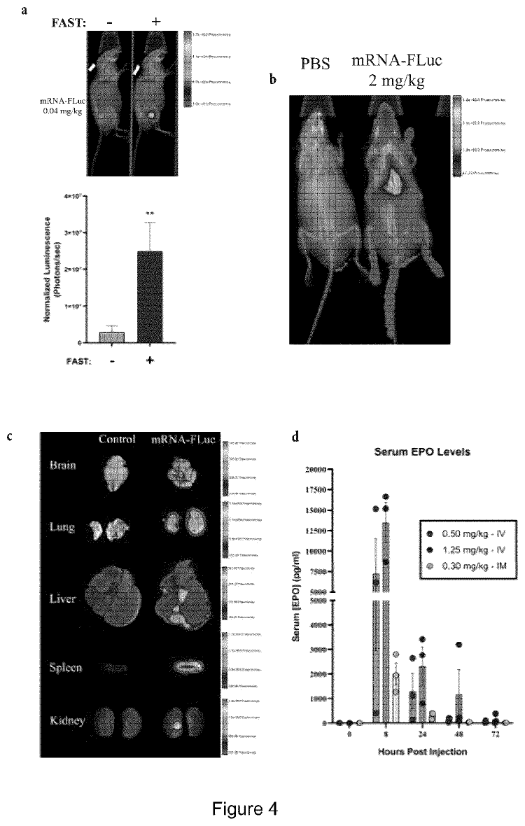

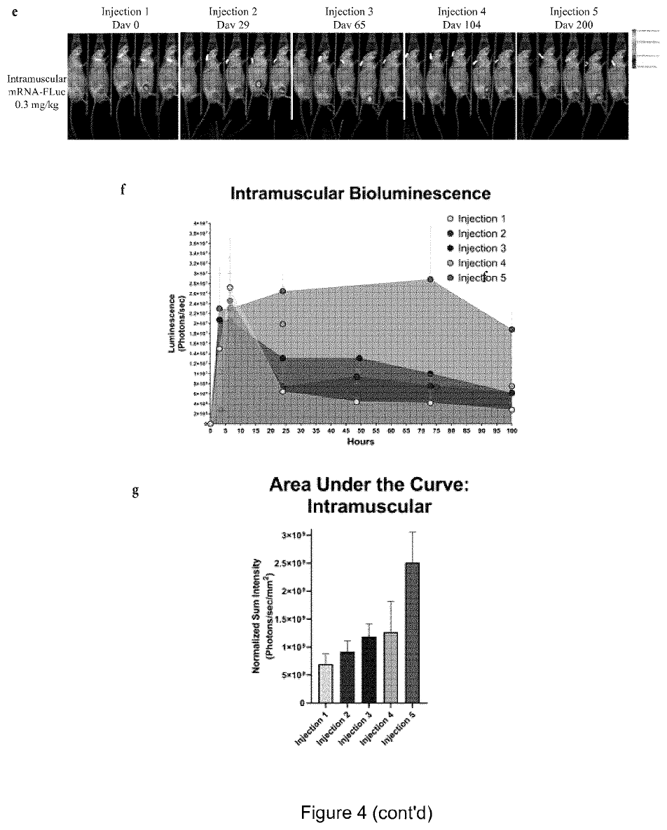

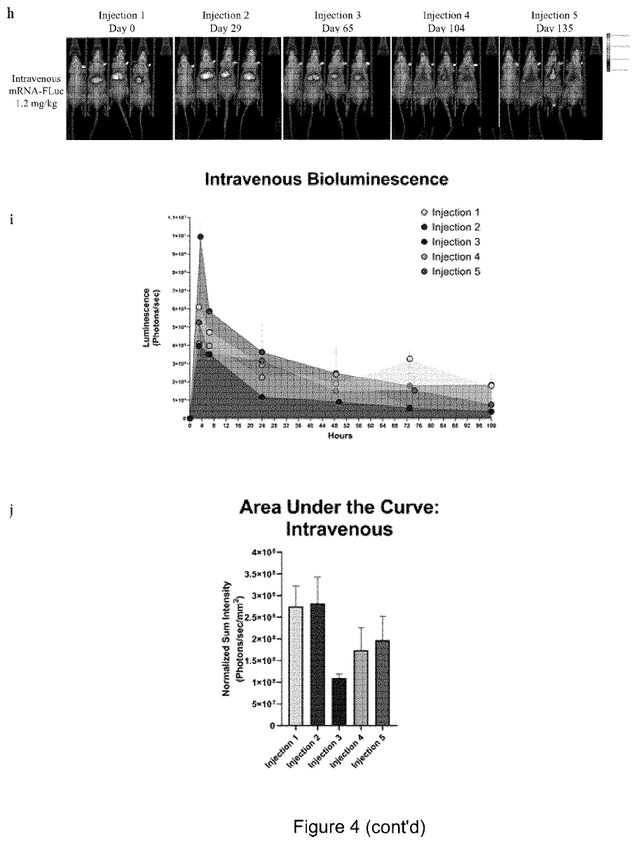

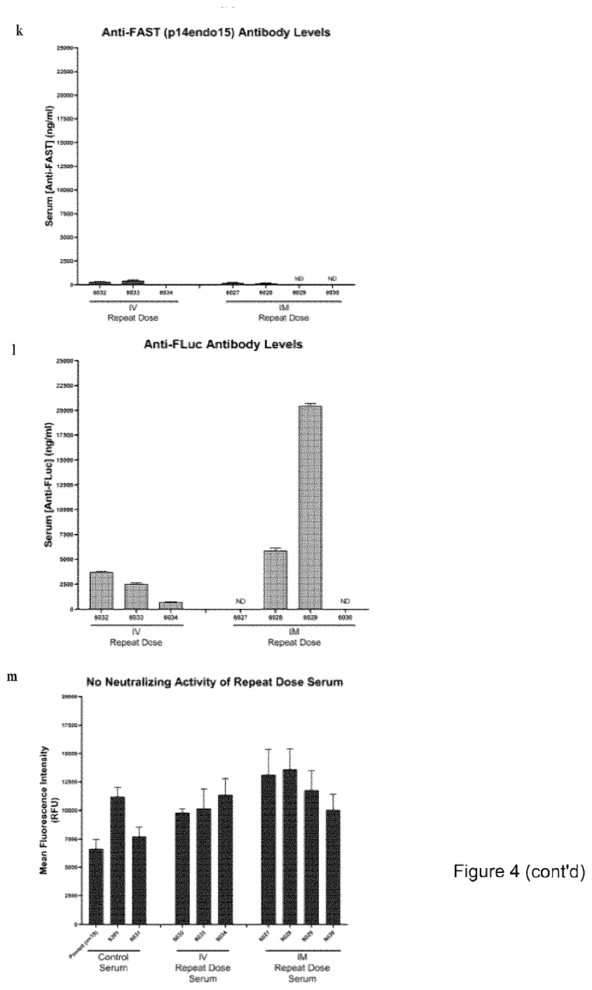

Next, the ability of FAST-PLVs to deliver mRNA in vivo was assessed. Incorporation of FAST protein into mRNA-FLuc PLVs resulted in significantly increased expression following intramuscular injection (FIG. 4a). Widespread whole-body luminescence was observed four hours after intravenous injection with FAST-PLVs encapsulating mRNA-FLuc at a dose of 2 mg/kg (FIG. 4b), and ex vivo luminescence imaging confirmed luciferase expression in a wide array of organs, including the lungs, liver, spleen, kidneys, and brain (FIG. 4c). Next, the ability of FAST-PLVs to deliver therapeutic mRNA cargos was examined. Delivery of LNPs encapsulating human erythropoietin (EPO) encoding mRNA is currently being investigated by multiple research groups as a potential anemia treatment modality (13, 17, 25, 59). As such, circulating EPO levels in mice following intravenous injection with FAST-PLV encapsulating 0.5 or 1.25 mg/kg mRNA-EPO, as well as intramuscular injection with FAST-PLVs encapsulating 0.3 mg/kg mRNA-EPO was investigated. 8 hours after injection, a large spike in serum EPO levels following systemic FAST-PLV injection reaching 7000 μg/ml and 13000 μg/ml was observed in mice injected with the low and high dose mRNA-EPO, respectively. Surprisingly, intramuscular injection with only 0.3 mg/kg mRNA-EPO resulted in a detectable serum EPO concentration just under 2000 μg/ml 8 hours following injection. Circulating EPO levels returned to near baseline levels 48 hours after injection for low systemic dose and intramuscularly injected mice, and 72 hours for high systemic dose mice (FIG. 4d). Overall, systemic in vivo administration of FAST-PLVs encapsulating either pDNA or mRNA showed durable, dose-dependent expression and biodistribution to a wide array of organs with no significant increase in liver or immune-toxicity at doses significantly higher than can be achieved with conventional LNPs.

### Example 4: Repeat Dosing of FAST-PLVs (p14endo15) Through Intramuscular or Systemic Route does not Generate Significant Immunogenic Responses

Administered biologic drugs such as monoclonal antibodies or viral gene therapy vectors elicit an adaptive immune response in the form of anti-drug or anti-vector antibodies that can interfere with or neutralize the effect of the drug, restricting their use in applications that require repeat dosing (5, 6, 8). Given the incorporation of a novel virus-derived fusion protein in the current FAST-PLV platform, experiments were conducted to determine if FAST-PLVs generate an immune response capable of reducing their in vivo efficiency upon repeated administration intramuscularly or intravenously. To that end, FAST-PLVs encapsulating mRNA-FLuc were administered at 0.3 mg/kg via intramuscular injection (FIG. 4e-g) or 1.2 mg/kg intravenously (FIG. 4h-j) once per month for six months in mice. Significant expression of luciferase was seen after each intramuscular injection (FIG. 4e), with a slight increase in expression observed over each injection, likely attributable to residual expression from the previous doses accumulating over time (FIG. 4f, g). When FAST-PLVs were repeatedly administered intravenously, no significant change in total luminescence intensity was observed over time (FIG. 4h-j). Antibodies against FAST protein were quantified in the serum of these mice one month after the fifth and final FAST-PLV administration using indirect ELISA with a lower limit of quantification (LLOQ) of 50 ng/mL. After intramuscular administration, two of the four animals had no detectable anti-FAST antibody level and two had levels just above the LLOD at 221.6±40.6 and 173.5±36.3 ng/mL. Of the three mice repeatedly administered FAST-PLVs intravenously, one had no detectable anti-FAST antibody while two had levels of 335.2±5.1 and 453.7±71.1 ng/mL (FIG. 4k). Interestingly, high titers of anti-FLuc antibodies were detected in all intravenously repeat-dosed animals and two out of four intramuscular repeat-dosed animals (FIG. 4l), which may help to explain the variability in luciferase expression following systemic repeat doses (FIG. 4h). To determine whether the detected levels anti-FAST antibodies have neutralizing activity, the activity of pDNA-GFP PLVs in the presence of serum from repeatedly dosed and control mice was assessed in an in vitro transfection assay. No reduction in GFP expression was observed when serum treated FAST-PLVs were used to transfect 3T3 cells in vitro (FIG. 4m), indicating no neutralizing activity. It was determined that repeated intramuscular or intravenous injections of p14endo15-PLVs encapsulating two different doses of mRNA-FLuc into mice produced consistent reporter expression, was well tolerated, and did not stimulate adverse immune responses making this delivery platform highly suitable for repeat dose gene therapies.

### Example 5: FAST-PLVs Safely and Effectively Deliver pDNA in Non-Human Primates with Broad Biodistribution to Tissues

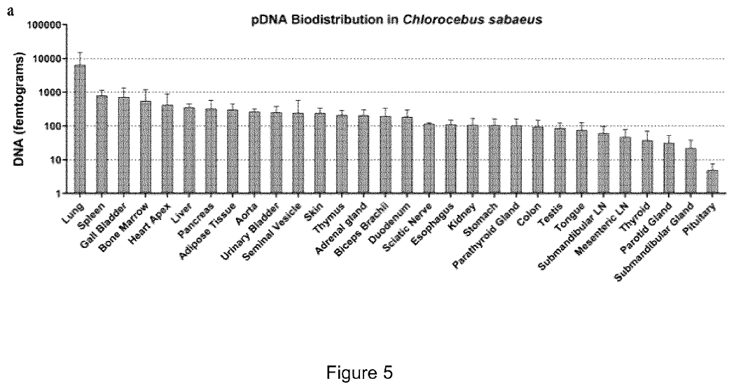

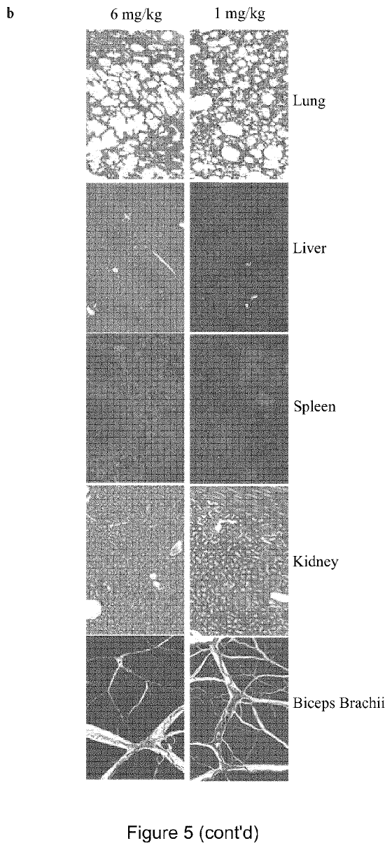

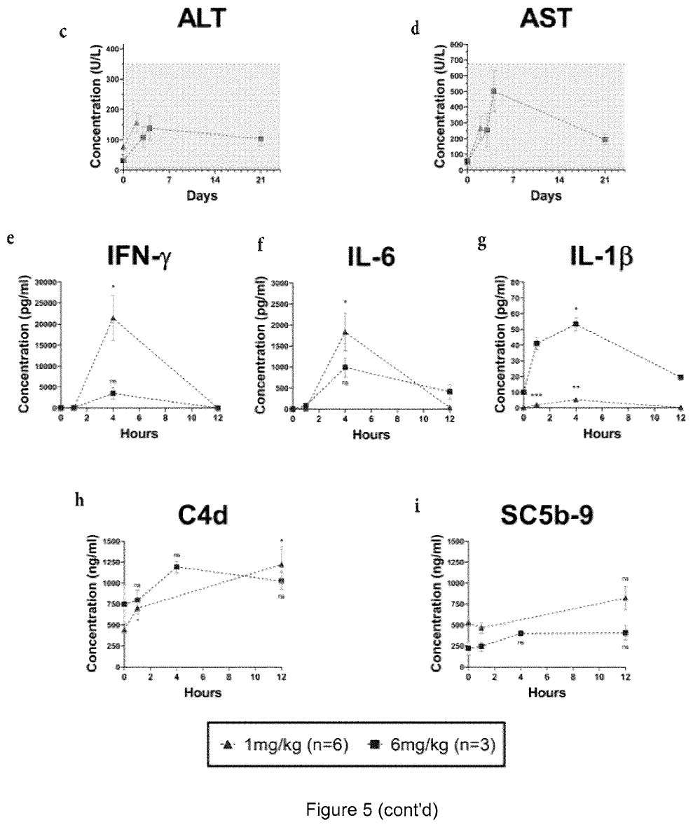

The safety, tolerability and biodistribution of FAST-PLVs at various doses following systemic administration was further examined in African green monkeys (Chlorocebus sabaeus). The biodistribution of pDNA-GFP cargo delivered by intravenous administration in FAST-PLV at a 1 mg/kg dose of pDNA was quantified in 30 tissues using a quantitative PCR approach two days after administration. Significant levels of pDNA were detected in all organs tested, with the highest levels detected in the lungs, spleen, gall bladder, and bone marrow. Of particular interest was the fact that liver ranked sixth for pDNA accumulation, which, unlike conventional LNP, suggests that extrahepatic pDNA delivery can be achieved (FIG. 5a). Tolerability was similarly evaluated following intravenous dosing with FAST-PLVs at 1 and 6 mg/kg pDNA doses. No significant abnormalities were observed in any organs assessed (FIG. 5b), and no treatment-related changes were noted in any organs tested (Table 6).

TABLE 6
Tissue pathology from the nonhuman primate study of FAST-PLV
carrying plasmid DNA at two separate doses; 1 and 6 mg/kg.

| Organ | Feature | 1 mg/kg | 6 mg/kg |
|---|---|---|---|
| Lung | Alveolar Macrophages | 0 (3/3) | 1 (2/3), 2 (1/3) |
| Liver | Mononuclear Cell Infiltration | 1 (3/3) | 1 (3/3) |
| | Sinusoidal Leukocytes | 0 (3/3) | 0 (3/3) |
| | Vacuolation | 0 (3/3) | 0 (3/3) |
| | Biliary Hyperplasia | 0 (3/3) | 0 (3/3) |
| | Periportal Hemosiderin | 0 (2/3), 1 (1/3) | 0 (3/3) |
| Spleen | Congestion | 0 (1/3), 2 (1/3), | 0 (3/3) |
| | | 3 (1/3) | |
| | Decreased Cellularity, | 0 (2/3), 3 (1/3) | 0 (3/3) |
| | Red Pulp | | |
| Heart | Mononuclear Cell Infiltration | 0 (3/3) | 0 (3/3) |
| | Fibrosis | 0 (3/3) | 0 (3/3) |
| Kidney | Glomerulopathy | 0 (3/3) | 0 (3/3) |

All pathological findings are scored on a scale from 0 to 5: 0 = No Visible Lesions, 1 = Minimal, 2 = Mild, 3 = Moderate, 4 = Marked, 5 = Severe.

The first number denotes the grade of lesion being reported; the second number in brackets denotes the number of subjects with the finding/total number of subjects evaluated.

Some findings were reported in all animals in the aged, feral African green monkey cohort used for the study. For example, the mononuclear cell infiltrates observed in the livers of all animals, as well as the vacuolation and hydropic degeneration (consistent with increased hepatocellular glycogen) were within normal variability for African green monkeys. Additionally, the periportal hemosiderin and pigmented macrophages detected are typical of prior parasite migration tracks—consistent with the use of wild-caught animals (60, 61). Circulating levels of alanine transaminase (ALT) and aspartate transaminase (AST) remained within the normal range for the duration of the study (FIG. 5c, d). Creatine phosphokinase (CPK) and lactate dehydrogenase (LDH) levels were elevated immediately following infusion but decreased to within expected ranges by 21 days post infusion. All other clinical chemistry parameters remained within the normal range for the study duration (Table 7).

TABLE 7
Clinical chemistry composition in non-human primates.

| | | | PLV | | | | | | |
|---|---|---|---|---|---|---|---|---|---|
| | Normal | | Dose | Time Post Infusion (Days) | | | | | |
| Parameter | Range | Units | (mg/kg) | 0 | 2 | 3 | 4 | 14 | 21 |

| | | | | | | | | |
|---|---|---|---|---|---|---|---|---|
| Blood urea | 6.3-31.3 | mg/dL | 1 | 18.67 ± | 16.33 ± | | | |
| nitrogen (BUN) | | | | 5.03 | 0.58 | | | |
| | | | 6 | 18.33 ± | | 20.33 ± | 29.33 ± | 23.00 ± |
| | | | | 2.52 | | 1.15 | 2.31 | 2.65 |
| Creatinine | 0.3-1.3 | mg/dL | 1 | 0.80 ± | 0.80 ± | | | |
| | | | | 0.10 | 0.10 | | | |
| | | | 6 | 0.77 ± | | 0.93 ± | 1.03 ± | 0.77 ± |
| | | | | 0.06 | | 0.15 | 0.06 | 0.06 |
| Glucose | 32.5-199.1 | mg/dL | 1 | 109.67 ± | 185.33 ± | | | |
| | | | | 37.31 | 17.62 | | | |
| | | | 6 | 165.00 ± | | 144.00 ± | 124.33 ± | 203.00 ± |
| | | | | 69.35 | | 41.07 | 6.03 | 38.94 |
| Sodium (Na) | 141.7-154.8 | mmol/L | 1 | 150.33 ± | 147.00 ± | | | |
| | | | | 3.21 | 2.65 | | | |
| | | | 6 | 147.67 ± | | 147.00 ± | 145.67 ± | 147.67 ± |
| | | | | 4.73 | | 1.00 | 0.58 | 2.31 |
| Potassium (K) | 2.6-5.4 | mmol/L | 1 | 3.97 ± | 3.6 ± | | | |
| | | | | 0.15 | 0.36 | | | |
| | | | 6 | 3.93 ± | | 3.67 ± | 3.70 ± | 4.40 ± |
| | | | | 0.38 | | 0.12 | 0.10 | 0.20 |
| Chloride (Cl) | 100.6-112.5 | mmol/L | 1 | 108.67 ± | 107 ± | | | |
| | | | | 2.08 | 1.73 | | | |
| | | | 6 | 105.67 ± | | 104.67 ± | 104.00 ± | 102.00 ± |
| | | | | 3.06 | | 1.53 | 1.00 | 1.73 |
| Alkaline | 25-360.5 | U/L | 1 | 117.00 ± | 106.67 ± | | | |
| phosphatase (ALP) | | | | 81.43 | 53.98 | | | |
| | | | 6 | 103.33 ± | | 108.33 ± | 102.67 ± | 108.67 ± |
| | | | | 21.22 | | 42.34 | 41.86 | 47.72 |
| Total bilirubin | 0.1-1.2 | mg/dL | 1 | 0.20 ± | 0.40 ± | | | |
| | | | | 0.10 | 0.20 | | | |
| | | | 6 | 0.20 ± | | 0.33 ± | 0.40 ± | 0.30 ± |
| | | | | 0.00 | | 0.15 | 0.10 | 0.00 |
| Lactate | 53.5-622.5 | U/L | 1 | 442.00 ± | 798.00 ± | | | |
| dehydrogenase (LDH) | | | | 404.73 | 213.42 | | | |
| | | | 6 | 309.67 ± | | 1022.33 ± | 2394.00 ± | 698.67 ± |
| | | | | 182.15 | | 970.50 | 1511.01 | 51.54 |
| Creatine | 193-4843.1 | U/L | 1 | 1545.33 ± | 22717.00 ± | | | |
| phosphokinase (CPK) | | | | 1750.30 | 9336.12 | | | |
| | | | 6 | 2214.33 ± | | 18988.33 ± | 47630.33 ± | 14367.33 ± |
| | | | | 2616.93 | | 17330.98 | 25298.93 | 5572.00 |
| Gamma glutamyl | 3.6-96.7 | U/L | 1 | 32 ± | 41.67 ± | | | |
| transferase (GGT) | | | | 16.70 | 15.01 | | | |
| | | | 6 | 43.00 ± | | 44.00 ± | 42.00 ± | 53.00 ± |
| | | | | 23.90 | | 16.52 | 15.13 | 13.86 |
| Total protein | 5.8-8.3 | g/dL | 1 | 5.40 ± | 6.00 ± | | | |
| | | | | 0.72 | 0.44 | | | |
| | | | 6 | 6.57 ± | | 6.80 ± | 6.67 ± | 6.87 ± |
| | | | | 1.24 | | 0.35 | 0.40 | 0.29 |
| Albumin | 3.4-5.6 | g/dL | 1 | 3.60 ± | 3.83 ± | | | |
| | | | | 0.60 | 0.49 | | | |
| | | | 6 | 4.17 ± | | 4.27 ± | 4.27 ± | 4.20 ± |
| | | | | 0.72 | | 0.15 | 0.21 | 0.10 |
| Globulin | 1.4-3.8 | g/dL | 1 | 1.80 ± | 2.17 ± | | | |
| | | | | 0.20 | 0.12 | | | |
| | | | 6 | 2.40 ± | | 2.53 ± | 2.40 ± | 2.67 ± |
| | | | | 0.53 | | 0.21 | 0.20 | 0.31 |
| Albumin/Globulin | 0.8-2.7 | Ratio | 1 | 2.00 ± | 1.78 ± | | | |
| ratio | | | | 0.29 | 0.30 | | | |
| | | | 6 | 1.75 ± | | 1.69 ± | 1.78 ± | 1.59 ± |
| | | | | 0.13 | | 0.10 | 0.07 | 0.20 |
| Calcium (Ca) | 8.1-10.3 | mg/dL | 1 | 8.80 ± | 8.73 ± | | | |
| | | | | 0.26 | 0.58 | | | |
| | | | 6 | 9.33 ± | | 9.23 ± | 9.57 ± | 9.90 ± |
| | | | | 0.92 | | 0.06 | 0.51 | 0.35 |
| Phosphorus | 2.0-8.0 | mg/dL | 1 | 3.83 ± | 5.93 ± | | | |
| | | | | 1.84 | 1.36 | | | |
| | | | 6 | 5.47 ± | | 4.93 ± | 4.17 ± | 4.73 ± |
| | | | | 1.56 | | 0.55 | 0.70 | 1.10 |
| Cholesterol | 88.4-176.2 | mg/dL | 1 | 102.00 ± | 91.67 ± | | | |
| | | | | 8.19 | 20.31 | | | |
| | | | 6 | 117.33 ± | | 152.67 ± | 152.33 ± | 160.67 ± |
| | | | | 21.46 | | 29.96 | 34.02 | 16.29 |
| Triglycerides | 1.9-105.9 | mg/dL | 1 | 37.33 ± | 64.00 ± | | | |
| | | | | 11.37 | 10.15 | | | |
| | | | 6 | 40.33 ± | | 34.67 ± | 52.33 ± | 69.00 ± |
| | | | | 2.52 | | 9.29 | 3.51 | 39.28 |
| Glutamate | 3-42 | U/L | 1 | 36.67 ± | 101.33 ± | | | |
| dehydrogenase (GDH) | | | | 16.80 | 40.5 | | | |
| | | | 6 | 13.33 ± | | 20.67 ± | 17.33 ± | 35.33 ± |
| | | | | 5.13 | | 11.50 | 6.35 | 25.17 |

A transient elevation in systemic pro-inflammatory cytokines was observed 1-4 hours after FAST-PLV infusion that returned to baseline levels 12-72 hours post-infusion (FIG. 5e-g and Table 8).

TABLE 8
Cytokine composition in non-human primates.

| | PLV | | | | | |
|---|---|---|---|---|---|---|
| Cytokine | Dose | Time Post Infusion (Hours) | | | | |
| (pg/ml) | (mg/kg) | 0 | 1 | 4 | 12 | 72 |
| IL-2 | 1 | 0.03 ± 0.04 | 0.14 ± 0.08 | 1.57 ± 2.28 | 0.86 ± 0.96 | |
| | 6 | 14.65 ± 8.83 | 19.25 ± 1.63 | 24.21 ± 2.39 | 27.87 ± 12.92 | |
| IL-7 | 1 | 7.14 ± 1.95 | 8.37 ± 2.9 | 13.64 ± 3.32 | 6.91 ± 5.03 | |
| | 6 | 7.39 ± 4.84 | 11.05 ± 6.74 | 12.27 ± 6.58 | 10.97 ± 5.00 | 7.33 ± 4.29 |
| IL-8 | 1 | 290.72 ± 209.25 | 1055.34 ± 496.87 | 4683.95 ± 595.93 | 36.73 ± 37.68 | |
| | 6 | 841.14 ± 272.64 | 824.87 ± 231.92 | 2289.71 ± 121.99 | 1111.65 ± 264.04 | |
| IL-10 | 1 | 0.03 ± 0.02 | 0.27 ± 0.10 | 0.12 ± 0.18 | 0.09 ± 0.12 | |
| IL-12/ | 1 | 79.06 ± 30.17 | 92.07 ± 34.65 | 350.18 ± 134.45 | 69.53 ± 61.60 | |
| IL-23 p40 | 6 | 50.53 ± 69.79 | 79.15 ± 101.08 | 188.98 ± 251.27 | 92.37 ± 98.84 | 21.76 ± 22.09 |
| IL-15 | 1 | 1.76 ± 0.29 | 2.55 ± 0.41 | 4.83 ± 0.86 | 12.03 ± 9.51 | |
| | 6 | 2.49 ± 1.33 | 3.20 ± 1.16 | 3.99 ± 1.01 | 27.64 ± 6.49 | 4.07 ± 0.97 |
| IL-16 | 1 | 13.63 ± 4.93 | 618.94 ± 191.11 | 462.00 ± 146.09 | 140.90 ± 140.30 | |
| | 6 | 16.80 ± 12.70 | 597.54 ± 435.03 | 558.90 ± 351.72 | 170.96 ± 93.84 | 12.17 ± 5.19 |
| IL-17A | 1 | 13.85 ± 10.42 | 18.08 ± 13.83 | 65.79 ± 24.91 | 6.60 ± 6.28 | |
| | 6 | 8.57 ± 4.14 | 11.90 ± 7.09 | 26.53 ± 15.53 | 15.62 ± 7.63 | 9.54 ± 6.18 |
| GM-CSF | 1 | 0.06 ± 0.09 | 1.59 ± 0.66 | 3.36 ± 1.63 | 0.22 ± 0.22 | |
| | 6 | 0.04 ± 0.07 | 0.73 ± 0.39 | 1.76 ± 0.86 | 0.13 ± 0.13 | |
| TNF-β | 1 | 0.15 ± 0.13 | 0.40 ± 0.10 | 3.71 ± 2.92 | 5.93 ± 12.56 | |
| | 6 | 0.09 ± 0.09 | 0.14 ± 0.09 | 1.52 ± 0.92 | 0.54 ± 0.34 | 0.09 ± 0.08 |
| VEGF | 1 | 4.02 ± 3.60 | 0.52 ± 0.65 | 1.64 ± 1.91 | 15.83 ± 15.09 | |
| | 6 | 2.81 ± 1.44 | 3.53 ± 1.27 | 3.30 ± 3.11 | 12.99 ± 8.52 | 5.42 ± 4.94 |

A similar pattern was observed on chemokine secretion after FAST-PLV infusion, with chemokines returning to baseline values 72 hours after infusion (Table 9).

TABLE 9
Chemokine composition in non-human primates.

| | PLV | | | | | |
|---|---|---|---|---|---|---|
| Chemokine | Dose | Time Post Infusion (Hours) | | | | |
| (pg/ml) | (mg/kg) | 0 | 1 | 4 | 12 | 72 |
| Eotaxin | 1 | 48.57 ± 19.03 | 185.08 ± 95.42 | 136.19 ± 36.84 | 108.67 | |
| IP-10 | 1 | 222.87 ± 66.18 | 1098.04 ± 507.82 | 31159.32 ± 35453.74 | 16783.25 ± 16881.47 | |
| | 6 | 393.91 ± 83.67 | 653.69 ± 26.56 | 1315.36 ± 469.99 | 619.63 ± 130.70 | 186.64 ± 35.93 |
| MCP-1 | 1 | 138.83 ± 35.70 | 378.24 ± 142.42 | 4695.52 ± 180.44 | 4581.40 ± 125.88 | |
| | 6 | 1.86 ± 0.34 | 2.66 ± 0.84 | 5.61 ± 2.79 | 3.33 ± 1.00 | 2.15 ± 0.85 |
| MCP-4 | 1 | 6.93 ± 2.24 | 11.93 ± 5.51 | 67.15 ± 34.60 | 103.76 ± 49.91 | |
| | 6 | 3.70 ± 2.74 | 5.57 ± 1.26 | 6.00 ± 4.49 | 24.00 ± 1.54 | 3.76 ± 3.70 |
| MDC | 1 | 35.70 ± 10.99 | 53.79 ± 14.52 | 240.44 ± 103.96 | 109.95 ± 77.27 | |
| | 6 | 4.08 ± 1.10 | 5.61 ± 0.70 | 57.67 ± 14.78 | 20.63 ± 6.13 | 3.85 ± 0.33 |
| MIP-1α | 1 | 13.97 ± 12.78 | 228.60 ± 27.16 | 458.31 ± 108.78 | 89.19 ± 21.23 | |
| | 6 | 8.43 ± 1.80 | 9.94 ± 2.18 | 11.81 ± 1.97 | 73.60 ± 24.04 | 11.89 ± 2.12 |
| MIP-1β | 1 | 24.09 ± 3.91 | 1299.62 ± 148.57 | 1574.58 ± 267.97 | 426.80 ± 95.93 | |
| TARC | 1 | 7.60 ± 2.56 | 9.58 ± 2.56 | 13.34 ± 1.86 | 3.52 ± 0.69 | |
| | 6 | 35.20 ± 14.40 | 54.33 ± 16.03 | 64.05 ± 8.34 | 53.85 ± 9.07 | 32.51 ± 16.26 |

Elevations in cytokine and chemokines were not dose-dependent, suggesting factors related to the intravenous infusion, such as local inflammation, might be the principle contributing factor. Complement activation-related pseudoallergy (CARPA), which can cause potentially dangerous hypersensitivity reactions in response to intravenous injection of lipid-containing entities (33, 34), was also assessed. Serum levels of S protein bound C terminal complex (SC5b-9) did not increase significantly following FAST-PLV infusion (FIG. 5h), while serum levels of C4d were elevated approximately 3-fold by the 1 mg/kg dose but were unaffected by the 6 mg/kg dose (FIG. 5i). The lack of a dose dependent response indicates that it is unlikely the administration of FAST-PLVs is contributing to this elevation. (FIG. 5h, i).

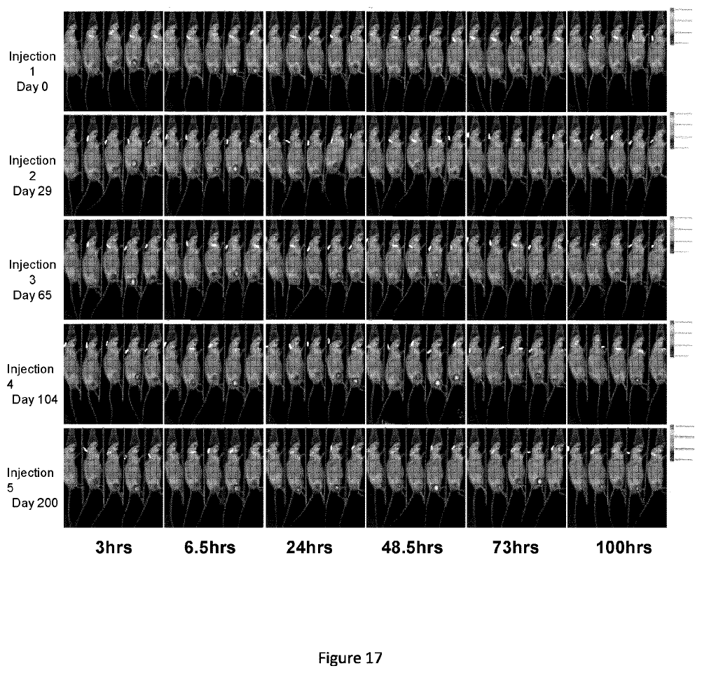

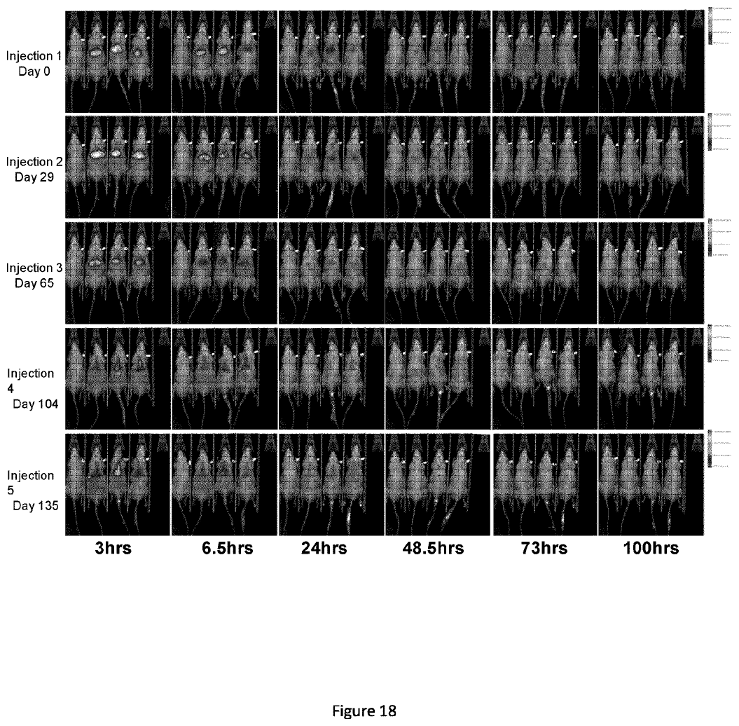

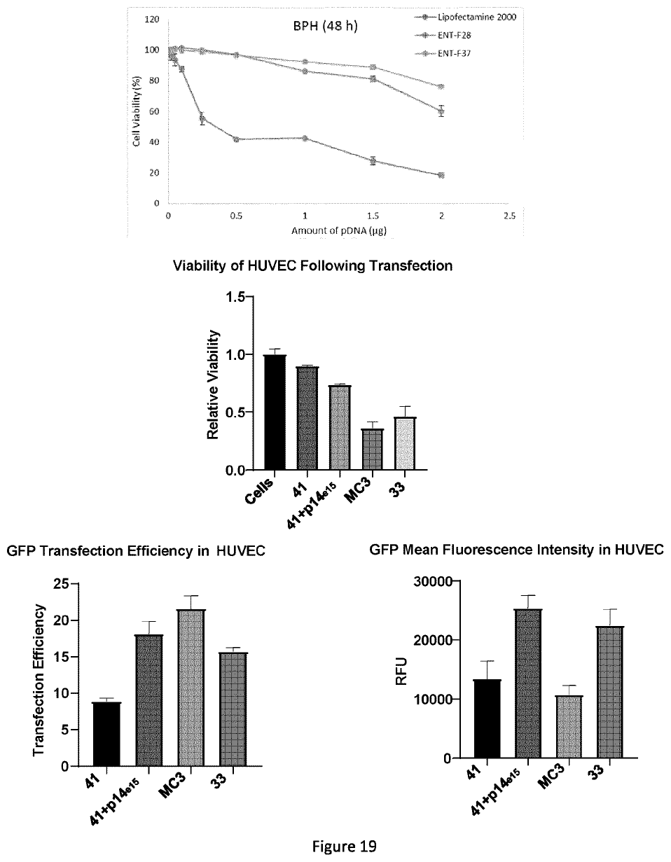

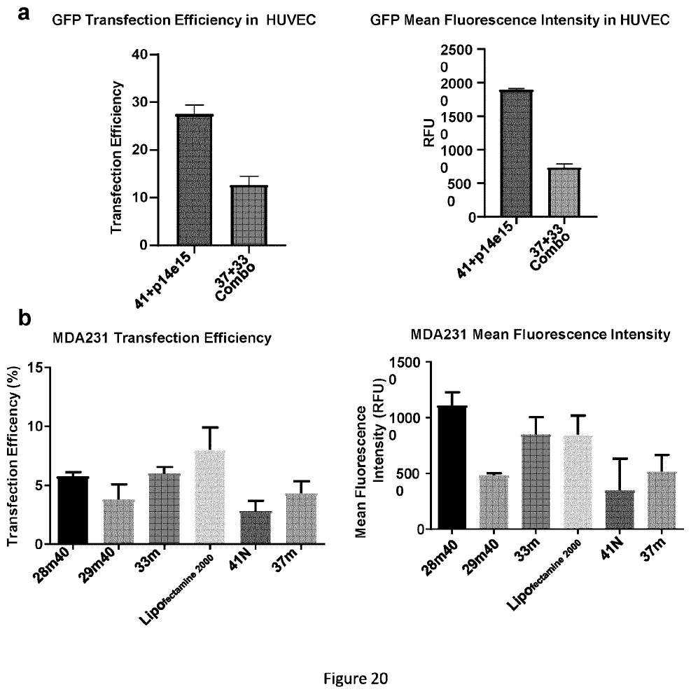

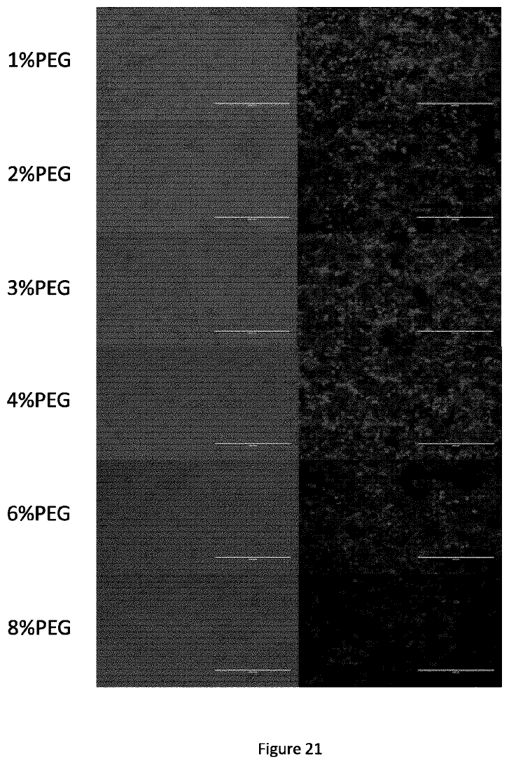

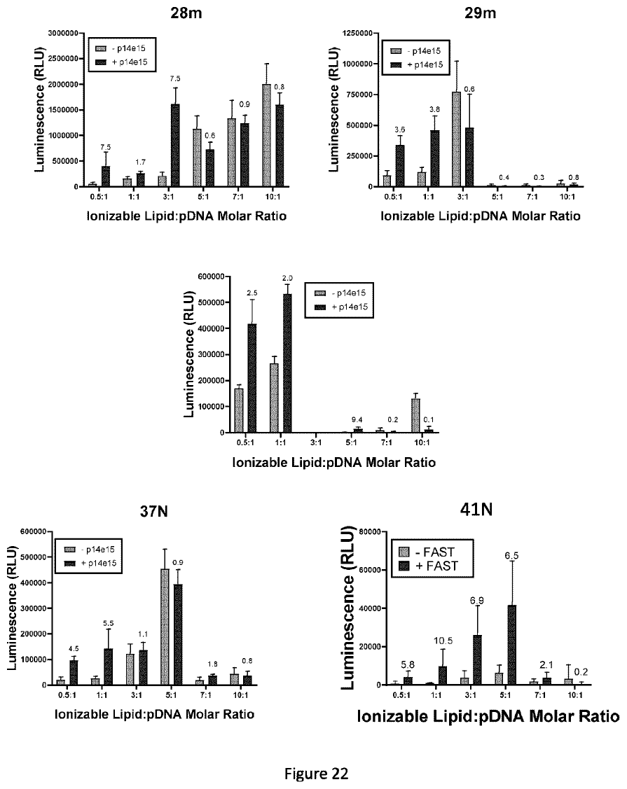

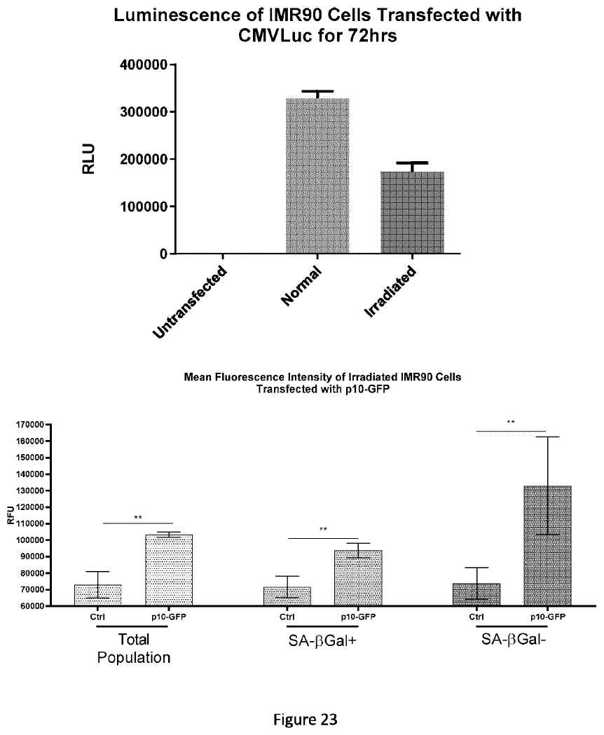

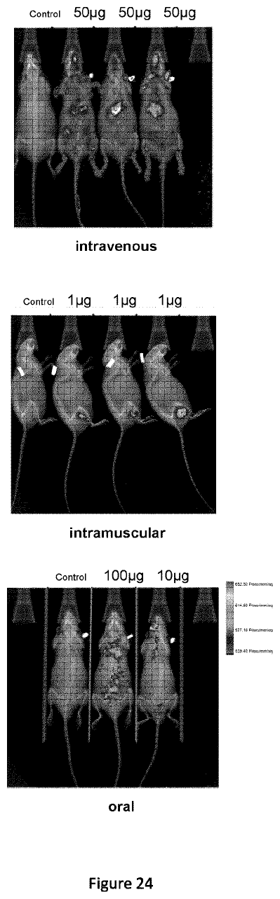

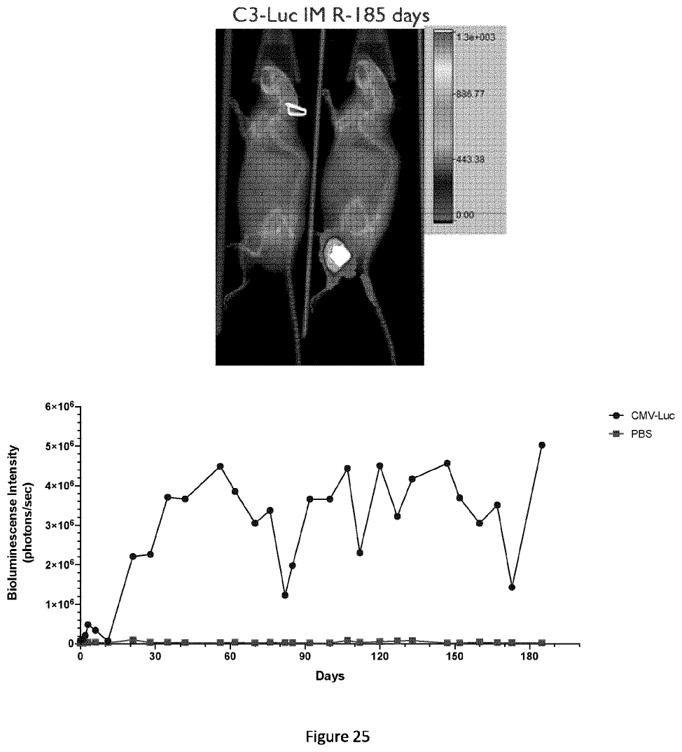

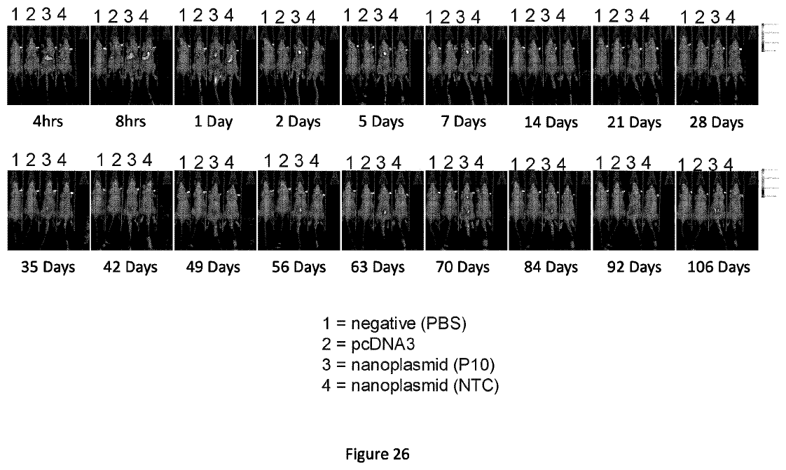

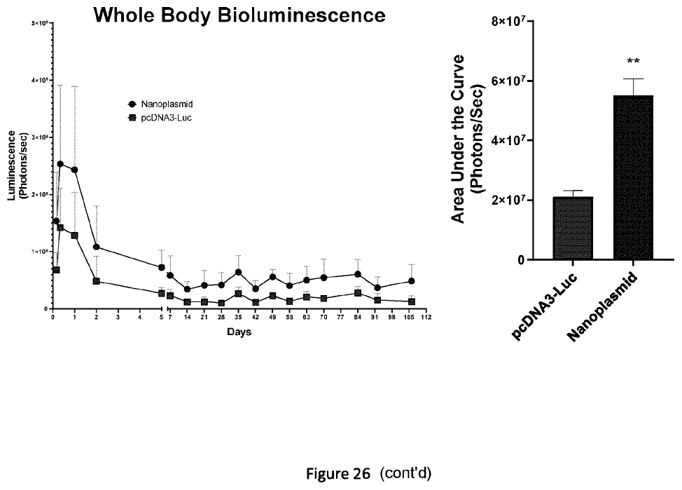

The presence of anti-FAST antibodies was also assessed in these animals at 25 days post-administration of 6 mg/kg pDNA FAST-PLVs. Anti-FAST antibodies were detected in one of the three monkeys at a level of 144.72±13.5 ng/mL. To determine whether the detected levels anti-FAST antibodies have neutralizing activity, the activity of pDNA-GFP PLVs in the presence of serum from repeatedly dosed and control animals was assessed, and no reduction in GFP expression was observed when serum treated FAST-PLVs were used to transfect 3T3 cells in vitro (FIG. 17a). Taken together, these data indicate that FAST-PLVs are safe and well-tolerated in non-human primates, with broad biodistribution and evidence for significant delivery to extrahepatic organs.

### Example 6: The Utility of the Current Delivery Platform was Also Demonstrated in a Gene Therapy Mouse Model for Muscle Wasting Disorders, Showing Increased Hindlimb Muscle Size and Grip Strength with FAST-PLV Follistatin Gene Therapy

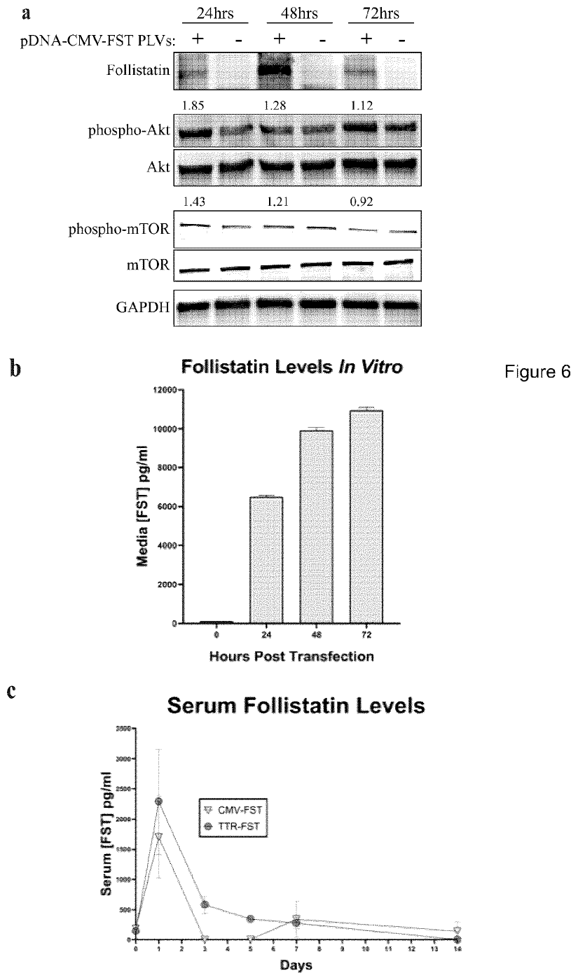

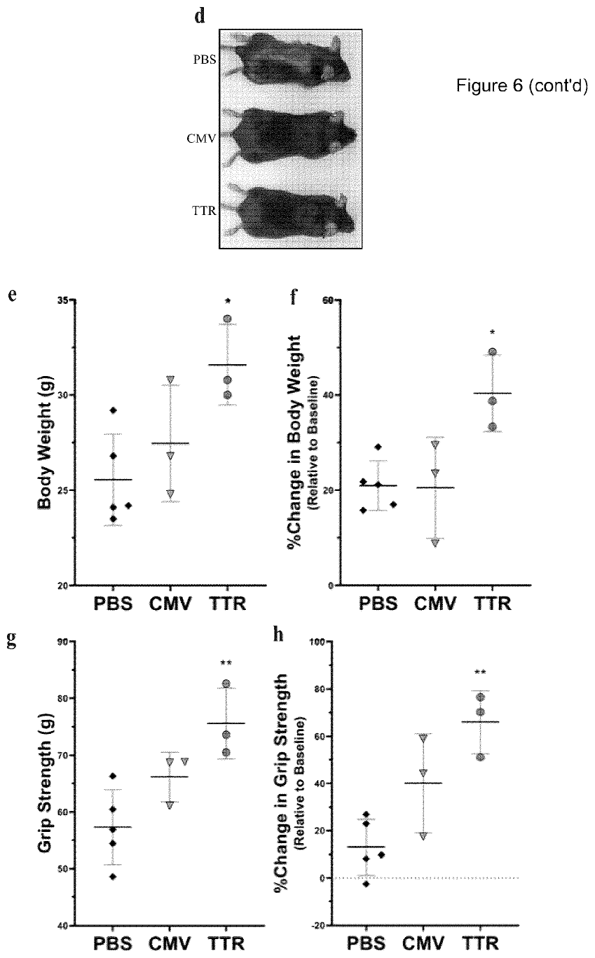

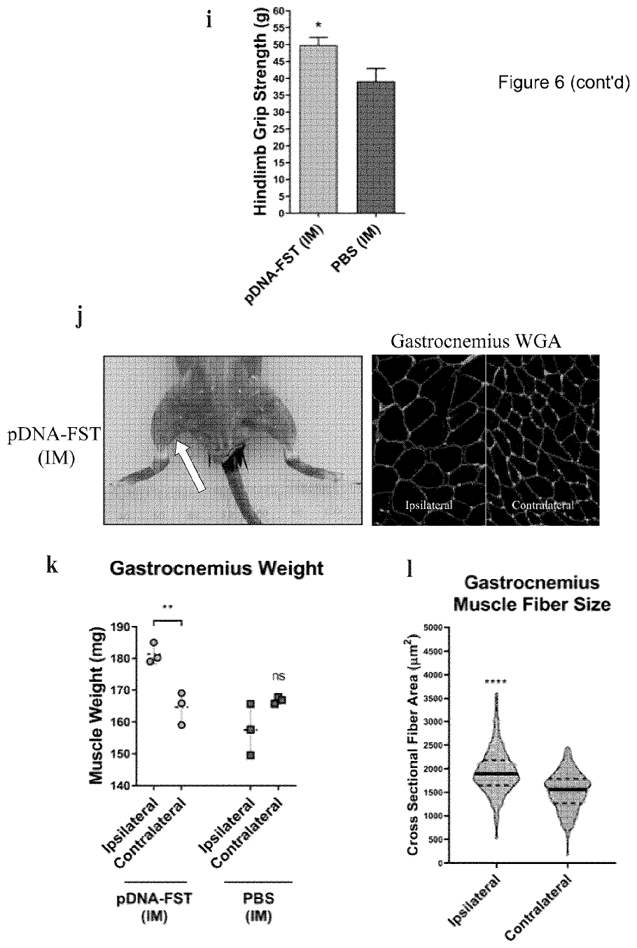

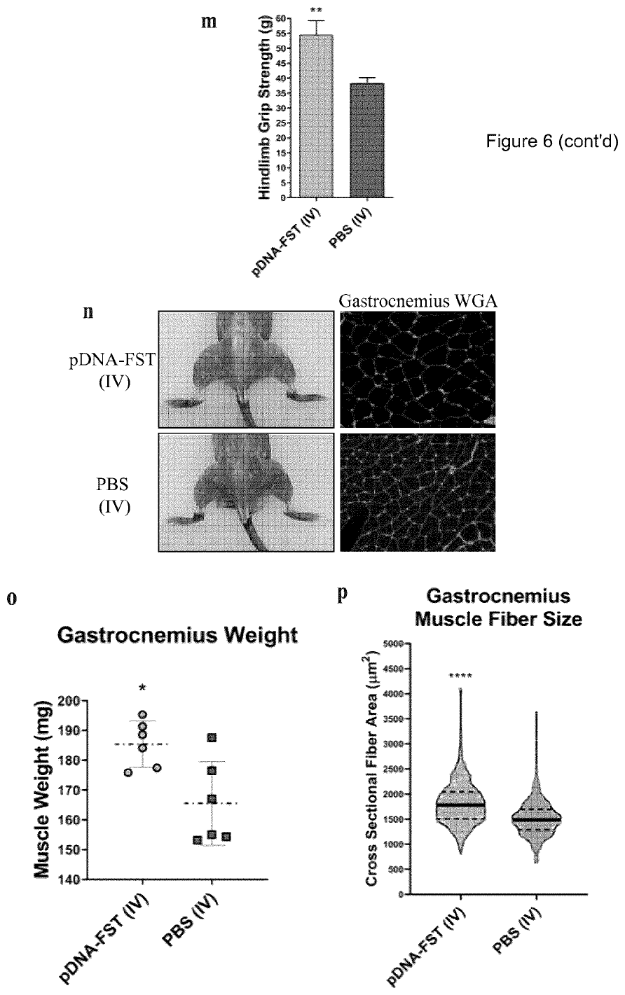

To determine the ability of FAST-PLVs to deliver pDNA encoding a therapeutic cargo, we developed a gene therapy approach to elevate expression of the protein follistatin (FST). FST facilitates hypertrophy of skeletal muscle by exhibiting an antagonistic effect on myostatin—a member of the Transforming Growth Factor-β family that inhibits muscle growth (62). AAV gene delivery vectors have been used to evaluate FST gene therapy as a potential treatment for muscle wasting disorders (63, 64). As such, experiments were undertaken to determine if delivery of pDNA encoding the FST-344 splice variant in FAST-PLV would be a viable alternative to AAV-based therapies. When pDNA-CMV-FST FAST-PLVs were incubated with C2C12 mouse myoblasts, robust FST expression relative was observed (FIG. 6a). FST expression correlated with an increase in Akt and mTOR phosphorylation 24 and 48 hours after addition of pDNA-CMV-FST FAST-PLV (FIG. 6a) (65). FST in the growth media also increased in a time-dependent manner, with levels reaching 10,000 μg/ml 72 hours after addition of pDNA-CMV-FST FAST-PLV (FIG. 6b).

Next, the ability of systemically administered pDNA FAST PLVs using two different promoters to drive FST expression in vivo was examined. As FST is primarily produced in the liver, expression when driven by the liver promoter was evaluated, transthyretin (TTR), compared to the ubiquitous CMV promoter (66). Intravenous administration of 10 mg/kg of either pDNA-CMV-FST or pDNA-TTR-FST FAST PLVs in mice resulted in a significant spike in serum FST concentration one day after injection, with levels returning to near baseline level 3-7 days following administration. Animals receiving pDNA-CMV-FST PLVs reached a one-day peak serum FST concentration of 1700 μg/ml, while animals receiving pDNA-TTR-FST FAST PLVs had a peak FST serum concentration of 2300 μg/ml (FIG. 6c). After fifteen weeks, administration with either FST FAST-PLV gene therapy resulted in animals displaying a fuller frame and musculature, while PBS injected control animals appeared thinner with protruding pelvic and vertebral bones (FIG. 6d). Mice receiving pDNA-TTR-FST FAST PLV after fifteen weeks had significantly higher body weight compared to PBS control mice (FIG. 6e, f). This observed increase in body weight was consistent with significant increases in grip strength in these animals (FIG. 6g, h).

The relative impact of local and systemic administration of the FAST-PLV FST gene therapy was also examined. Mice were administered pDNA-CMV-FST FAST-PLV locally by single intramuscular injection of 5 mg/kg in the left gastrocnemius (GAS) muscle or systemically at 10 mg/kg. Relative to mice administered PBS control, intramuscular administration into the left GAS muscle resulted in a significant increase in hind limb grip strength (FIG. 6i). Gross dissection indicated that localized FST administration and expression resulted in a significant increase in the GAS size and weight relative to the contralateral non-injected GAS (FIG. 6j, k), and a significant increase in the cross-sectional muscle fiber area of the injected GAS relative to the non-injected GAS as shown by WGA staining (FIG. 6j, l). After systemic intravenous administration of 10 mg/kg pDNA-CMV-FST, a significant increase in hind limb grip strength was measured (FIG. 6m). Gross dissection revealed a significant increase in hindlimb muscle size and GAS weight (FIG. 6n, o). Relative to control animals, WGA staining of GAS demonstrated a significant increase in cross-sectional muscle fiber area (FIG. 6n, p). These data demonstrate that FAST-PLVs, administered locally or systemically, can effectively deliver a therapeutic pDNA payload to generate localized or body-wide effects.

Example 7: Development of a targeted FAST-PLV formulation by fusing the bombesin ligand to the carboxy-terminus of the p14endop15 chimeric FAST protein. Testing the accurate targeting of FAST-bombesin PLVs by delivering the small drug molecule cabazitaxel to prostate cancer cells in vitro and tumours in vivo.

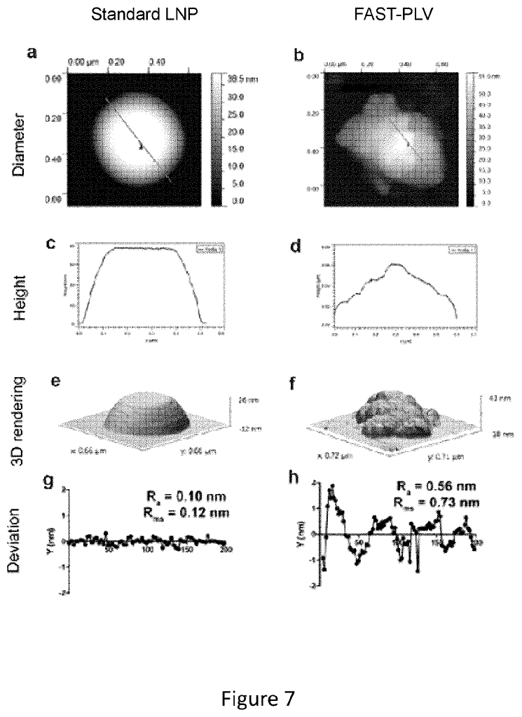

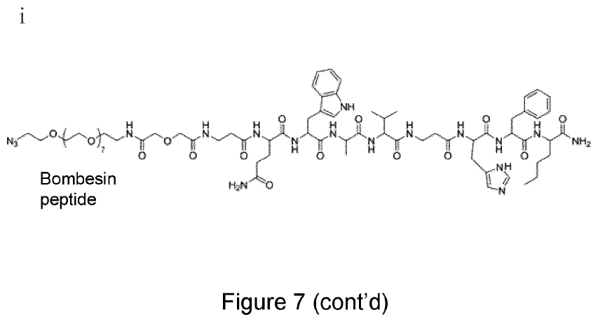

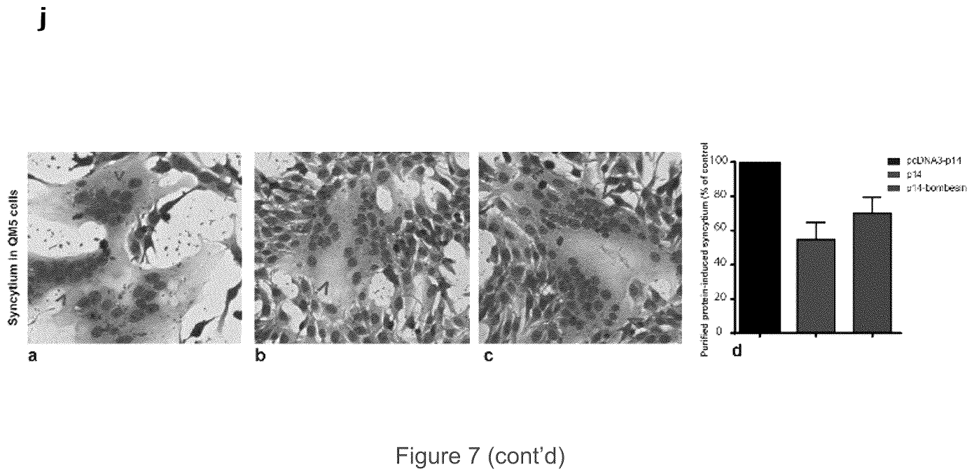

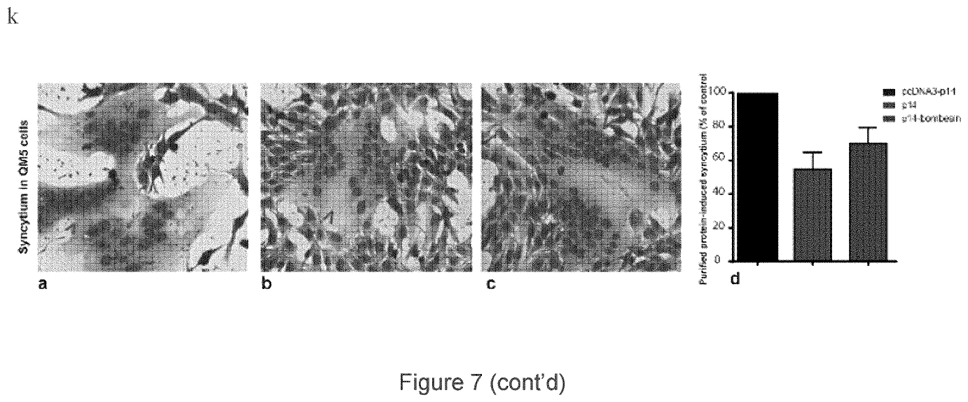

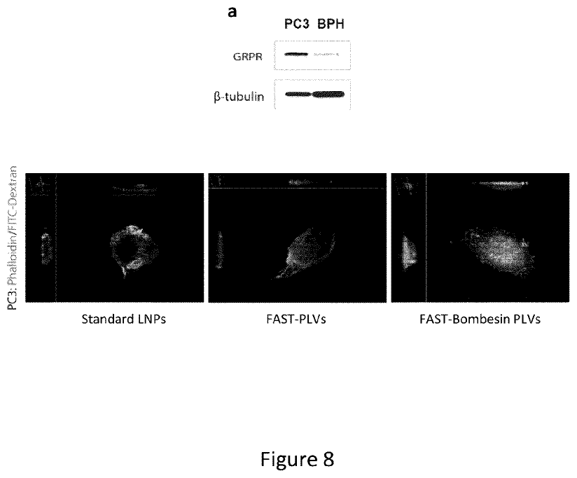

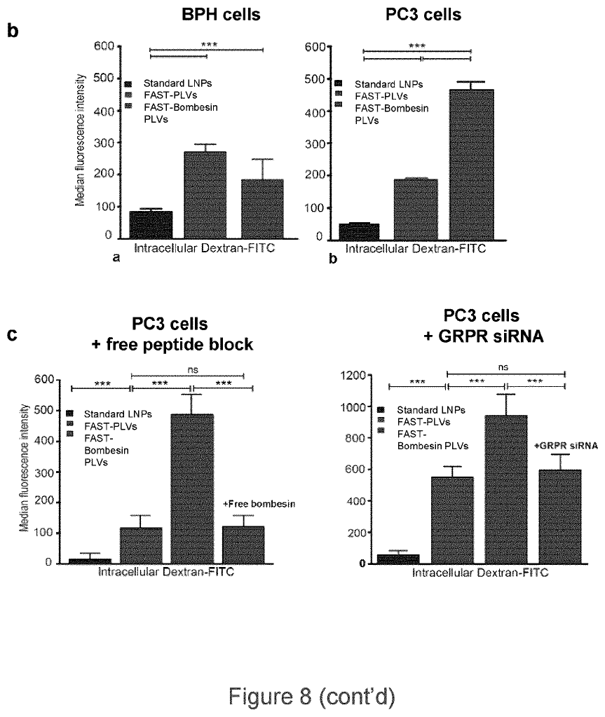

While FAST-PLVs utilize passive targeting via the enhance permeability and retention (EPR) effect to preferentially accumulate in tumours, modified FAST proteins were synthesized that incorporate additional targeting moieties to increase their selectivity for cancer cells. (67-74) An in-frame bombesin peptide to the C-terminus of the FAST protein was added and characterized the particles by AFM compared to LNPs (FIG. 7a-h, k). Bombesin is a 14-amino acid peptide ligand that binds to the Gastrin Releasing Peptide receptor (GRPR) that has been validated for PET-imaging of prostate cancer (FIG. 7i) (75-78). FAST-bombesin proteins retain fusion activity in syncytia assays (FIG. 7j). GRPR expression is increased in the majority of prostate cancer cells versus normal prostate tissue and is detected in up to 57% of bone metastasis. (79). Bombesin ligand directed targeting of FAST-PLV delivery of therapeutic cargo was characterized by analysis of the delivery of fluorescently labeled dextran to high GRPR-expressing prostate cancer cells, PC3. The low expressing GRPR cell line BPH was used as a comparison (FIG. 8a, b). Adding free bombesin or GRPG siRNA reduced delivery of FITC-Dextran suggesting that the FAST-Bombesin chimera was able to target the cell (FIG. 8c).

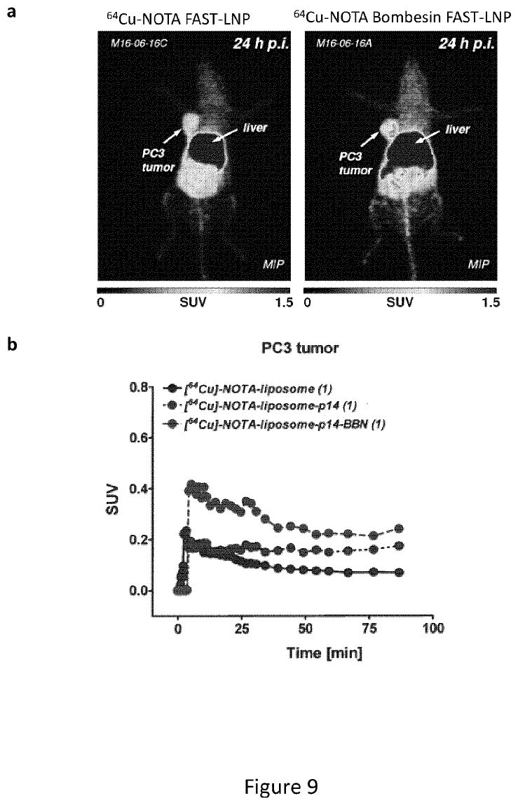

In vivo studies showed that selective targeting of prostate cancer tumors in vivo by FAST-bombesin is facilitated by GRSR receptors. Positron emission tomography (PET) imaging of PC-3 tumor uptake of 64Cu-labeled FAST-PLV formulations, (FIG. 9a, left panel) without fused bombesin and (right panel) with fused bombesin. (b), The standardized uptake value (SUV) showing the signal decay over 90 minutes (FIG. 9a, b).

The emergence of chemotherapy as a survival-improving treatment for cancer has focused attention on the need for effective prevention and management of side effects. For prostate cancer, the most recent chemotherapeutic agent in this setting is cabazitaxel, approved for use when the disease progresses on or after docetaxel, another taxane chemotherapy. (80, 81) Experience with cabazitaxel shows that its side effects, notably neutropenic complications, diarrhea and fatigue/asthenia occur more frequently and severely compared with docetaxel, which has significantly limited its use in this vulnerable patient population. (82-85)

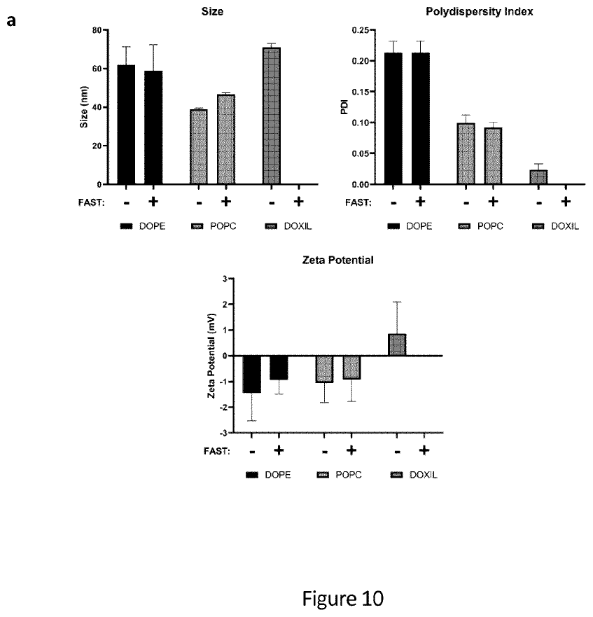

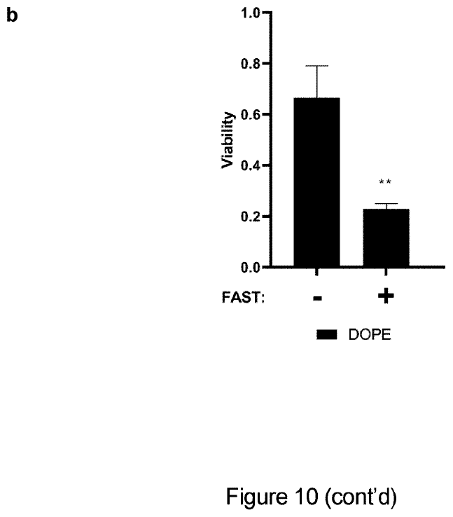

To address this, a formulation of cabazitaxel with improved efficacy and safety characteristics by encapsulating it in PLVs formulated with bombesin ligand-targeted fusion associated small transmembrane (FAST) proteins was developed. When formulated in PLVs, FAST proteins catalyze the direct mixing of lipids between the PLV and the plasma membrane of target cells that display the target receptor. The ability of FAST-PLVs decorated with bombesin peptides to specifically target cancer cells that express high levels of gastrin-releasing peptide receptors (GHSR) while avoiding normal healthy cells was assessed. The experiments were directed to prostate cancer but also evaluated activity on other cancers such as breast and pancreatic cancer that also over-express GHSRs. (86) A novel FAST protein fused with the 14 residue bombesin peptide that targets GHSR was expressed and purified, and a FAST-PLV formulation that incorporates this protein was optimized. The FAST-bombesin PLVs encapsulating cabazitaxel was characterized with respect to ligand display, size, polydispersity and surface characteristics (FIG. 10a). Incorporation of FAST protein into the DOPE based lipid formulation encapsulating cabazitaxel significantly enhanced the chemotoxicity.

### Example 8: Targeting C14orf142 Using siRNA FAST-PLV Therapy Will Reduce ccRCC Tumor Growth and Metastasis In Vivo

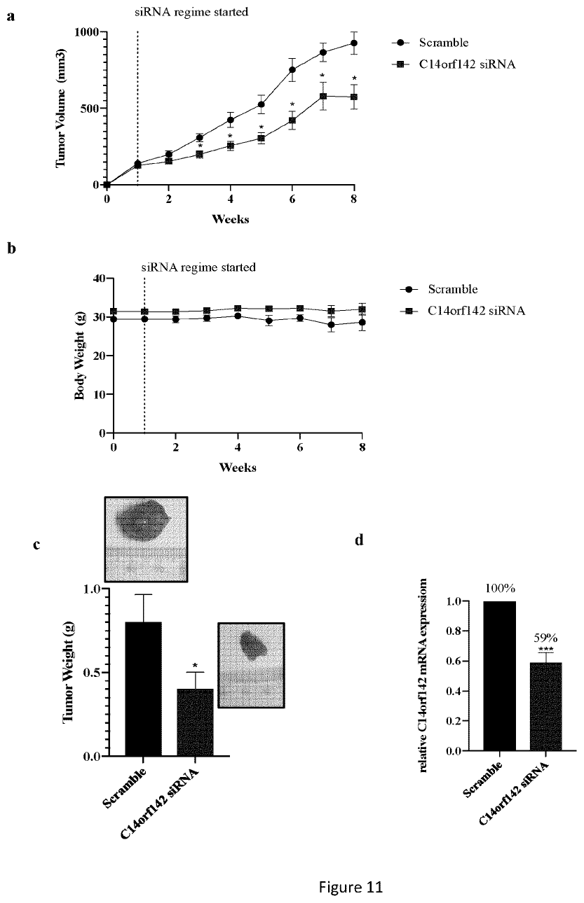

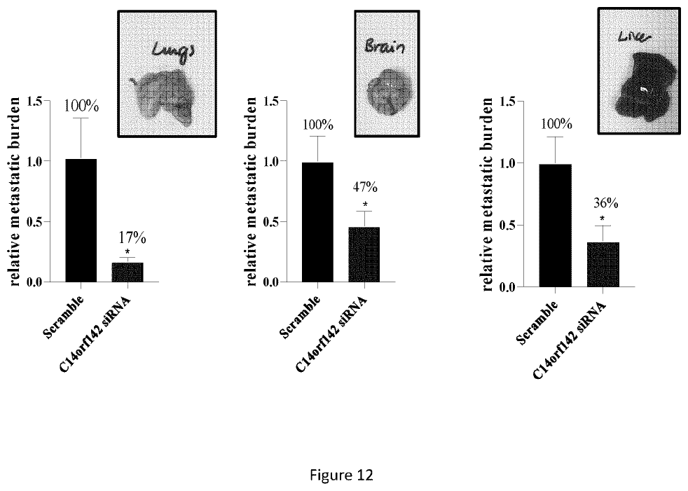

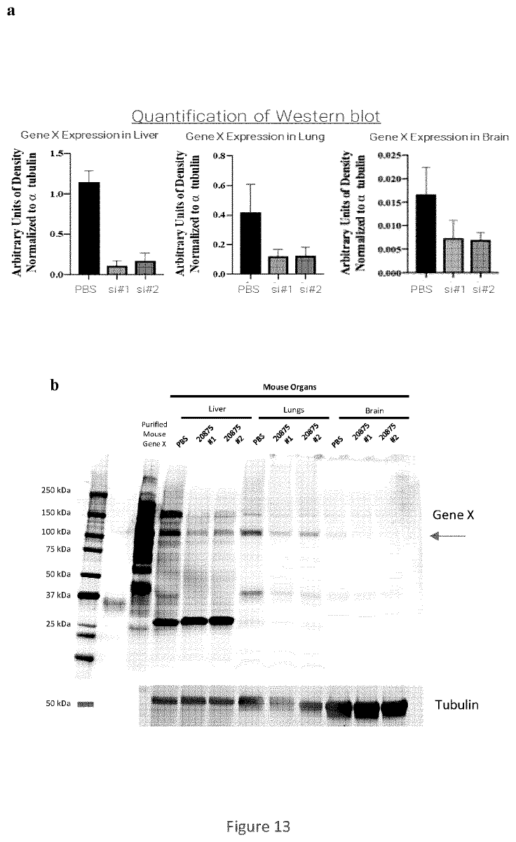

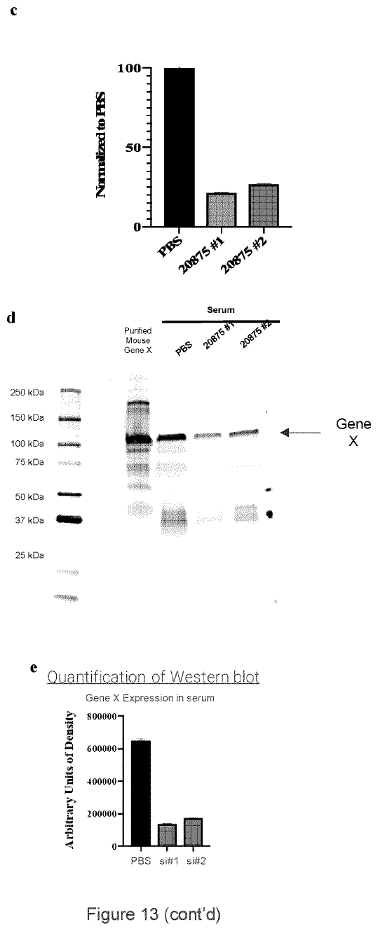

A panel of novel functional genes required for productive cell motility and successful metastatic dissemination in vivo was determined. One of the novel protein targets on the panel encodes for nuclear protein C14orf142, which is up regulated in metastatic clear cell RCC (cRCC). C14orf142, recently identified at GON7, is a component of the EKC/KEOPS complex required for the formation of a threonylcarbamoyl group on adenosine at position 37 in tRNAs that read codons beginning with adenine. Without being bound by theory, GON7 likely supports the catalytic subunit in the complex. To characterize the impact of C14orf142 on clear cell RCC vascular extravasation and distant metastasis in vivo eight-week-old male immunodeficient (NSG) mice were injected subcutaneously in the right flank with hypertriploid renal cell carcinoma (RCC) cells (7 million 786-O RCC cells). Seven days post-injection, first round of the negative control payload (Scramble) vs C14orf142 siRNA treatment was started. The dose was 150 μg twice a week via tail vein injection and the study endpoint was eight weeks (once control mice tumors reached 1 cm3 tumor volume). Administration of C14orf132 siRNA-FAST PLVs via intravenous route of administration reduced ccRCC tumour growth and metastasis (FIGS. 11, 12). In vivo evaluation of siRNA (20875) delivery to the liver by FAST-PLV (FIG. 13).

### Example 9. A Large Library of Chimeric FAST Proteins were Created

The FAST protein family comprises six structurally similar members named according to their molecular mass in Daltons (p10, p13, p14, p15, p16, and p22), but with no conserved sequence identity. All six known FAST proteins show a bipartite membrane topology with the single transmembrane domain connecting a minimal N-terminal ectodomain of ˜19-40 residues to a longer C-terminal cytoplasmic endodomain (46).

Structural motifs contained within these domains and the rate and extent of syncytium formation vary considerably between family members (47). Along with specific TMD domain features (40, 52), these motifs function in conjunction to remodel membranes and promote membrane fusion. The fusion activity of FAST protein family members as measured by syncytia assay ranges from high (p14) to medium high (p15) to low (p10).

FAST protein ectodomains typically have a myristate moiety on the penultimate N-terminal glycine and all contain diverse membrane-destabilizing fusion peptide motifs. For example, p14 contains a proline-hinged loop and p15 contains a type II polyproline helix (48, 49). The essential myristoylation (myr) motif (GxxxS/T) functions in conjunction with the adjacent 14-residue conserved region (NFVNHaPgEAlvtGLeK) as a fusion peptide (FP) (residues 2-21) promoting rapid lipid bilayer destabilization and membrane merger.

The C-terminal endodomain comprise three functional elements, an intrinsically disordered cytoplasmic tail (87, 88), a juxtamembrane polybasic motif (89), and an amphipathic α-helix (hydrophobic patch) (87, 90). The endodomain interacts with cellular partners (e.g., PPPY motif in p14 binds actin remodellers) needed to promote cell-cell membrane fusion and syncytium formation.

FAST proteins are modular because different elements or domains can perform redundant functions; the p15 proline-rich region, the palmitoylated cysteine residues of p10 and the hydrophobic patch present in all FAST proteins are diverse domains that destabilize membranes.

The modular nature of FAST proteins was exploited to determine whether a specific combination of motifs could be assembled into a recombinant FAST protein with enhanced fusion activity (47). The inventors focused on all transmembrane, endodomain and ectodomain combinations of p10, p13, p14, p15, p16, and p22.

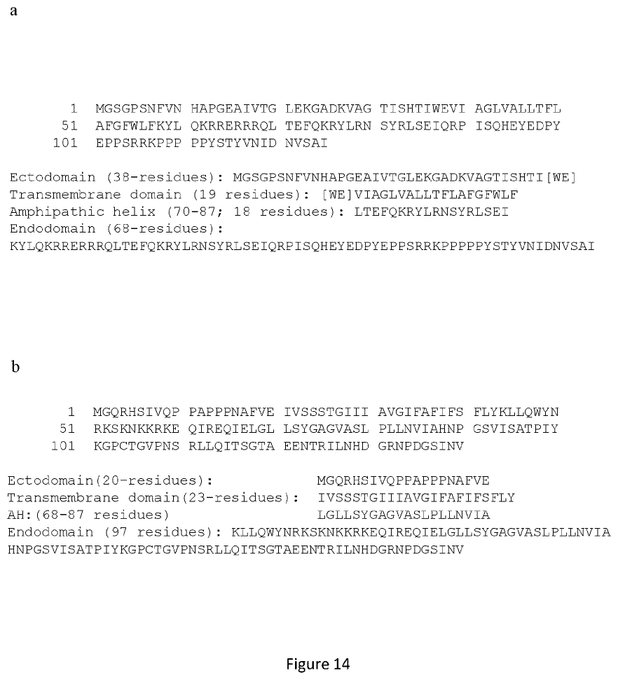

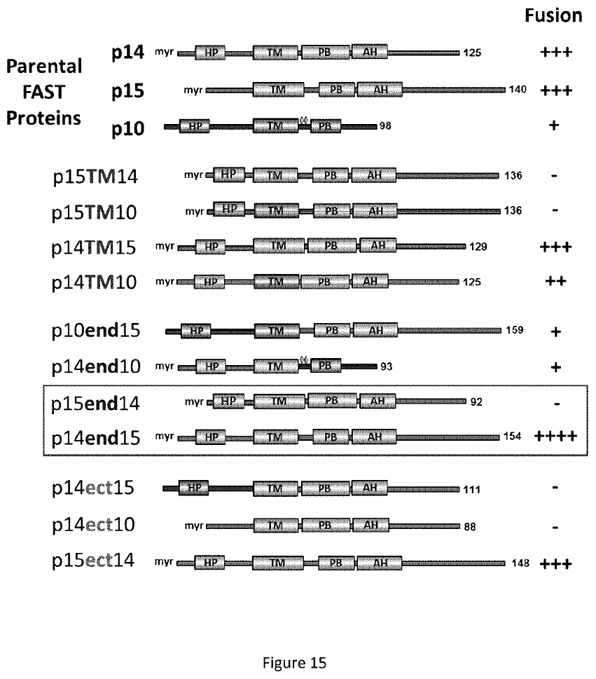

The p14 clone was also used as a template for sequential PCR using nested primers to create pl4ectol0 and p14endol0, in which the ecto- or endodomain of p14 was replaced by that of ARV p10, respectively. The p15 clone was used as a template for sequential PCR with nested primers to created pi4endo 15 in which the p14 endodomain was replaced by that of p15. In each case, the protein that contributes two of the three domains is referred to as the “backbone”. The p14 protein is the only member of the FAST protein family for which a complete chimeric library has been created in which all domains have been individually replaced by those of ARV p10 or p15 (FIG. 14). To engineer a novel FAST protein hybrid with enhanced fusion activity, a chimeric p14 and p15 FAST protein library where the ectodomain, TMD, or endodomain of p14 were substituted with the corresponding domain of p15, and the fusion activity of each construct was ranked by activity measured in a syncytia formation assay (FIG. 15).

### METHODS

### Materials

The following lipids were purchased from NOF Co. (Tokyo, Japan): 1,2-di-O-octadecenyl-3-trimethylammonium propane (DOTMA), 1,2-dioleoyl-3-dimethylammonium-propane (DODAP), 1,2-Dioleyloxy-3-dimethylaminopropane (DODMA), 1,2-dimyristoyl-sn-glycero-3-methoxypolyethylene glycol-2000 (DMG-PEG). 2-dioleoyl-sn-glycero-3-phosphoethanolamine (DOPE) and 1,2-dioleoyl-3-trimethylammonium-propane (DOTAP) were purchased from Avanti Polar Lipids (Alabaster, United States). DLin-MC3-DMA (MC3) was purchased from Precision Bio Laboratories (Edmonton, Canada). Plasmid DNA (pDNA) with the cytomegalovirus (CMV) promoter driving the DNA-encoded inserts, green fluorescent protein (GFP), and firefly luciferase (FLuc) was cloned into the p10 plasmid vector produced by Entos Pharmaceuticals (Edmonton, Canada). These pDNA preps were expanded and purified by Precision Bio Laboratories. pDNA encoding the follistatin 344 splice variant under the transthyretin (TTR) and CMV promoters was cloned into the NTC Nanoplasmid and produced by Nature Technology Company (Lincoln, United States). CleanCap mRNA with mRNA-encoded inserts; monomeric red fluorescent protein (mCherry), enhanced green fluorescent protein (eGFP), and firefly luciferase (FLuc) was purchased from TriLink Biotechnologies (San Diego, United States). Lipofectamine 2000 and CyQUANT LDH Cytotoxicity Assay was purchased from Thermo Fisher Scientific (Edmonton, Canada). Harris modified hematoxylin was purchased from Fisher Scientific (Ottawa, Canada). Eosin Y and resazurin were purchased from Millipore Sigma (Oakville, Canada). Anti-Firefly Luciferase antibody (ab181640) and anti-GAPDH antibody (ab8245) were purchased from Abcam (Cambridge, United Kingdom). Rabbit polyclonal p14endo15 antibody was produced by New England Peptide (Gardner, United States), using the target sequence Ac-PSNFVNHAPGEAIVTGLEKGADKVAGTC-Amide. Goat anti-follistatin antibody (AF669) was purchased from R&D systems (Minneapolis, United States). Phospho-Akt Ser473 (4069), pan-Akt (2920), phosphor-mTOR Ser2448 (2971), and mTOR (2972) antibodies were purchased from Cell Signaling Technology (Danvers, United States).

### Cells and Culturing

Quail fibrosarcoma (QM5) cells were cultured in Medium 199 with 3% fetal bovine serum (FBS; Sigma) and 0.5% penicillin/streptomycin (Thermo Fisher Scientific, Edmonton, Canada). Human hepatocellular carcinoma cells (HEP3B), human non-small cell lung cancer cells (NCI-H1299), human lung fibroblast cells (IMR-90 and WI-38), mouse embryo fibroblast cells (3T3), VERO CCL-81 cells (Cercopithecus aethiops epithelial kidney cells), and mouse myoblasts (C2C12) were purchased from ATCC (Manassas, VA) and cultured in high glucose-DMEM with 10% FBS and 1% penicillin/streptomycin. Human retinal pigmented epithelium cells (ARPE-19) were a gift from Dr. Ian MacDonald (University of Alberta) and were cultured in DMEM/F12 with 10% FBS and 1% penicillin/streptomycin. Human umbilical vein endothelial cells (HUVEC) were a gift from Dr. Allan Murray (University of Alberta) and were cultured in EGM-2 BulletKit (Lonza, Cat No. CC-3162). Sprague-Dawley Rat Primary Hepatocytes were purchased from Cell Biologics and were cultured in Complete Hepatocyte Medium Kit from Cell Biologics (Cat No. M1365). Primary rat astrocytes were purchased from Cell Applications Inc. (San Diego, United States) and cultured in rat astrocyte growth medium (Cat No. R821-500). Mammalian adherent cells were grown in tissue-culture treated 75 cm² flasks (VWR 10062-860) until cells were 80% confluent or nutrients in the media are depleted in a 37° C. incubator with humidified atmosphere of 5% CO₂ (Nuaire NU-5510). Spodoptera frugiperda pupal ovarian tissue (Sf9) cells were stepwise cultured at 25 C to 2×10⁶-4×10⁶ cells/mL from 25 mL to 100 mL and finally into a 2 L wave bioreactor. The Trypan Blue assay was used to check for cell viability.

### Purification of FAST Proteins

The Sf9 cells were lysed, and supernatant was clarified by 0.2 μm filtration. The FAST proteins were purified from the supernatant using an AKTA affinity purification column, followed by dialysis and cationic exchange purification (AKTA). Protein samples were quality control analyzed by SDS-PAGE and Western blot; functional validation was done via syncytia formation assay.

### Western Blot

Cells were lysed in ice-cold Pierce RIPA buffer (Thermo Scientific, Cat. No. 89900). Protein amount was determined using Pierce BCA protein assay (Thermo Scientific, Cat. No. 23225). Equal amounts of total protein from each lysate were loaded onto Mini-PROTEAN 4-20% Gradient TGX precast gels (BIO-RAD, Cat. No. 456-1095). Separated protein was transferred to nitrocellulose membranes (BIO-RAD, Cat. No. 1620112). Membranes were blocked with fluorescent western blocking buffer (Rockland, Cat. No. MB-070) for 1 hour at room temperature. Primary antibodies were diluted 1:1000 in blocking buffer and added to the membranes overnight at 4° C. with shaking. Goat anti-rabbit Alexa Fluor 680 (Thermo Scientific, Cat. No. A27042), donkey anti-goat Alexa Fluor 680 (Thermo Scientific, Cat. No. A-21084), or goat anti-mouse Alexa Fluor 750 (Thermo Scientific, Cat. No. A-21084) were diluted 1:10000 in blocking buffer and added for 1 hour at room temperature in the dark. Membranes were visualized on the LI-COR Odyssey.

### Syncytia Formation and Inhibition

QM5 quail fibrosarcoma cells were seeded at a density of 3.5×10⁵ in twelve well cluster plates in Medium 199 containing 10% FBS and cultured overnight before transfecting with Lipofectamine 2000 and 1 μg of pcDNA3 plasmid expressing either p14, p14endo15, or p15 per manufacturer's instructions. Cells were fixed in 3.7% formaldehyde in HBSS at the indicated intervals post-transfection and stained with Hoechst 33342 and WGA-Alexa 647 per manufacturer's instructions. Images (n=5) of each condition were captured on a Zeiss Axio Observer A1 inverted microscope at predetermined coordinates within the well (n=3). Syncytia were then manually identified with syncytial and total nuclei quantified using FIJI imaging software.

### Lipid Formulations

The lipid formulations designated as 28M, 33T, 37N and 41N were made by combining the cationic lipid (DOTAP), ionizable lipid (DODAP and/or DODMA), helper lipid (DOPE) and PEGylated lipid (DMG-PEG2000) in the following lipid molar ratios: 28M (24:42:30:4), 33T (24:21:21:30:4), 37N (6:60:30:4) and 41N (0:66:30:4). The lipids were heated in a 37° C. water bath for 1 min, vortexed for 10 seconds each, then combined and vortexed for 10 seconds. The combined lipid mixture was dehydrated in a rotavapor at 60 rpm for 2 hours, under vacuum, then rehydrated with 14 mL 100% ethanol, and sonicated (Branson 2510 Sonicator) at 37° C., set to sonication of 60. The lipid formulation was aliquoted in 500 μL batches and stored at −20° C. MC3-LNP formulation was composed of DLin-MC3-DMA/DSPC/Cholesterol/PEG-lipid with the molar ratio 50:10:38.5:1.5 (11).

### Nucleic Acid Quantification

Nucleic acid concentration and purity was measured via absorbance at 260 nm and 280 nm using the Nanodrop method according to the manufacturer's instructions (Nanodrop 2000 Spectrophotometer, Thermo Scientific, Edmonton, Canada).

### FAST-PLV Construction

The FAST-PLVs were made with lipid formulation 41N unless otherwise stated. The NanoAssemblr Benchtop microfluidics mixing instrument (Precision NanoSystems, Vancouver, BC, NIT0013, and NA-1.5-88, respectively) was used to mix the organic and aqueous solutions and make the PLVs. The organic solution consisted of lipid formulation. The aqueous solution consisted of nucleic acid cargo, 5 nM FAST (p14endo15) protein, and 10 mM acetate buffer (pH 4.0). The Benchtop NanoAssemblr running protocol consisted of a total flow rate of 12 mL/min and a 3:1 aqueous to organic flow rate ratio. PLVs were dialyzed in 8000 MWCO dialysis tubing (BioDesign, D102) clipped at one end. The loaded tubing was rinsed with 5 mL of double distilled water and dialyzed in 500 mL of Dialysis Buffer (ENT1844) with gentle stirring (60 rpm) at ambient temperature for 1 hour and was repeated twice with fresh Dialysis Buffer. PLVs were concentrated using a 100 kDa Ultra filter (Amicon, UFC810096) according to the manufacturer's instructions. PLVs were filter sterilized through 0.2 μm Acrodisc Supor filters (Amicon, UFC910008)

### In Vitro Transfection

Cells were counted using a hemocytometer, and 3,000-5,000 cells were seeded to 96-well or 20,000-40,000 cells to 48-well tissue-culture treated plates and left overnight. The cells were transfected with 10-2000 ng of pDNA encapsulated in FAST-PLVs, MC3-LNPs, or Lipofectamine 2000 for 96-well plate (300 μl cell culture media final) and 1000 ng for 48-well plates (1000 μl cell culture media final). Lipofectamine 2000 was prepared according to manufacture instructions. The optimal transfection time for mRNA is 24-48 hours and 72-96 hours for pDNA. A luciferase reporter assay was used to measure expression levels of FLuc in different cell lines. Cell culture media was removed from cells growing in a 96-well plate, and cells washed with 1× PBS. A 50-microliter aliquot of reporter lysis buffer (Promega E397A) was added to the cells. The cells were mixed and incubated at room temperature for 10-20 mins. D-luciferin (150 μg/mL, GOLDBIO, LUCK-100) was dissolved in 100 mM Tris-HCl (pH 7.8), 5 mM MgCl₂, 2 mM EDTA, 4 mM DTT, 250 μM acetyl-CoA, and 150 μM ATP (129). The luciferin substrate (100 μL) of was added to each well immediately before measurement. Luminescence was measured via the FLUOSTAR Omega fluorometer using the MARS data analysis software for analysis. Green fluorescent protein (GFP) or mCherry expressing cells were processed for flow cytometry analysis. The cells were trypsinized and resuspended in 400 μL (per well of 48 well plate) of FACS buffer, then transferred to a 5 mL flow cytometry tube (SARSTEDT 75×12 mm PS Cat. no. 55.1579) and analyzed with a BD LSRFortessa X20 SORP. Mean fluorescence intensity (MFI) presented on the Fluorophore⁺ population unless otherwise stated.

### PLVs Characteristics and Encapsulation Efficiency

PLVs made by NanoAssemblr Benchtop were diluted 1:50 to 1:20,000, depending on concentration, with twice 0.2 μm syringe-filtered PBS buffer. Particle size, polydispersity index (PDI), and zeta potential was measured on final samples using the Malvern Zetasizer Range and a Universal ‘Dip’ Cell Kit (Malvern, ZEN1002) following the manufacturer's instructions. The nucleic acid encapsulation efficiency was calculated using a modified Quant-IT PicoGreen dsDNA assay with the following modifications to the assay protocol (Thermo Fisher Scientific, Edmonton, Canada). PLVs were mixed 1:1 with TE+Triton (2%) to obtain the Total DNA Concentration, or with TE alone to obtain the Unencapsulated DNA Concentration. The DNA standards were also diluted in TE+Triton (2%), and samples were and incubated at 37° C. for 10 min, then diluted a final time with TE+Triton (1%) or TE alone, plated in a black 96 well flat-bottomed plate, and measured with a FLUOstar Omega plate reader (BMG Labtech, 415-1147). Encapsulation efficiency was calculated by using the following equation:

$$Encapsulation Efficiency = \frac{Total DNA Concentration - Unencapsulated DNA Concentration}{Total DNA Concentration} \times 100$$

### Atomic Force Microscopy

Final FAST-PLVs encapsulating pDNA were evaluated by Atomic Force Microscopy (AFM, Bruker Dimension Edge) for visual validation of the measured particle size. The PLVs were diluted with 0.1 μm filtered PBS to 0.1 μg/mL. An aliquot (2 μL) of the diluted sample solutions was immediately spread on a clean glass slide. The sample was dried at ambient temperature (25° C.) for 5 min and any excess aqueous solution was removed with filter paper. The sample was dried for another 15 minutes before imaging at a scan speed of 1 Hz. Tapping mode was carried out using a Ted Pella Tap300 cantilever with a quoted spring constant of 20-75 N/m. 2 D and 3 D images of different zones were examined due to the limitation of small, scanned areas by AFM. Height, Phase and Amplitude mode was used for image analysis using Gwyddion software.

### Transmission Electron Microscopy

Final FAST-PLVs encapsulating pDNA were evaluated by transmission electron microscopy. 5 μL aliquots of thousand-fold diluted FAST-PLVs, were placed on 300 mesh carbon-coated copper grids for an hour to dry on the surface, followed by two washes with 0.1 μm filtered water. After removal of excess liquid, samples were negatively stained using 0.1 μm filtered 1% uranyl acetate. The dried samples were examined in a JEOL JEM-ARM2000F S/TEM electron microscope.

Viability Assay with Alamar Blue

Cell cultures in a 96 well plates were treated with test compounds at indicated concentrations. After 24-96 hours, a 1/10 volume of Alamar blue solution (440 μM Resazurin; Sigma R7017 5GM) was added to the cells in culture medium and incubated for 2-4 hours at 37° C. 5% CO₂. The Omega Fluostar (BMG LabTech) plate reader was used to measure the fluorescence (excitation wavelength of 540 nm and emission at 590 nm) of the treated cells. Cell viability was calculated using the following formula:

$$Viability = \frac{Treated Absorbance - Media Background Absorbance}{Vehicle Absorbance - Media Background Absorbance}$$

### Lactate Dehydrogenase (LDH) Cytotoxicity Assay

VERO cells were seeded into 96 well plates, and the CyQUANT LDH Cytotoxicity Assay was conducted to determine the toxicity of different lipid formulations following manufacturers' instructions. In brief, pDNA-FLuc was encapsulated within each lipid formulation (28M, 33T, 37N, and 41N) and added to cells at a pDNA concentration of 1.5 nM. Twenty-four hours after pDNA addition, 50 μL of cell culture media was collected for LDH absorbance. Cytotoxicity was calculated using the following equation:

$$\% Cytotoxicity = \frac{Lipid Treated LDH Activity - Vehicle LDH Activity}{Maximum LDH Activity - Vehicle LDH Activity} \times 100$$

### Rodent Experiments: Ethics and Study Design

All animal studies were carried out according to the guidelines of the Canadian Council on Animal Care (CCAC) and approved by the University of Alberta Animal Care and Use Committee. In vivo studies were done using 25 to 35 g body weight, male and female C57BL/6 (Charles River Laboratories, Saint-Constant, Canada). Animals were group-housed in IVCs under SPF conditions, with constant temperature and humidity with lighting on a fixed light/dark cycle (12-hours/12-hours). Intravenous injection occurred via the lateral tail vein with 200 μL of the test agent. Intramuscular injection occurred in the semitendinosus and semimembranosus muscle of the hind limb with 50 μL of the test agent. Blood was collected via the lateral tail vein or cardiac puncture at indicated time points into serum collection tubes (Sarstedt, Montreal, Canada). Hindlimb grip strength was measured in quintuplicate using a T-bar attachment on the BIOSEB grip strength meter (Panlab, Cat. No. BSBIOGS3BS 76-1066). Bone density was calculated using the in vivo imaging system (In Vivo Xtreme, Bruker, Montreal, Canada) (130).

### Whole Body and Ex Vivo Bioluminescence

At indicated time points after the injection of the FAST-PLVs, mice were injected intraperitoneally with 0.25 mL D-luciferin (30 mg/mL in PBS) and allowed to recover for 5 minutes. The mice were then anesthetized in a ventilated anesthesia chamber with 2% isoflurane in oxygen and imaged ˜10 min after D-luciferin injection with an in vivo imaging system (In Vivo Xtreme, Bruker, Montreal, Canada). All images are taken with a non-injected control mouse to serve as a reference point to determine the lower threshold of each image. Grouped experiments are presented with the upper threshold being held consistent between time points; however, the lower threshold is set based on the signal of the non-injected control mouse at the point when it no longer shows any signal. Quantification of the luminescent signal was done using Bruker Molecular Imaging Software. A manual ROI was drawn to encompass the entire area of each mouse. The sum intensity (photons/second) from the non-injected control mouse was subtracted from the sum intensity from each experimental mouse to normalize different timepoints and control for background signal drift on each image. For ex vivo images, major organs were paired with those from a non-injected control mouse. 30 mg/ml D-Luciferin was mixed at a 1:1 ratio with the in vitro 150 μg/mL D-Luciferin described above (129) and added to the organs immediately before imaging. Non-injected control mouse organs were included with each set as a reference point to determine the lower threshold.

### Non-Human Primate Studies

All in-life NHP procedures were carried out by Virscio, Inc, under the guidance of the Institutional Animal Care and Use Committee (IACUC) of the St. Kitts Biomedical Research Foundation (SKBRF), St Kitts, West Indies. SKBRF research facility is fully accredited by the Association for Assessment and Accreditation of Laboratory Animal Care International (AAALAC International). African green monkeys (Chlorocebus sabaeus) are an invasive species on the island of St. Kitts and were procured locally using approved practices with IACUC oversight. Animals were housed in well-ventilated outdoor enclosures for the duration of the study. PLVs were infused into the saphenous or cephalic vein at a rate of 2 mL/min. Blood was collected via femoral or saphenous vein phlebotomy following overnight fasting under ketamine/xylazine anesthesia. Blood was transferred to Vacutainer serum collection tubes without clot activators (BD Medical, New Jersey, United States) for 1 hour at room temperature to allow clotting followed by centrifugation at 3000 rpm for 10 minutes at 4° C. At scheduled sacrifice, animals were sedated with ketamine and xylazine (8 mg/kg and 1.6 mg/kg respectively, IM) and euthanized with sodium pentobarbital (25-30 mg/kg IV). Upon loss of corneal reflex, transcardial perfusion was performed with chilled, heparinized 0.9% saline, and the brain and spinal cord were removed. Following perfusion, a gross necropsy was conducted. All abnormal findings were recorded, and associated tissues were collected and post-fixed in formalin for histopathology. Serum samples were sent to Antech Diagnostics (Los Angeles, CA) for clinical chemistry evaluation.

Biodistribution of pDNA in Excised Tissues

Adult green monkeys (Chlorocebus sabaeus) were intravenously infused with 1 mg/kg pDNA encapsulated within FAST-PLVs and sacrificed two days later. DNA was isolated using the DNeasy Blood & Tissue Kit (Qiagen, Toronto, Canada) protocol following the manufacturer's instructions. Cells were centrifuged for 5 min at 300×g, the pellet resuspended in 200 μL PBS, and 20 μL proteinase K was added plus 200 μL Buffer AL (without added ethanol). The mixture was vortexed and incubated at 56° C. in a Thermomixer (Labnet International, Inc, Edison, NJ) for 10 min. The levels of pDNA in excised tissues were measured using a PCR assay with primers specific to the pDNA backbone. A standard curve was generated using known amounts of pDNA and used to quantify the amount present in each tissue.

### Meso Scale Discovery

The Mesoscale Discovery QuickPlex SQ 120 (MSD, Rockville, MD) was used with mouse and non-human primate samples as per the manufacturer's instructions. The data was analyzed with MSD Workbench 4.0 software, following the software protocol. The Meso Scale Discovery V-PLEX NHP cytokine 24-Plex Kit (MSD, Rockville, MD) was used to quantitatively determine serum concentrations of 24 proinflammatory cytokines, including IFN-γ, IL-1β, IL-5, IL-6, IL-7, IL-8, IL-10, IL12/1L23 p40 Subunit, IL-15, IL-16, IL17A, CXCL1, GM-CSF, TNF-α, TNF-β, VEGF, IP10, Eotaxin, MCP-1, MCP-4, MDC, MIP-1α, MIP-1β, and TARC. The Meso Scale Discovery V-PLEX Proinflammatory Panel 1 mouse kit was used to quantitatively determine serum concentrations of 10 proinflammatory cytokines: IFN-γ, IL-1p, IL-2, IL-4, IL-5, IL-6, IL-10, IL-12p70, CXCL1 (KC/GRO), and TNF-α.

Anti-Drug Antibody Titer: Indirect Electrochemiluminescence Immunoassay (ECLIA) for p14endo15 and FLuc

Recombinant firefly luciferase protein (NBP1-48355, Novus Biologicals, Centennial, United States) or purified p14endo15 protein was coated on the standard binding plate (Meso Scale Discovery; MSD, Rockville, United States) at one μg/mL for one hour at ambient temperature with shaking. The plate was washed three times with 0.05% Tween-20 in PBS followed by the addition of Blocker A (blocking buffer, MSD). After 30 min of incubation, the plate was rewashed with PBS-T. Serially diluted p14endo15 antibody and luciferase antibody standards were prepared in Blocker A. Mouse, and nonhuman primate serum samples were diluted 1:100 in Blocker A. The antibody standards and diluted mouse serum samples were loaded to plates and incubated for one hour at ambient temperature with shaking. The plate was washed again with PBS-T followed by the addition of 1 μg/mL sulfo-tag anti-rabbit or anti-goat secondary antibody in standards (Meso Scale Discovery; MSD, Rockville, United States), and one μg/mL sulfo-tag anti-mouse secondary antibody in mouse serum samples (Meso Scale Discovery; MSD, Rockville, United States). Read buffer (Meso Scale Discovery; MSD, Rockville, United States) was added to the plate after washing with PBS-T, and the plate was read in MESO QuickPlex SQ 120 (Meso Scale Discovery; MSD, Rockville, United States).

### MicroVue Complement C3a C4d and SC5b-9 Enzyme Immunoassay

The levels of fragments of complement components such as C3a, C4d, and SC5b-9 in NHP serum were measured to determine whether PLVs activated the complement system (C3, C4 and C5). After PLVs were administered, blood was collected at 0.5, 1.0, 1.5, and 12 hours and the sera were immediately extracted. One hundred μL of serum was used to determine the levels of C3a, C4d, and SC5b-9 using QUIDEL MicroVue complement C3a/C4d/SC5b-9 Plus EIA kits (Quidel A032 XUS, San Diego, CA) according to manufacturer's instructions, including the high and low controls. A sample of serum taken before PLV administration was used to determine baseline levels of C3a, C4d, and SC5b-9. According to the manufacturer's instructions, the FLUOstar Omega microplate reader was used to measure the optical density of the samples.

### Follistatin ELISA

Serum and media follistatin levels were quantified using human follistatin ELISA kit (PeproTech, Cat. No. 900-K299) with slight modification to adapt it to the MSD system. Capture FST antibody was coated on MSD standard binding plate at one μg/ml overnight at room temperature with shaking. The plate was washed three times with 0.05% Tween-20 in PBS followed by the addition of Blocker A (blocking buffer, MSD). After 1 hour of incubation, the plate was rewashed with PBS-T. Serially diluted Follistatin standard was prepared in Blocker A with 10% mouse serum. Mouse serum samples were prepared in Blocker A with a 1:10 dilution. The serum samples and follistatin standards were incubated overnight at 4° C. with shaking. The plate was rewashed with PBS-T and biotinylated follistatin detection antibody was added at a concentration of 1 μg/ml for 2 hours at room temperature with shaking. The plate was washed three times with PBS-T followed by the addition of 1 μg/mL sulfo-tag streptavidin (Meso Scale Discovery; MSD, Rockville, United States) for 1 hour at room temperature. The plate was washed with PBS-T three times, then Read buffer (Meso Scale Discovery; MSD, Rockville, United States) was added to the plate then analyzed with the MESO QuickPlex SQ 120 (Meso Scale Discovery; MSD, Rockville, United States).

### Human Erythropoietin (EPO) ELISA

Serum EPO levels were quantified using the U-PLEX Human EPO Assay kit developed my Meso Scale Discovery (Cat. No. K151VXK-2), following manufacturer's instructions. Briefly, plates are coated with biotinylated capture antibody prior to sample and standard administration. Samples are incubated on plate for 1 hour at room temperature, following which detection antibody is added for 1 hour. MSD GOLD Read Buffer B (Meso Scale Discovery; MSD, Rockville, United States) was added to the plate and then it was analyzed with the MESO QuickPlex SQ 120 (Meso Scale Discovery; MSD, Rockville, United States).

### Histology: Immunohistochemistry and H&E Staining

Major organs, including the heart, liver, spleen, lungs, and kidneys, were collected, formalin-fixed, and paraffin-embedded. 4-6 μm sections were generated. Sections were dewaxed in xylene and rehydrated using graded ethanol to water washes. Samples for H&E staining were stained in hematoxylin for 8 minutes, briefly differentiated in acid alcohol, and blued with Scott's Tap Water (pH 8). Slides were then stained in acidified eosin for 30 seconds, and dehydrated, cleared, and then mounted. Whole slide images were generated using Panoramic SCAN (3D Histech, Budapest, Hungary) and reviewed by a certified DVM pathologist (Greenfield Pathology Services, Greenfield, United States) to evaluate the organ-specific toxicity. Heat-induced antigen retrieval for IHC samples was conducted by immersing rehydrated slides in 10 mM sodium citrate (pH 6) and heating until boiling occurred. Slides were blocked in 10% normal rabbit serum (Cat. No. 869019-M, Sigma, Oakville, Canada) with 1% bovine serum albumin (BSA, Cat. No. A9418, Sigma, Oakville, Canada) in TBS with 0.1% Tween-20 for one hour at ambient temperature. Anti-firefly luciferase antibody was diluted at 1:1000 in blocking buffer and incubated on slide overnight. Endogenous peroxidase was blocked with 3% H₂O₂ in PBS. HRP conjugated rabbit anti-goat secondary antibody (ab97100, Abcam, Cambridge, United Kingdom) was diluted to 1:200 in 1% BSA TBS with 0.1% Tween-20 and added to slides for 1 hour at ambient temperature. Samples were stained with EnVision FLEX DAB+Chromogen (GV82511-2, Agilent Dako, Santa Clara, United States) for 20 minutes. The reaction was stopped by rinsing in H₂O. Slides were counterstained with hematoxylin and dehydrated, cleared, and then mounted. Gastrocnemius utilized for determining muscle fiber area were flash-frozen in O.C.T. Compound (Fisher Scientific, Cat. No. 23-730-571) and sectioned using a cryostat. Ten μm sections were warmed to room temperature and fixed with 3.7% formaldehyde for 15 minutes. Cells were washed three times with PBS. Sections were covered with 5 μg/ml wheat germ agglutinin Alexa Fluor-488 (Thermo Scientific, Cat. No. W11261) for 10 minutes at room temperature. Cells were washed three times with PBS and mounted using ProLong Gold antifade reagent (Thermo Scientific, Cat. No. P36930). Sections were visualized using EVOS fl inverted microscope (Advanced Microscopy Group, Bothell, United States) and 7-15 images were taken per section. Cross sectional muscle fiber area was determined using MyoVision software (131).

### Statistical Analyses

A two-tailed Student's t-test or a one-way analysis of variance (ANOVA) was performed when comparing two groups or more than two groups, respectively. Statistical analysis was performed using Microsoft Excel and Prism 7.0 (GraphPad). Data are expressed as means±s.d. The difference was considered significant if P<0.05 (*P<0.05, **P<0.01, ***P<0.001, ****P<0.0001 unless otherwise indicated).

### REFERENCES

1. Roberts T C, Langer R, Wood M J A. Advances in oligonucleotide drug delivery. Nature Reviews Drug Discovery 2020
2. Thomas C E, Ehrhardt A, Kay M A. Progress and problems with the use of viral vectors for gene therapy. Nature Reviews Genetics 2003; 4:346-58
3. Ginn S L, Amaya A K, Alexander I E, Edelstein M, Abedi M R. Gene therapy clinical trials worldwide to 2017: An update. The Journal of Gene Medicine 2018; 20:e3015-e
4. Boutin S, Monteilhet V, Veron P, Leborgne C, Benveniste O, Montus M F, et al. Prevalence of serum IgG and neutralizing factors against adeno-associated virus (AAV) types 1, 2, 5, 6, 8, and 9 in the healthy population: implications for gene therapy using AAV vectors. Human gene therapy 2010; 21:704-12
5. Nayak S, Herzog R W. Progress and prospects: immune responses to viral vectors. Gene therapy 2010; 17:295-304
6. Masat E, Pavani G, Mingozzi F. Humoral immunity to AAV vectors in gene therapy: challenges and potential solutions. Discovery medicine 2013; 15:379-89
7. Ferreira V, Twisk J, Kwikkers K, Aronica E, Brisson D, Methot J, et al. Immune responses to intramuscular administration of alipogene tiparvovec (AAV1-LPL(S447X)) in a phase II clinical trial of lipoprotein lipase deficiency gene therapy. Human gene therapy 2014; 25:180-8
8. Bessis N, GarciaCozar F J, Boissier M C. Immune responses to gene therapy vectors: influence on vector function and effector mechanisms. Gene Therapy 2004; 11:S10-S7
9. Halbert C L, Rutledge E A, Allen J M, Russell D W, Miller A D. Repeat transduction in the mouse lung by using adeno-associated virus vectors with different serotypes. Journal of virology 2000; 74:1524-32
10. Priddy F H, Lewis D J M, Gelderblom H C, Hassanin H, Streatfield C, LaBranche C, et al. Adeno-associated virus vectored immunoprophylaxis to prevent HIV in healthy adults: a phase 1 randomised controlled trial. The Lancet HIV 2019; 6:e230-e9
11. Jayaraman M, Ansell S M, Mui B L, Tam Y K, Chen J, Du X, et al. Maximizing the potency of siRNA lipid nanoparticles for hepatic gene silencing in vivo. Angewandte Chemie (International ed in English) 2012; 51:8529-33
12. Miao L, Li L, Huang Y, Delcassian D, Chahal J, Han J, et al. Delivery of mRNA vaccines with heterocyclic lipids increases anti-tumor efficacy by STING-mediated immune cell activation. Nature Biotechnology 2019; 37:1174-85
13. Kauffman K J, Dorkin J R, Yang J H, Heartlein M W, DeRosa F, Mir F F, et al. Optimization of Lipid Nanoparticle Formulations for mRNA Delivery in Vivo with Fractional Factorial and Definitive Screening Designs. Nano Letters 2015; 15:7300-6
14. Semple S C, Akinc A, Chen J, Sandhu A P, Mui B L, Cho C K, et al. Rational design of cationic lipids for siRNA delivery. Nature Biotechnology 2010; 28:172-6
15. Gu F, Zhang L, Teply B A, Mann N, Wang A, Radovic-Moreno A F, et al. Precise engineering of targeted nanoparticles by using self-assembled biointegrated block copolymers. Proceedings of the National Academy of Sciences 2008; 105:2586 LP-91
16. Akinc A, Zumbuehl A, Goldberg M, Leshchiner E S, Busini V, Hossain N, et al. A combinatorial library of lipid-like materials for delivery of RNAi therapeutics. Nature biotechnology 2008; 26:561-9
17. Sabnis S, Kumarasinghe E S, Salerno T, Mihai C, Ketova T, Senn J J, et al. A Novel Amino Lipid Series for mRNA Delivery: Improved Endosomal Escape and Sustained Pharmacology and Safety in Non-human Primates. Molecular therapy: the journal of the American Society of Gene Therapy 2018; 26:1509-19
18. Akinc A, Maier M A, Manoharan M, Fitzgerald K, Jayaraman M, Barros S, et al. The Onpattro story and the clinical translation of nanomedicines containing nucleic acid-based drugs. Nature Nanotechnology 2019; 14:1084-7
19. Wan C, Allen T M, Cullis P R. Lipid nanoparticle delivery systems for siRNA-based therapeutics. Drug Delivery and Translational Research 2014; 4:74-83
20. Cullis P R, Hope M J. Lipid Nanoparticle Systems for Enabling Gene Therapies. Molecular Therapy 2017; 25:1467-75
21. Reichmuth A M, Oberli M A, Jaklenec A, Langer R, Blankschtein D. mRNA vaccine delivery using lipid nanoparticles. Ther Deliv 2016; 7:319-34
22. Kulkarni J A, Cullis P R, Van Der Meel R. Lipid nanoparticles enabling gene therapies: from concepts to clinical utility. Nucleic acid therapeutics 2018; 28:146-57
23. Hassett K J, Benenato K E, Jacquinet E, Lee A, Woods A, Yuzhakov O, et al. Optimization of Lipid Nanoparticles for Intramuscular Administration of mRNA Vaccines. Molecular Therapy—Nucleic Acids 2019; 15:1-11
24. Xue H Y, Liu S, Wong H L. Nanotoxicity: a key obstacle to clinical translation of siRNA-based nanomedicine. Nanomedicine (London, England) 2014; 9:295-312
25. Sedic M, Senn J J, Lynn A, Laska M, Smith M, Platz S J, et al. Safety Evaluation of Lipid Nanoparticle-Formulated Modified mRNA in the Sprague-Dawley Rat and Cynomolgus Monkey. Veterinary Pathology 2017; 55:341-54
26. Zhang J-S, Liu F, Huang L. Implications of pharmacokinetic behavior of lipoplex for its inflammatory toxicity. Advanced Drug Delivery Reviews 2005; 57:689-98
27. Zolnik B S, Gonzdlez-Ferndndez A, Sadrieh N, Dobrovolskaia M A. Nanoparticles and the immune system. Endocrinology 2010; 151:458-65
28. Kedmi R, Ben-Arie N, Peer D. The systemic toxicity of positively charged lipid nanoparticles and the role of Toll-like receptor 4 in immune activation. Biomaterials 2010; 31:6867-75
29. Yanasarn N, Sloat B R, Cui Z. Negatively charged liposomes show potent adjuvant activity when simply admixed with protein antigens. Mol Pharm 2011; 8:1174-85
30. Peeples L. News feature: Avoiding pitfalls in the pursuit of a COVID-19. Vaccin Proc Natl Acad Sci 2020; 117
31. Inglut C T, Sorrin A J, Kuruppu T, Vig S, Cicalo J, Ahmad H, et al. Immunological and Toxicological Considerations for the Design of Liposomes. Nanomaterials (Basel) 2020; 10
32. Goncalves GAR, Paiva RdMA. Gene therapy: advances, challenges and perspectives. Einstein (Sao Paulo, Brazil) 2017; 15:369-75
33. Szebeni J. Complement activation-related pseudoallergy: A stress reaction in blood triggered by nanomedicines and biologicals. Molecular Immunology 2014; 61:163-73
34. Szebeni J, Bed6cs P, Rozsnyay Z, Weiszhár Z, Urbanics R, Rosivall L, et al. Liposome-induced complement activation and related cardiopulmonary distress in pigs: factors promoting reactogenicity of Doxil and AmBisome. Nanomedicine: Nanotechnology, Biology and Medicine 2012; 8:176-84
35. White J M, Delos S E, Brecher M, Schornberg K. Structures and mechanisms of viral membrane fusion proteins: multiple variations on a common theme. Crit Rev Biochem Mol Biol 2008; 43:189-219
36. Ciechonska M, Duncan R. Reovirus FAST proteins: virus-encoded cellular fusogens. Trends in microbiology 2014; 22:715-24
37. Duncan R. Fusogenic Reoviruses and Their Fusion-Associated Small Transmembrane (FAST) Proteins. Annual review of virology 2019; 6:341-63
38. Top D, de Antueno R, Salsman J, Corcoran J, Mader J, Hoskin D, et al. Liposome reconstitution of a minimal protein-mediated membrane fusion machine. The EMBO journal 2005; 24:2980-8
39. Clancy E K, Duncan R. Helix-Destabilizing, β-Branched, and Polar Residues in the Baboon Reovirus p15 Transmembrane Domain Influence the Modularity of FAST Proteins. Journal of Virology 2011; 85:4707 LP-19
40. Clancy E K, Duncan R. Reovirus FAST protein transmembrane domains function in a modular, primary sequence-independent manner to mediate cell-cell membrane fusion. J Virol 2009; 83:2941-50
41. Yin H, Kanasty R L, Eltoukhy A A, Vegas A J, Dorkin J R, Anderson D G. Non-viral vectors for gene-based therapy. Nature Reviews Genetics 2014; 15:541-55
42. Waehler R, Russell S J, Curiel D T. Engineering targeted viral vectors for gene therapy. Nature reviews Genetics 2007; 8:573-87
43. Baum C, Kustikova O, Modlich U, Li Z, Fehse B. Mutagenesis and Oncogenesis by Chromosomal Insertion of Gene Transfer Vectors. Human Gene Therapy 2006; 17:253-63
44. Bahl K, Senn J J, Yuzhakov O, Bulychev A, Brito L A, Hassett K J, et al. Preclinical and Clinical Demonstration of Immunogenicity by mRNA Vaccines against H10N8 and H7N9 Influenza Viruses. Molecular therapy: the journal of the American Society of Gene Therapy 2017; 25:1316-27
45. Ickenstein L M, Garidel P. Lipid-based nanoparticle formulations for small molecules and RNA drugs. Expert Opinion on Drug Delivery 2019; 16:1205-26
46. Dawe S, Corcoran J A, Clancy E K, Salsman J, Duncan R. Unusual topological arrangement of structural motifs in the baboon reovirus fusion-associated small transmembrane protein. J Virol 2005; 79:6216-26
47. Salsman J, Top D, Boutilier J, Duncan R. Extensive syncytium formation mediated by the reovirus FAST proteins triggers apoptosis-induced membrane instability. J Virol 2005; 79:8090-100
48. Corcoran J A, Duncan R. Reptilian Reovirus Utilizes a Small Type Ill Protein with an External Myristylated Amino Terminus To Mediate Cell-Cell Fusion. Journal of Virology 2004; 78:4342
49. Top D, Read J A, Dawe S J, Syvitski R T, Duncan R. Cell-cell membrane fusion induced by p15 fusion-associated small transmembrane (FAST) protein requires a novel fusion peptide motif containing a myristoylated polyproline type II helix. J Biol Chem 2012; 287:3403-14
50. Parmar H B, Barry C, Duncan R. Polybasic trafficking signal mediates golgi export, ER retention or ER export and retrieval based on membrane-proximity. PLoS One 2014; 9:e94194
51. Read J, Clancy E K, Sarker M, de Antueno R, Langelaan D N, Parmar H B, et al. Reovirus FAST Proteins Drive Pore Formation and Syncytiogenesis Using a Novel Helix-Loop-Helix Fusion-Inducing Lipid Packing Sensor. PLoS Pathog 2015; 11:e1004962
52. Clancy E K, Duncan R. Helix-destabilizing, beta-branched, and polar residues in the baboon reovirus p15 transmembrane domain influence the modularity of FAST proteins. J Virol 2011; 85:4707-19
53. Belliveau N M, Huft J, Lin P J, Chen S, Leung A K, Leaver T J, et al. Microfluidic Synthesis of Highly Potent Limit-size Lipid Nanoparticles for In Vivo Delivery of siRNA. Molecular therapy Nucleic acids 2012; 1:e37-e
54. Leung A K K, Tam Y Y C, Chen S, Hafez I M, Cullis P R. Microfluidic Mixing: A General Method for Encapsulating Macromolecules in Lipid Nanoparticle Systems. The Journal of Physical Chemistry B 2015; 119:8698-706
55. Chen D, Love K T, Chen Y, Eltoukhy A A, Kastrup C, Sahay G, et al. Rapid Discovery of Potent siRNA-Containing Lipid Nanoparticles Enabled by Controlled Microfluidic Formulation. Journal of the American Chemical Society 2012; 134:6948-51
56. Elsabahy M, Wooley K L. Cytokines as biomarkers of nanoparticle immunotoxicity. Chemical Society reviews 2013; 42:5552-76
57. Lv H, Zhang S, Wang B, Cui S, Yan J. Toxicity of cationic lipids and cationic polymers in gene delivery. Journal of Controlled Release 2006; 114:100-9
58. Goswami R, Subramanian G, Silayeva L, Newkirk I, Doctor D, Chawla K, et al. Gene Therapy Leaves a Vicious Cycle. Frontiers in oncology 2019; 9:297-
59. Arteta M Y, Kjellman T, Bartesaghi S, Wallin S, Wu X, Kvist A J, et al. Successful reprogramming of cellular protein production through mRNA delivered by functionalized lipid nanoparticles. Proceedings of the National Academy of Sciences 2018; 115:E3351-E60
60. Lowenstine L J. A primer of primate pathology: lesions and nonlesions. Toxicologic Pathology 2003; 31:92-102
61. Ward K W, Coon D J, Magiera D, Bhadresa S, Nisbett E, Lawrence M S. Exploration of the African green monkey as a preclinical pharmacokinetic model: intravenous pharmacokinetic parameters. Drug Metab Dispos 2008; 36:715-20
62. Lee S J, McPherron A C. Regulation of myostatin activity and muscle growth. Proceedings of the National Academy of Sciences of the United States of America 2001; 98:9306-11
63. Mendell J R, A I-Zaidy S, Shell R, Arnold W D, Rodino-Klapac L R, Prior T W, et al. Single-Dose Gene-Replacement Therapy for Spinal Muscular Atrophy. New England Journal of Medicine 2017; 377:1713-22
64. A I-Zaidy S A, Sahenk Z, Rodino-Klapac L R, Kaspar B, Mendell J R. Follistatin Gene Therapy Improves Ambulation in Becker Muscular Dystrophy. Journal of neuromuscular diseases 2015; 2:185-92
65. Winbanks C E, Weeks K L, Thomson R E, Sepulveda P V, Beyer C, Qian H, et al. Follistatin-mediated skeletal muscle hypertrophy is regulated by Smad3 and mTOR independently of myostatin. The Journal of cell biology 2012; 197:997-1008
66. Hansen J S, Rutti S, Arous C, Clemmesen J O, Secher N H, Drescher A, et al. Circulating Follistatin Is Liver-Derived and Regulated by the Glucagon-to-Insulin Ratio. The Journal of Clinical Endocrinology & Metabolism 2016; 101:550-60
67. Cho C F, Yu L, Nsiama T K, Kadam A N, Raturi A, Shukla S, et al. Viral nanoparticles decorated with novel EGFL7 ligands enable intravital imaging of tumor neovasculature. Nanoscale 2017; 9:12096-109
68. Wang B, Wu H, Chai C, Lewis J, Pichiorri F, Eisenstat D D, et al. MicroRNA-1301 suppresses tumor cell migration and invasion by targeting the p53/UBE4B pathway in multiple human cancer cells. Cancer Letters 2017; 401:20-32
69. Cho C F, Shukla S, Simpson E J, Steinmetz N F, Luyt L G, Lewis J D. Molecular targeted viral nanoparticles as tools for imaging cancer. Methods Mol Biol 2014; 1108:211-30
70. Wang H, Yang Z, Gu J. Therapeutic Targeting of Angiogenesis with a Recombinant CTT Peptideâ “Endostatin Mimicâ” Kringle 5 Protein. 2014
71. Perez-Medina C, Abdel-Atti D, Zhang Y, Longo V A, Irwin C P, Binderup T, et al. A modular labeling strategy for in vivo PET and near-infrared fluorescence imaging of nanoparticle tumor targeting. J Nucl Med 2014; 55:1706-11
72. Lu C, McFarland M S, Nesbitt R L, Williams A K, Chan S, Gomez-Lemus J, et al. Ghrelin receptor as a novel imaging target for prostatic neoplasms. Prostate 2012; 72:825-33
73. Cho C-F, Amadei G A, Breadner D, Luyt L G, Lewis J D. Discovery of Novel Integrin Ligands from Combinatorial Libraries Using a Multiplex “Beads on a Bead” Approach. Nano Letters 2012; 12:5957-65
74. Pink D B S, Schulte W, Parseghian M H, Zijlstra A, Lewis J D. Real-time visualization and quantitation of vascular permeability in vivo: implications for drug delivery. PloS one 2012; 7:e33760-e
75. Mena E, Lindenberg L M, Choyke P L. New targets for PET molecular imaging of prostate cancer. 2019. Elsevier.
76. Mriouah J, Nesbitt R L, Richter S, Wuest M, Pink D, Sosnowski D, et al. Abstract 5143: Fusogenic targeted liposomes: novel nanotherapy for specific treatment of prostate cancer. Cancer Research 2017; 77:5143-
77. Steinmetz N F, Ablack A L, Hickey J L, Ablack J, Manocha B, Mymryk J S, et al. Intravital imaging of human prostate cancer using viral nanoparticles targeted to gastrin-releasing Peptide receptors. Small 2011; 7:1664-72
78. Martin A L, Hickey J L, Ablack A L, Lewis J D, Luyt L G, Gillies E R. Synthesis of bombesin-functionalized iron oxide nanoparticles and their specific uptake in prostate cancer cells. Journal of nanoparticle research: an interdisciplinary forum for nanoscale science and technology 2009; 12:1599-608
79. Ischia J, Patel O, Bolton D, Shulkes A, Baldwin G S. Expression and function of gastrin-releasing peptide (GRP) in normal and cancerous urological tissues. BJU international 2014; 113:40-7
80. Ross J S, Slodkowska E A, Symmans W F, Pusztai L, Ravdin P M, Hortobagyi G N. The HER-2 receptor and breast cancer: ten years of targeted anti-HER-2 therapy and personalized medicine. Oncologist 2009; 14:320-68
81. Heidenreich A, Bastian P J, Bellmunt J, Bolla M, Joniau S, van der Kwast T, et al. EAU guidelines on prostate cancer. Part II: Treatment of advanced, relapsing, and castration-resistant prostate cancer. Eur Urol 2014; 65:467-79
82. Cutumisu M, Vasquez C, Uhlich M, Beatty P H, Hamayeli-Mehrabani H, Djebah R, et al. PROSPeCT: A Predictive Research Online System for Prostate Cancer Tasks. JCO-CCI 2019:1-12
83. Dinakaran D, Chen H, Wuest M, Wuest F, Murray D, Usmani N, et al. 6 Novel X-RAY Activated Photodynamic (RadioPDT) Nanoparticles for Deep Seated Tumours. Radiotherapy and Oncology 2019; 139:S6
84. Evans S M, Millar J L, Moore C M, Lewis J D, Huland H, Sampurno F, et al. Cohort profile: the TrueNTH Global Registry—an international registry to monitor and improve localised prostate cancer health outcomes. BMJ Open 2017; 7:e017006
85. Wang T, Abou-Ouf H, Hegazy S A, Alshalalfa M, Stoletov K, Lewis J, et al. Ankyrin G expression is associated with androgen receptor stability, invasiveness, and lethal outcome in prostate cancer patients. Journal of Molecular Medicine 2016; 94:1411-22
86. Puri A, Kramer-Marek G, Campbell-Massa R, Yavlovich A, Tele S C, Lee S B, et al. HER2-specific affibody-conjugated thermosensitive liposomes (Affisomes) for improved delivery of anticancer agents. J Liposome Res 2008; 18:293-307
87. Barry C, Duncan R. Multifaceted sequence-dependent and -independent roles for reovirus FAST protein cytoplasmic tails in fusion pore formation and syncytiogenesis. J Virol 2009; 83:12185-95
88. Uversky V N. Intrinsically disordered proteins from A to Z. The international journal of biochemistry & cell biology 2011; 43:1090-103
89. Parmar H B, Barry C, Kai F, Duncan R. Golgi complex-plasma membrane trafficking directed by an autonomous, tribasic Golgi export signal. Molecular Biology of the Cell 2014; 25:866-78
90. Read J, Clancy E K, Sarker M, de Antueno R, Langelaan D N, Parmar H B, et al. Reovirus FAST Proteins Drive Pore Formation and Syncytiogenesis Using a Novel Helix-Loop-Helix Fusion-Inducing Lipid Packing Sensor. PLOS Pathogens 2015; 11:e1004962
91. Corcoran J A, Syvitski R, Top D, Epand R M, Epand R F, Jakeman D, et al. Myristoylation, a protruding loop, and structural plasticity are essential features of a nonenveloped virus fusion peptide motif. J Biol Chem 2004; 279:51386-94
92. Fitzgerald K, White S, Borodovsky A, Bettencourt B R, Strahs A, Clausen V, et al. A Highly Durable RNAi Therapeutic Inhibitor of PCSK9. N Engl J Med 2017; 376:41-51
93. Fitzgerald K, White S, Borodovsky A, Bettencourt B R, Strahs A, Clausen V, et al. A Highly Durable RNAi Therapeutic Inhibitor of PCSK9. New England Journal of Medicine 2016; 376:41-51
94. Nathwani A C, Reiss U M, Tuddenham E G D, Rosales C, Chowdary P, McIntosh J, et al. Long-Term Safety and Efficacy of Factor IX Gene Therapy in Hemophilia B. New England Journal of Medicine 2014; 371:1994-2004
95. Keeler A M, Flotte T R. Recombinant adeno-associated virus gene therapy in light of Luxturna (and Zolgensma and Glybera): where are we, and how did we get here? Annual review of virology 2019; 6:601-21
96. Flotte T R, Schwiebert E M, Zeitlin P L, Carter B J, Guggino W B. Correlation between DNA transfer and cystic fibrosis airway epithelial cell correction after recombinant adeno-associated virus serotype 2 gene therapy. Human gene therapy 2005; 16:921-8
97. Flotte T R, Zeitlin P L, Reynolds T C, Heald A E, Pedersen P, Beck S, et al. Phase I Trial of Intranasal and Endobronchial Administration of a Recombinant Adeno-Associated Virus Serotype 2 (rAAV2)-CFTR Vector in Adult Cystic Fibrosis Patients: A Two-Part Clinical Study. Human Gene Therapy 2003; 14:1079-88
98. Manno C S, Pierce G F, Arruda V R, Glader B, Ragni M, Rasko J J E, et al. Successful transduction of liver in hemophilia by AAV-Factor IX and limitations imposed by the host immune response. Nature Medicine 2006; 12:342-7
99. Mingozzi F, Meulenberg J J, Hui D J, Basner-Tschakarjan E, Hasbrouck N C, Edmonson S A, et al. AAV-1-mediated gene transfer to skeletal muscle in humans results in dose-dependent activation of capsid-specific T cells. Blood 2009; 114:2077-86
100. Ambegia E, Ansell S, Cullis P, Heyes J, Palmer L, MacLachlan I. Stabilized plasmid-lipid particles containing PEG-diacylglycerols exhibit extended circulation lifetimes and tumor selective gene expression. Biochimica et Biophysica Acta (BBA)-Biomembranes 2005; 1669:155-63
101. Ambegia E, Ansell S, Cullis P, Heyes J, Palmer L, MacLachlan I. Stabilized plasmid-lipid particles containing PEG-diacylglycerols exhibit extended circulation lifetimes and tumor selective gene expression. Biochimica et Biophysica Acta (BBA)—Biomembranes 2005; 1669:155-63
102. Paunovska K, Gil C J, Lokugamage M P, Sago C D, Sato M, Lando G N, et al. Analyzing 2000 in vivo drug delivery data points reveals cholesterol structure impacts nanoparticle delivery. ACS nano 2018; 12:8341-9
103. Mendell J R, Sahenk Z, A I-Zaidy S, Rodino-Klapac L R, Lowes L P, Alfano L N, et al. Follistatin Gene Therapy for Sporadic Inclusion Body Myositis Improves Functional Outcomes. Molecular therapy: the journal of the American Society of Gene Therapy 2017; 25:870-9
104. Kota J, Handy C R, Haidet A M, Montgomery C L, Eagle A, Rodino-Klapac L R, et al. Follistatin gene delivery enhances muscle growth and strength in nonhuman primates. Science translational medicine 2009; 1:6ra15-6ra
105. Haidet A M, Rizo L, Handy C, Umapathi P, Eagle A, Shilling C, et al. Long-term enhancement of skeletal muscle mass and strength by single gene administration of myostatin inhibitors. Proceedings of the National Academy of Sciences of the United States of America 2008; 105:4318-22
106. Mendell J R, Sahenk Z, Malik V, Gomez A M, Flanigan K M, Lowes L P, et al. A phase 1/2a follistatin gene therapy trial for becker muscular dystrophy. Molecular therapy: the journal of the American Society of Gene Therapy 2015; 23:192-201
107. Raper S E, Chirmule N, Lee F S, Wivel N A, Bagg A, Gao G-p, et al. Fatal systemic inflammatory response syndrome in a ornithine transcarbamylase deficient patient following adenoviral gene transfer. Molecular Genetics and Metabolism 2003; 80:148-58
108. Wang D, Tai P W L, Gao G. Adeno-associated virus vector as a platform for gene therapy delivery. Nature reviews Drug discovery 2019; 18:358-78
109. Chew W L, Tabebordbar M, Cheng J K W, Mali P, Wu E Y, Ng A H M, et al. A multifunctional AAV-CRISPR-Cas9 and its host response. Nature Methods 2016; 13:868-74
110. Mali P, Yang L, Esvelt K M, Aach J, Guell M, DiCarlo J E, et al. RNA-Guided Human Genome Engineering via Cas9. Science 2013; 339:823-6
111. Sander J D, Joung J K. CRISPR-Cas systems for editing, regulating and targeting genomes. Nature Biotechnology 2014; 32:347-55
112. Colella P, Ronzitti G, Mingozzi F. Emerging issues in AAV-mediated in vivo gene therapy. Molecular Therapy-Methods & Clinical Development 2018; 8:87-104
113. Long C, Amoasii L, Mireault A A, McAnally J R, Li H, Sanchez-Ortiz E, et al. Postnatal genome editing partially restores dystrophin expression in a mouse model of muscular dystrophy. Science 2016; 351:400-3
114. Yin H, Song C-Q, Dorkin J R, Zhu L J, Li Y, Wu Q, et al. Therapeutic genome editing by combined viral and non-viral delivery of CRISPR system components in vivo. Nature Biotechnology 2016; 34:328-33
115. Baden L R, El Sahly H M, Essink B, Kotloff K, Frey S, Novak R, et al. Efficacy and safety of the mRNA-1273 SARS-CoV-2 vaccine. New England Journal of Medicine 2021; 384:403-16
116. Wu K, Choi A, Koch M, Ma L, Hill A, Nunna N, et al. Preliminary Analysis of Safety and Immunogenicity of a SARS-CoV-2 Variant Vaccine Booster. medRxiv 2021:2021.05.05.21256716
117. Sahin U, Muik A, Derhovanessian E, Vogler I, Kranz L M, Vormehr M, et al. COVID-19 vaccine BNT162b1 elicits human antibody and TH1 T cell responses. Nature 2020; 586:594-9
118. Corbett K S, Edwards D K, Leist S R, Abiona O M, Boyoglu-Barnum S, Gillespie R A, et al. SARS-CoV-2 mRNA vaccine design enabled by prototype pathogen preparedness. Nature 2020; 586:567-71
119. Anderson E J, Rouphael N G, Widge A T, Jackson L A, Roberts P C, Makhene M, et al. Safety and Immunogenicity of SARS-CoV-2 mRNA-1273 Vaccine in Older Adults. New England Journal of Medicine 2020
120. Jorritsma S H T, Gowans E J, Grubor-Bauk B, Wijesundara D K. Delivery methods to increase cellular uptake and immunogenicity of DNA vaccines. Vaccine 2016; 34:5488-94
121. Hannaman D, Dupuy L C, Ellefsen B, Schmaljohn C S. A Phase 1 clinical trial of a DNA vaccine for Venezuelan equine encephalitis delivered by intramuscular or intradermal electroporation. Vaccine 2016; 34:3607-12
122. Houser K V, Yamshchikov G V, Bellamy A R, May J, Enama M E, Sarwar U, et al. DNA vaccine priming for seasonal influenza vaccine in children and adolescents 6 to 17 years of age: A phase 1 randomized clinical trial. PloS one 2018; 13:e0206837-e
123. Modjarrad K, Roberts C C, Mills K T, Castellano A R, Paolino K, Muthumani K, et al. Safety and immunogenicity of an anti-Middle East respiratory syndrome coronavirus DNA vaccine: a phase 1, open-label, single-arm, dose-escalation trial. Lancet Infect Dis 2019; 19:1013-22
124. Tebas P, Kraynyak K A, Patel A, Maslow J N, Morrow M P, Sylvester A J, et al. Intradermal SynCon® Ebola G P DNA Vaccine Is Temperature Stable and Safely Demonstrates Cellular and Humoral Immunogenicity Advantages in Healthy Volunteers. The Journal of Infectious Diseases 2019; 220:400-10
125. Pardi N, Hogan M J, Porter F W, Weissman D. mRNA vaccines—a new era in vaccinology. Nat Rev Drug Discov 2018; 17
126. Basu A, Chakraborty S. Faculty Opinions recommendation of Zika virus protection by a single low-dose nucleoside-modified mRNA vaccination. Faculty Opinions—Post-Publication Peer Review of the Biomedical Literature: Faculty Opinions Ltd; 2017.
127. Jackson L A, Anderson E J, Rouphael N G, Roberts P C, Makhene M, Coler R N, et al. An mRNA Vaccine against SARS-CoV-2—Preliminary Report. New England Journal of Medicine 2020
128. Roche J A, Ford-Speelman D L, Ru L W, Densmore A L, Roche R, Reed P W, et al. Physiological and histological changes in skeletal muscle following in vivo gene transfer by electroporation. American journal of physiology Cell physiology 2011; 301:C1239-C50
129. Oba Y, Ojika M, Inouye S. Firefly luciferase is a bifunctional enzyme: ATP-dependent monooxygenase and a long chain fatty acyl-CoA synthetase. FEBS letters 2003; 540:251-4
130. Vizard D L, Wood D O, Papineni R V, Feke G D, Orton S P, McLaughlin W E. Analytical radiography for planar radiographic images implemented with a multi-modal system. Comput Methods Programs Biomed 2010; 99:88-97
131. Wen Y, Murach K A, Vechetti I J, Jr., Fry C S, Vickery C, Peterson C A, et al. MyoVision: software for automated high-content analysis of skeletal muscle immunohistochemistry. J Appl Physiol (1985) 2018; 124:40-51

1.-63. (canceled)

64. A method of making nucleic acid cargo-proteolipid vesicles, comprising:
providing an aqueous phase comprising a nucleic acid cargo and a recombinant fusion-associated small transmembrane (FAST) polypeptide;
providing an organic phase comprising an ionizable lipid, a helper lipid, and a PEGylated lipid; and
mixing the aqueous phase and the organic phase together, thereby forming a mixed phase comprising the nucleic acid cargo-proteolipid vesicles.

65. The method of claim 64 further comprising exchanging a solution of the mixed phase for a buffer.

66. The method of claim 65, further comprising concentrating the mixed phase during or after the exchanging of the solution of the mixed phase, to form a concentrated product enriched for the nucleic acid cargo-proteolipid vesicles.

67. The method of claim 66, further comprising filtering the concentrated product to form a filtered product comprising the nucleic acid cargo-proteolipid vesicles.

68. The method of claim 64, wherein mixing the aqueous phase and the organic phase together comprises combining the aqueous phase and the organic phase at ratio of about 3:1 of aqueous phase:organic phase.

69. The method of claim 64, further comprising:
combining the ionizable lipid, helper lipid, and PEGylated lipid together to form combined lipids;
dehydrating the combined lipids; and
rehydrating the combined lipids in an organic solvent, thereby generating the organic phase.

70. The method of claim 64, wherein the organic phase further comprises ethanol.

71. The method of claim 64, wherein the ionizable lipid is Dlin-MC3-DMA, 1,2-dioleyloxy-3-dimethylaminopropane (DODMA), or 1,2-dioleoyl-3-dimethylammonium-propane (DODAP), or a mixture thereof.

72. The method of claim 64, wherein the ionizable lipid is DODAP.

73. The method of claim 64, wherein the helper lipid is 2-dioleoyl-sn-glycero-3-phosphoethanolamine (DOPE).

74. The method of claim 64, wherein the PEGylated lipid is 1,2-dimyristoyl-sn-glycero-3-methoxypolyethylene glycol-2000 (DMG-PEG).

75. The method of claim 64, wherein the organic phase comprises the ionizable lipid, helper lipid, and PEGylated lipid at a molar ratio of about 66:30:4 of ionizable lipid:helper lipid:PEGylated lipid.

76. The method of claim 75, wherein the ionizable lipid is DODAP, the helper lipid is DOPE, and the PEGylated lipid is DMG-PEG.

77. The method of claim 64, wherein organic phase further comprises a cationic lipid.

78. The method of claim 77, wherein the organic phase comprises the cationic lipid, the ionizable lipid, the helper lipid, and the PEGylated lipid at a molar ratio of about 6:60:30:4 of cationic lipid:ionizable lipid:helper lipid:PEGylated lipid.

79. The method of claim 64, wherein the cationic lipid is 1,2-di-O-octadecenyl-3-trimethylammonium propane (DOTMA) or 1,2-dioleoyl-3-trimethylammonium-propane (DOTAP), or a mixture thereof.

80. The method of claim 77, wherein the cationic lipid is DOTAP, the ionizable lipid is DODAP, the helper lipid is DOPE, and the PEGylated lipid is DMG-PEG.

81. The method of claim 80, wherein the organic phase comprises the DOTAP, DODAP, DOPE and DMG-PEG at a molar ratio of about 6:60:30:4 of DOTAP:DODAP:DOPE:DMG-PEG.

82. The method of claim 64, wherein the FAST polypeptide comprises a p10 FAST protein, a p13 FAST protein, a p14 FAST protein, a p15 FAST protein, a p16 FAST protein, or a p22 FAST protein, or a fragment thereof.

83. The method of claim 64, wherein the FAST polypeptide comprises a fusion protein comprising a p14 ectodomain and a p15 endodomain.

84. The method of claim 64, wherein the FAST polypeptide comprises a fusion protein comprising a p14 ectodomain comprising an amino acid sequence at least 90% identical to SEQ ID NO: 2, a p14 or p15 transmembrane domain, and a p15 endodomain comprising an amino acid sequence at least 90% identical to SEQ ID NO: 3 or 4.

85. The method of claim 64, wherein the nucleic acid cargo comprises DNA.
